(19) United States
(12) Patent Application Publication
(10) Pub. No.: US 2004/0005537 A1
(43) Pub. Date: Jan. 8, 2004

(54) Method of identifying toxic agents using differential gene expression

(75) Inventors: Bonnie Gould-Rothberg, Guilford, CT (US); Vincent Dipippo, East Haven, CT (US); Kellye K. Daniels, Kirkland, WA (US)

Correspondence Address: MINTZ, LEVIN, COHN, FERRIS, GLOVSKY, AND POPEO, P.C., ONE FINANCIAL CENTER, BOSTON, MA, 02111, US

(73) Assignee: CuraGen Corporation, New Haven, CT

(21) Appl. No.: 10/428,681

(22) Filed: May 2, 2003

Related U.S. Application Data
(63) Continuation of application No. 09/791,945, filed on Feb. 22, 2001, now abandoned.
(60) Provisional application No. 60/184,017, filed on Feb. 22, 2000.
(60) Provisional application No. 60/213,027, filed on Jun. 21, 2000.
(60) Provisional application No. 60/239,535, filed on Oct. 10, 2000.

(51) Int. Cl. C12Q 1/00; C12Q 1/68

(52) U.S. Cl. 435/4; 435/6

(57) ABSTRACT

Disclosed are methods of identifying toxic agents, e.g., cardiotoxic agents, using differential gene expression. Also disclosed are novel nucleic acid sequences whose expression is differentially regulated by serotonin modulating agents.

### RELATED APPLICATIONS

This application claims priority to U.S. S No. 60/184,017, filed Feb. 22, 2000 U.S. S No. 60/213,027, filed Jun. 21, 2000 and U.S. S No. 60/239,535, filed Oct. 10, 2000 which are incorporated herein by reference in their entirety

### FIELD OF THE INVENTION

The invention relates generally to the identification of cardiotoxic agents in heart tissue using differential gene expression.

### BACKGROUND OF THE INVENTION

An unfortunate drawback associated with otherwise promising drugs is that they induce unwanted side effects as well as their intended therapeutic effects. Often, these side effects do not become apparent until the drug has entered, or even completed, clinical trials. For example, the serotonin reuptake inhibitors, dexfenfluramine (Redux) and fenfluramine (Pondimin), have been recently used to treat obesity. In spite of their demonstrated effectiveness as anorectic agents, significant side affects have been associated with these compounds. In particular, it has been reported to result in valvular heart disease in a subset of patients to which they are administered.

Cardiotoxicity associated with administration of dexfenfluramine and fenfluramine can range from pulmonary hypertension, valvular heart disease and death. Clinical manifestation can include shortness of breath, fatigue, swelling of the feet, chest pain and heart murmur. Histopathologic findings included plaque-like encasement of the leaflets and chordal structures with a “stuck-on” appearance and intact valve architecture. In addition, valve features are identical to those seen in ergotamine toxicity or carcinoid disease.

### SUMMARY OF THE INVENTION

The invention is based in part on the discovery that certain nucleic acids are differentially expressed in cardiac tissue of animals treated with cardiotoxic serotonin modulators (e g, dexfenfluramine fenfluramine and dihydroergotamine) compared with non-cardiotoxic serotonin modulators (e.g., fluoxetine, sibutamine, and sumatriptan). These differentially expressed nucleic acids include novel sequences and nucleic acids sequences that, while previously described, have not heretofore been identified as serotonin modulator responsive.

In various aspects, the invention includes methods of method of screening a test agent for toxicity, e.g., cardiotoxicity. For example, in one aspect, the invention provides a method of identifying a cardiotoxic agent by providing a test cell population comprising a cell capable of expressing one or more nucleic acids sequences responsive to serotonin modulators, contacting the test cell population with the test agent and comparing the expression of the nucleic acids sequences in the test cell population to the expression of the nucleic acids sequences in a reference cell population not treated with a serotonin modulator An alteration in expression of the nucleic acids sequences in the test cell population compared to the expression of the gene in the reference cell population indicates that the test agent is cardiotoxic.

In an another aspect, the invention provides a method of assessing the cardiotoxicity of a test agent in a subject. The method includes providing from the subject a cell population comprising a cell capable of expressing one or more dexfenfluramine and fenfluramine responsive genes, and comparing the expression of the nucleic acids sequences to the expression of the nucleic acids sequences in a reference cell population that includes cells from a subject whose exposure status to a cardiotoxic agent is known. An alteration in expression of the in the test cell population compared to the expression of the nucleic acids sequences in the reference cell population indicates the cardiotoxicity of the test agent in the subject.

In further aspect, the invention provides a method of screening a test agent serotonin modulating activity. For example, in one aspect, the invention provides a method of identifying a serotonin modulating agent by providing a test cell population comprising a cell capable of expressing one or more nucleic acids sequences responsive to serotonin modulators, contacting the test cell population with the test agent and comparing the expression of the nucleic acids sequences in the test cell population to the expression of the nucleic acids sequences in a reference cell population not treated with a serotonin modulators. An alteration in expression of the nucleic acids sequences in the test cell population compared to the expression of the gene in the reference cell population indicates that the test agent is a serotonin modulator.

Also provided are novel nucleic acids, as well as their encoded polypeptides, whose expression is responsive to the effects of serotonin modulators.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below All publications, patent applications, patents, and other references mentioned herein are incorporated by reference in their entirety In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention is based in part on the discovery of changes in expression patterns of multiple nucleic acid sequences in rodent heart cells following exposure to serotonin modulating agents. The serotin modulating agents included the serotoin uptake inhibitors, dexfenfluramine, fenfluramine, fluxetine, sibutamine; the selective serotonin receptor agonist, sumatriptan; and the non-selective serotonergic agonist dihydroergotamine.

The differentially expressed nucleic acids were identified by administering the LD₁₀ dose of each serotin modulating agent to 12 week old male Sprague Dawley rats for three days. Control animals received sterile water or canola oil. The animals were sacrificed 24 hours following the last dose. Liver tissue was dissected from the animals, and total RNA was recovered from the dissected tissue. cDNA was prepared and the resulting samples were processed through using GENECALLING™ differential expression analysis as described in U.S. Pat. No. 5,871,697 and in Shimkets et al., Nature Biotechnology 17:798-803 (1999). The contents of these patents and publications are incorporated herein by reference in their entirety.

Thousands of gene fragments were initially found to be differentially expressed in rat heart tissue in response to serotonin modulating agents in. Genes fragments whose expression levels were modulated greater than ±1.5-fold were selected for further analysis.

A summary of the sequences analyzed are presented in Table 1. Column 6 of Table 1, entitled “Function”, lists the type of classification assigned for the protein, based on its function. The 210 single nucleic acid sequences identified herein, are referred to herein as CARDIOTOX 1-210.

Differential expression of CARDIOTOX 1-139 gene fragments was confirmed using a unlabeled oligonucleotide competition assay as described in Shimkets et al., Nature Biotechnology 17:198-803. The mitocondrial gene fragments (CARDIOTOX 140-210) were not subjected to further analysis due to the suprisingly large number of fragments identified. However all the serotonin modulating agent had a significant impact on the of mitochondrial genes critical to the oxidative phosphorylation pathway. This finding is significant as an impaired oxidative phosphorylation pathway will increase the amount of reactive oxygen species within an organ and, in turn, increase the potential for cardiac damage. Thus, these genes are potential useful general toxicity markers for the serotonin modulators.

Seventy-three sequences (CARDIOTX: 1-7, 10-13, 19-34, 45-53, 58-85, 111-113, 120, 130, 132-134 and 138) represent novel rat genes for which the sequence identity to sequences found in public databases suggesting a putative homology.

The 137 other sequenced identified have been previously described. For some of the novel sequences (i.e., CARDIOTX: 1-7, 10-13, 19-34, 45-53, 58-85, 111-113, 120, 130, 132-134 and 138), a cloned sequence is provided along with one or more additional sequence fragments (e.g., ESTs or contigs) which contain sequences substantially identical to, the cloned sequence. Also provided is a consensus sequences which includes a composite sequence assembled from the cloned and additional fragments. For a given CARDIOTOX sequence, its expression can be measured using any of the associated nucleic acid sequences may be used in the methods described herein. For previously described sequences database accession numbers are provided. This information allows for one of ordinary skill in the art to deduce information necessary for detecting and measuring expression of the CARDIOTOX nucleic acid sequences.

By comparing of the genes differentially expressed in response to the various serototin modulating agents it was possible to generate gene profiles capable of distinguishing between cardiotoxic (dexfenfluramine, fenfluramine and dihydroerogtamine) and noncardiotoxic (fluoxetine, sibutramine and sumatriptan) serotonin modulationg agents.

The serotonin modulating agent responsive nucleic acids discussed herein include the following:

TABLE 1

| | | Effects on Transcription Level | | | | | | | | | |
|---|---|---|---|---|---|---|---|---|---|---|---|
| | | | | | | Di- | | | | | |
| | | Dexfen- | Fen- | Flu- | Sibu- | hydro- | Suma- | | | | |
| | | flura- | flura- | oxe- | tra- | ergot- | trip- | | | | |
| | GenBank | mine | mine | tine | mine | amine | tan | | | CARDIOTOX | |
| Description of Sequence | Acc# | (12271) | (12272) | (12252) | (12246) | (12251) | (12253) | | Functional Role | Assignment | SEQ ID NO |
| DEXFENFLURAMINE MODULATED ONLY | | | | | | | | | | | |
| Novel gene fragment, 524 bp, 86% SI to mouse F-box | N/A | +1.5 | +1.4 | +1.3 | +1.3 | +1.3 | +1.5 | 01.05.02 | UBIQUITIN CYCLE | 1 | 1, 2 |
| protein FBX6b [AF176526] | | | | | | | | | | | |
| Novel gene fragment, 306 bp, 91% SI to mouse low | N/A | −1.8 | −1.1 | −1.3 | −1.3 | −1.4 | −1.6 | 04.01 | LIPID METABOLISM | 2 | 3 |
| density lipoprotein receptor related protein 4 [AB013874] | | | | | | | | | | | |
| Novel gene fragment, 540 bp, 97% SI to mouse skeletal | N/A | +2.3 | +1.3 | −1.2 | −1.1 | +1.2 | +2.4 | 05.01.01.03 | STRUCTURAL ARM: ACTINS & SHORT | 3 | 4, 5 |
| muscle alpha-actin [X03766] | | | | | | | | | FILAMENTS | | |
| Novel gene fragment, 80 bp | N/A | −1.6 | −1.1 | −1.3 | −1.3 | −1.4 | −1.4 | 09 | UNKNOWN FUNCTION | 4 | 6 |
| Novel gene fragment, 957 bp, 99% SI to kidney injury | N/A | −1.6 | −1.4 | −1.4 | −1.0 | −1.4 | −1.6 | 09 | UNKNOWN FUNCTION | 5 | 7, 8 |
| associated moleculer HW036 [V80591] (from patent | | | | | | | | | | | |
| database) | | | | | | | | | | | |
| Novel gene fragment, 282 bp, 85% SI to human | N/A | −1.7 | −1.3 | −1.2 | −1.4 | −1.2 | −1.7 | 09 | UNKNOWN FUNCTION | 6 | 9 |
| KIAA1515 protein [AB040948] | | | | | | | | | | | |
| Novel gene fragment, 405 bp, 88% SI to human 2- | N/A | +1.5 | +1.4 | +1.2 | −1.4 | +1.3 | +1.5 | | | 7 | 10, 11 |
| oxoglutarate dehydrogenase [D10523] | | | | | | | | | | | |
| UCP2 | AB010743 | −1.6 | −1.3 | −1.3 | −1.3 | +1.0 | −1.6 | 04.04.03 | ATP/PROTON MOTIVE FORCE | 8 | |
| | | | | | | | | | INTERCONVERSION | | |
| Plasma membrane Ca2 + ATPase-isoform 1 | J03753 | −1.6 | −1.3 | −1.1 | +1.2 | −1.4 | −1.6 | 04.11.02.02 | CATIONS | 9 | |
| FENFLURAMINE MODULATED ONLY | | | | | | | | | | | |
| Novel gene fragment, 242 bp, 97% SI to mouse HSP86 | N/A | +1.4 | +1.6 | +1.4 | +1.4 | +1.0 | +1.4 | 01.03.01 | MOLECULAR CHAPERONE | 10 | 12, 13 |
| heat-shock protein [X16857] | | | | | | | | | | | |
| Novel gene fragment, 280 bp, 65% SI to human TRF1- | N/A | +1.4 | +1.6 | +1.3 | +1.2 | +1.3 | +1.4 | 05 | TISSUE ARCHITECTURE | 11 | 14 |
| interacting, ankyrin-related ADP-ribose polymerase | | | | | | | | | | | |
| [AF082556] | | | | | | | | | | | |
| Novel gene fragment, 348 bp, 88% SI to mouse Sec61 | N/A | +1.3 | +1.6 | +1.2 | +1.4 | +1.4 | +1.3 | 07.02.02 | TRANSMEMBRANE PROTEINS | 12 | 15, 16 |
| protein complex gamma subunit [U11027] | | | | | | | | | | | |
| Novel gene fragment 553 bp, 93% SI to mouse Sid329 | N/A | +1.4 | +1.5 | +1.1 | +1.1 | +1.2 | +1.4 | 09 | UNKNOWN FUNCTION | 13 | 17, 18 |
| [AB024984] | | | | | | | | | | | |
| Kruppel-like transcription factor | AB020759 | +1.3 | +1.7 | +1.3 | +1.1 | +1.2 | +1.3 | 01.01 | mRNA TRANSCRIPTION | 14 | |
| Ribosomal protein L3 | X62166 | +1.4 | +2.3 | +1.1 | +1.3 | +1.4 | +1.4 | 01.02.01 | RIBOSOMAL PROTEIN | 15 | |
| Glucose-regulated protein (GRP) 75 | S78556 | +1.4 | +1.5 | +1.3 | +1.1 | +1.2 | +1.4 | 01.03.01 | MOLECULAR CHAPERONE | 16 | |
| Immunoglobulin heavy chain binding protein (BiP) | M14050 | +1.4 | +2.0 | +1.4 | −1.1 | +1.2 | +1.4 | 01.03.01 | MOLECULAR CHAPERONE | 17 | |
| Membrane-spannining proteoglycan NG2 | X56541 | −1.4 | −1.5 | −1.2 | −1.1 | −1.4 | −1.5 | 05.02 | EXTRACELLULAR MATRIX | 18 | |

DEXFENFLURAMINE AND FENFLURAMINE MODULATED ONLY

| | | | | | | | | | | | |
|---|---|---|---|---|---|---|---|---|---|---|---|
| Novel gene fragment, 1294 bp, 96% SI to mouse Sui 1 | N/A | +1.6 | +1.6 | +1.1 | +1.1 | +1.1 | +1.6 | 01.02.05 | TRANSLATION FACTORS | 19 | 19, 20, 21 |
| (elF) homolog [AF129888] | | | | | | | | | | | |
| Novel gene fragment, 723 bp, 85% SI to human | N/A | +1.6 | +1.5 | +1.0 | −1.1 | +1.2 | +1.6 | 01.02.05 | TRANSLATION FACTORS | 20 | 22, 23 |
| translation initiation factor elF3 p40 subunit [U54559] | | | | | | | | | | | |
| Novel gene fragment, 1324 bp, 76% SI to human | N/A | +1.5 | +1.5 | +1.3 | +1.4 | +1.3 | +1.5 | 04.04.01 | CITRIC ACID CYCLE | 21 | 24, 25 |
| flavoprotein subunit of complex II [D30648] | | | | | | | | | | | |
| Novel gene fragment, 852 bp, 81% SI to human vacuolar | N/A | +1.5 | +1.6 | +1.2 | +1.2 | +1.4 | +1.5 | 04.11.02 | PLASMA MEMBRANE SHUTTLING | 22 | 26, 27 |
| proton-ATPase subunit M9.2 [Y15286] | | | | | | | | | | | |
| Novel gene fragment, 178 bp, 91% SI to mouse myosin | N/A | +1.6 | +1.5 | +1.3 | +1.4 | +1.2 | +1.4 | 05.01.01.04 | STRUCTURAL ARM: HEAVY | 23 | 28 |
| light chain-2 isoform MLC-2a [S70785] | | | | | | | | | FILAMENTS | | |
| Novel gene fragment, 167 bp, 90% SI to mouse Rab20 | N/A | +1.7 | +1.8 | −1.2 | +1.0 | +1.2 | +1.9 | 08.01.03 | SYNAPTIC VESICLE COMPONENTS | 24 | 29 |
| [X80332] | | | | | | | | | | | |
| Novel gene fragment, 1070 bp, 80% SI to human muscle- | N/A | +1.5 | +1.7 | +1.3 | +1.2 | +1.2 | +1.5 | 09 | UNKNOWN FUNCTION | 25 | 30, 31, 32, |
| specific protein [AF249873] | | | | | | | | | | | 33, 34 |
| Novel gene fragment, 1143 bp, 82% SI to human | N/A | +1.7 | +1.8 | +1.2 | +1.3 | +1.4 | +1.7 | 09 | UNKNOWN FUNCTION | 26 | 35, 36 |
| sarcoma amplified sequence (SAS) [U01160] | | | | | | | | | | | |
| Novel gene fragment, 74 bp,75% SI to human secreted | N/A | +1.5 | +1.6 | +1.2 | −1.3 | +1.4 | +1.5 | 09 | UNKNOWN FUNCTION | 27 | 37 |
| protein [X30160] (from patent database)/[X97578] (from | | | | | | | | | | | |
| patent database)/potential cytokine) | | | | | | | | | | | |
| Novel gene fragment, 408 bp, 90% SI to human CGI-07 | N/A | +1.6 | +1.6 | +1.2 | −1.0 | +1.2 | +1.6 | 09 | UNKNOWN FUNCTION | 28 | 38, 39 |
| protein [AF132941] | | | | | | | | | | | |
| Novel gene fragment, 618 bp, 95% SI to rat progression | N/A | +1.5 | +1.6 | +1.4 | +1.0 | +1.0 | +1.5 | 09 | UNKNOWN FUNCTION | 29 | 40, 41 |
| related cDNA, ZNPE-120 3′ end partial sequence [patent | | | | | | | | | | | |
| database X90805] | | | | | | | | | | | |
| Novel gene fragment, 717 bp | N/A | +1.5 | +1.6 | +1.2 | +1.2 | +1.2 | +1.5 | 09 | UNKNOWN FUNCTION | 30 | 42, 43 |
| Novel gene fragment, 546 bp | N/A | +1.8 | +1.5 | +1.1 | +1.3 | −1.0 | +1.8 | 09 | UNKNOWN FUNCTION | 31 | 44, 45 |
| Novel gene fragment, 920 bp, 91% SI to human | N/A | +1.5 | +1.5 | +1.4 | +1.2 | +1.3 | +1.5 | 09 | UNKNOWN FUNCTION | 32 | 46, 47 |
| HSPC061 [AF161546] | | | | | | | | | | | |
| Novel gene fragment, 203 bp, 92% SI to rabbit | N/A | −1.7 | −1.7 | −1.4 | −1.2 | −1.1 | −1.7 | 09 | UNKNOWN FUNCTION | 33 | 48 |
| sarcoplasmic reticulum glycoprotein [J04480] | | | | | | | | | | | |
| Novel gene fragment, 178 bp, 68% SI to mouse IgG | N/A | +1.6 | +1.5 | +1.3 | +1.4 | +1.2 | +1.4 | 10 | INFLAMMATION | 34 | 49 |
| receptor (beta-Fc-gamma-RII)[M63159] | | | | | | | | | | | |
| Ribosomal protein L6 | X87107 | +1.6 | +1.5 | +1.2 | +1.4 | +1.4 | +1.6 | 01.02.01 | RIBOSOMAL PROTEIN | 35 | |
| CAP2 protein (adenylyl cyclase-associated protein 2) | U31935 | +2.0 | +2.0 | +1.4 | +1.4 | +1.2 | +2.0 | 02 | SIGNAL TRANSDUCTION | 36 | |
| Alpha-platelet-derived growth factor receptor | M63837 | +1.6 | +1.5 | +1.3 | −1.1 | −1.0 | +1.6 | 02.02.01 | TYROSINE KINASE RECEPTORS | 37 | |
| Rab GD1 alpha protein | X74402 | −1.6 | −1.5 | −1.3 | −1.1 | −1.1 | −1.6 | 02.07 | GTP/GDP EXCHANGE FACTORS | 38 | |
| PKC-zeta-interacting protein | Y08355 | +1.6 | +1.5 | +1.1 | −1.2 | +1.2 | +1.6 | 02.11.01 | SERINE/THREONINE KINASES | 39 | |
| ERK or MAP kinase | X65198 | +1.5 | +1.5 | +1.3 | +1.4 | +1.4 | +1.5 | 02.11.01 | SERINE/THREONINE KINASES | 40 | |
| Peroxisomal multifunctional enzyme type II | U37486 | +1.5 | +1.5 | +1.3 | +1.3 | +1.2 | +1.5 | 04.01.02.02 | PEROXISOMAL BETA OXIDATION | 41 | |
| HBP23 (heme-binding protein 23 kDa) | D30035 | +1.7 | +1.6 | +1.3 | +1.3 | +1.3 | +1.7 | 04.09 | DETOXIFICATION | 42 | |
| Caveolae-associated protein | U90725 | +3.0 | +1.5 | +1.3 | −1.0 | +1.3 | +3.1 | 07.01 | PLASMA MEMBRANE | 43 | |
| Prenylated rab acceptor 1 (PRA1) | AF025506 | +1.5 | +1.6 | +1.2 | +1.4 | +1.0 | +1.5 | 08.03.03 | SUBSTRATE/VESICLE SORTING | 44 | |

DEXFENFLURAMINE, FENFLURAMINE, DIHYDROERGOTAMINE MODULATED ONLY

| | | | | | | | | | | | |
|---|---|---|---|---|---|---|---|---|---|---|---|
| Novel gene fragment, 337 bp, 88% SI to rabbit cardiac | N/A | −1.7 | −1.6 | +1.3 | −1.3 | −2.5 | −1.5 | 02.03.02 | ION CHANNELS | 45 | 50 |
| ryanodine receptor (RyR-2)[U50465] | | | | | | | | | | | |
| Novel gene fragment, 81 bp, 85% SI to human titin | N/A | −2.2 | −1.8 | −1.1 | −1.3 | −2.8 | −2.2 | 05.01.01 | CYTOSKELETON COMPONENT | 46 | 51 |
| [X90568] | | | | | | | | | | | |
| Novel gene fragment, 428 bp, 86% SI to human titin | N/A | −2.0 | −1.8 | +1.1 | −1.1 | −1.7 | −2.0 | 05.01.01 | CYTOSKELETON COMPONENT | 47 | 52 |
| [X90568] | | | | | | | | | | | |
| Novel gene fragment, 374 bp, 88% SI to human titin | N/A | −2.3 | −1.8 | +1.1 | −1.2 | −5.5 | −2.3 | 05.01.01 | CYTOSKELETON COMPONENT | 48 | 53 |
| [X90568] | | | | | | | | | | | |
| Novel gene fragment, 428 bp, 85% SI to human titin | N/A | −2.2 | −1.9 | −1.3 | −1.3 | −3.8 | −2.2 | 05.01.01 | CYTOSKELETON COMPONENT | 49 | 54 |
| [90568] | | | | | | | | | | | |
| Novel gene fragment, 1216 bp, 93% SI to mouse | N/A | −1.9 | −1.7 | −1.2 | −1.1 | −1.5 | 1 | 05.01.01 | | 50 | 55, 56 |
| mircotubule-associated protein (MAP) 1B protein | | | | | | | | | | | |
| [AF115776] | | | | | | | | | | | |
| Novel gene fragment, 1115 bp, 83% SI to human | N/A | −2.0 | −1.8 | −1.4 | −1.4 | −1.5 | −2.1 | 09 | UNKNOWN FUNCTION | 51 | 57, 58, 59 |
| KIAA0549 protein [AB011121] | | | | | | | | | | | |
| Novel gene fragment, 153 bp | N/A | −1.7 | −1.7 | −1.4 | −1.2 | −1.7 | −1.7 | 09 | UNKNOWN FUNCTION | 52 | 60 |
| Novel gene fragment, 89 bp, 93% SI to human putative | N/A | −1.6 | −1.6 | −1.0 | +1.4 | +1.5 | +1.6 | 09.01.01.01 | CANCER | 53 | 61 |
| glialblastoma cell differentiation-related protein (GBDR1) | | | | | | | | | | | |
| [AF068195] | | | | | | | | | | | |
| Rho-associated kinase beta | U61266 | −1.9 | −1.8 | −1.2 | −1.1 | −2.6 | −1.10 | 02.11.01 | SERINE/THREONINE KINASES | 54 | |
| Adenylate kinase 3 | D13062 | +1.5 | +1.6 | +1.3 | +1.4 | +1.5 | +1.5 | 02.11.03 | NONPEPTIDES KINASES | 55 | |
| Amyloid beta-peptide binding protein | AF049878 | +1.5 | +1.6 | +1.3 | +1.3 | +1.7 | +1.5 | 04.01.02.01 | MITOCHONDRIAL BETA OXIDATION | 56 | |
| Mitochondrial adenine nuleotide translocator | D12771 | +1.5 | +1.6 | +1.4 | +1.4 | +1.8 | +1.5 | 04.04.03 | ATP/PROTON MOTIVE FORCE | 57 | |
| ALL SEROTONIN MODULATORS | | | | | | | | | INTERCONVERSION | | |
| Novel gene fragment, 710 bp, 94% SI to mouse chromatin | N/A | +3.8 | +3.4 | +2.8 | +7.5 | +2.8 | +3.0 | 01.01 | mRNA TRANSCRIPTION | 58 | 62, 63 |
| structural protein homolog Supt5 hp (Supt5 h)[U88539] | | | | | | | | | | | |
| Novel gene fragment, 1618 bp, 87% SI to mouse | N/A | −3.7 | −3.3 | −2.0 | −1.7 | −2.8 | −3.8 | 01.02.01 | RIROSOMAL PROTEINS | 59 | 64, 65 |
| mitochondrial genes coding for three transfer RNAs | | | | | | | | | | | |
| (specific for Phe, Val and Leu), 12S ribosomal RNA, and | | | | | | | | | | | |
| 16S ribosomal RNA [V00665] | | | | | | | | | | | |
| Novel gene fragment, 186 bp, 66% SI to human N- | N/A | −1.7 | −3.0 | −2.7 | −1.9 | −1.5 | −1.8 | 01.04.0 | GLYCOSYLATION | 60 | 66, 67 |
| acetylglucosaminyltransferase 1 (GlcNAc-TI)[M55621] | | | | | | | | | | | |
| Novel gene fragment, 238 bp, 95% SI to mouse MAP | N/A | −2.1 | −2.4 | −1.7 | −2.8 | −1.5 | −2.7 | 02.11.01 | SERINE/THREONINE KINASES | 61 | 68 |
| kinase-activated protein kinase 2 [X76850] | | | | | | | | | | | |
| Novel gene fragment, 173 bp, 70% SI to G protein- | N/A | −6.1 | −6.6 | −4.7 | −5.3 | −7.2 | −6.11 | 02.11.01 | SERINE/THREONINE KINASES | 62 | 69 |
| coupled receptor kinase GRK4 [X97568] | | | | | | | | | | | |
| Novel gene fragment, 133 bp, 77% SI to human apoptosis | N/A | −3.4 | −3.1 | −2.6 | −2.0 | −2.5 | −3.2 | 03.03.06 | CELL DEATH REGULATION | 63 | 70 |
| related protein hSARP3 [patent database: V19114] | | | | | | | | | | | |
| Novel gene fragment, 477 bp, 98% SI to peroxisomal | N/A | +5.0 | +4.8 | +3.7 | +7.0 | +8.0 | +5.1 | 04.01.02.02 | PEROXISOMAL BETA OXIDATION | 64 | 71, 72 |
| phytanoyl-CoA hydroxylase (PHYH) [AF121345] | | | | | | | | | | | |
| Novel gene fragment, 413 bp, 95% SI to mouse | N/A | +1.9 | +2.3 | +2.0 | +1.6 | +2.2 | +1.10 | 04.04 | OXIDATIVE PHOSPHORYLATION | 65 | 73 |
| dihydrolipoamide dehydrogenase (Dld) [U73445] | | | | | | | | | | | |
| Novel gene fragment, 726 bp, 76% SI to human | N/A | −4.1 | −4.0 | −4.4 | −3.0 | −3.6 | −4.1 | 04.04.01 | CITRIC ACID CYCLE | 66 | 74, 75 |
| succinate dehydrogenase flavoprotein subunit (SDH) | | | | | | | | | | | |
| [L21936] | | | | | | | | | | | |
| Novel gene fragment, 440 bp, 92% SI to mouse | N/A | +3.6 | +4.0 | +2.1 | +11.5 | +4.1 | +3.7 | 04.04.02 | ELECTRON TRANSPORT CHAIN | 67 | 76, 77 |
| cytochrome c oxidase Vllc [X52940] | | | | | | | | | | | |
| Novel gene fragment, 276 bp, 80% SI to human titin | N/A | −11.9 | −12.7 | −11.0 | −6.6 | −6.5 | −11.10 | 05.01.01 | CYTOSKELETON COMPONENT | 68 | 78 |
| [X90568] | | | | | | | | | | | |
| Novel gene fragment, 149 bp, 70% SI to human titin | N/A | −5.5 | −5.6 | −3.7 | −2.4 | +8.0 | −5.6 | 05.01.01 | CYTOSKELETON COMPONENT | 69 | 79 |
| [X90568] | | | | | | | | | | | |
| Novel gene fragment, 467 bp, 94% SI to mouse gelsolin | N/A | −3.2 | −3.6 | −3.8 | −1.7 | −1.6 | −3.2 | 05.01.03 | REGULATORS | 70 | 80, 81 |
| [J04953] | | | | | | | | | | | |
| Novel gene fragment, 535 bp, 90% SI to mouse | N/A | −2.0 | −3.7 | −2.0 | −1.9 | −1.7 | −2.1 | 05.01.03.03 | CONTRACTILE CA + 2 REGULATORS | 71 | 82, 83 |
| slow/cardiac troponin C [M29793] | | | | | | | | | | | |
| Novel gene fragment, 445 bp, 85% SI to human skeletal | N/A | −1.7 | −2.0 | −1.6 | −2.2 | −2.0 | 5.7 | 05.01.01.05 | BINDING PROTEINS | 72 | 84, 85 |
| muscle alpha 2 actinin [M86406] | | | | | | | | | | | |
| Novel gene fragment, 246 bp, 89% SI to mouse ponsin-1 | N/A | −3.1 | −2.8 | −2.5 | −2.8 | −3.5 | −3.2 | 05.03.01 | INTERFACE WITH CYTOSKELETON | 73 | 86 |
| [AF078667] | | | | | | | | | | | |
| Novel gene fragment, 126 bp, 77% SI to human DNA | N/A | +2.0 | +1.9 | +2.0 | +2.2 | +1.8 | +2.1 | 09 | UNKNOWN FUNCTION | 74 | 87 |
| sequence from cosmid V311G7, between markers | | | | | | | | | | | |
| DXS366 and DXS87 on chromosome X [Z69304] | | | | | | | | | | | |
| Novel gene fragment, 370 bp | N/A | −2.6 | −3.0 | −1.9 | −1.9 | −2.0 | −2.7 | 09 | UNKNOWN FUNCTION | 75 | 88, 89 |
| Novel gene fragment, 337 bp, 78% SI to novel human | N/A | −4.2 | −3.6 | −2.5 | −3.5 | −4.1 | −4.3 | 09 | UNKNOWN FUNCTION | 76 | 90 |
| protein AHNAK [M80899] | | | | | | | | | | | |
| Novel gene fragment, 100 bp, 93% SI to human | N/A | −7.5 | −9.0 | −7.6 | −8.4 | −9.3 | −9.3 | 09 | UNKNOWN FUNCTION | 77 | 91 |
| KIAA0750 protein [ABO18293] | | | | | | | | | | | |
| Novel gene fragment, 44 bp | N/A | −6.5 | −6.7 | −5.6 | −2.9 | −9.0 | −5.4 | 09 | UNKNOWN FUNCTION | 78 | 92 |
| Novel gene fragment, 698 bp, 93% SI to mouse plenty-of- | N/A | −2.3 | −2.5 | −2.3 | −2.3 | −1.9 | −2.3 | 09 | UNKNOWN FUNCTION | 79 | 93, 94 |
| prolines-101 [AF062655] | | | | | | | | | | | |
| Novel gene fragment, 660 bp, 84% SI to mouse | N/A | −3.6 | −3.7 | −4.1 | −1.9 | −3.0 | −3.8 | 09 | UNKNOWN FUNCTION | 80 | 95, 96 |
| membrane protein TMS-2 [AF181685] | | | | | | | | | | | |
| Novel gene fragment, 115 bp | N/A | −4.0 | −6.3 | −8.1 | −13.1 | −7.5 | −4.1 | 09 | UNKNOWN FUNCTION | 81 | 97 |
| Novel gene fragment, 294 bp, 95% SI to mouse Ndr1 | N/A | −2.0 | −2.0 | −2.5 | −3.3 | −3.3 | −2.0 | 09 | UNKNOWN FUNCTION | 82 | 98 |
| related protein Ndr2 [AB033921] | | | | | | | | | | | |
| Novel gene fragment, 198 bp | N/A | +2.5 | +2.7 | +2.5 | +3.1 | +2.6 | +2.5 | 09 | UNKNOWN FUNCTION | 83 | 99 |
| Novel gene fragment, 730 bp, 86% SI to mouse E800 | N/A | −2.0 | −2.4 | −1.8 | −2.1 | −1.7 | −2.8 | 09 | UNKNOWN FUNCTION | 84 | 100, 101 |
| [Y10968] | | | | | | | | | | | |
| Novel gene fragment, 294 bp, 98% SI to cysteine | N/A | −2.0 | −2.4 | −1.8 | −2.1 | −1.7 | −2.8 | | | 85 | 102 |
| conjugate beta-lyase [S61960] | | | | | | | | | | | |
| Aconitase | A1243266 | −3.1 | −3.4 | −3.9 | −2.9 | −3.2 | −1.9 | 04.04.01 | | 86 | |
| Ribosomal protein L7 | M17422 | +2.5 | +2.5 | +2.7 | +3.7 | +2.7 | +2.5 | 01.02.01 | RIBOSOMAL PROTEINS | 87 | |
| Ribosomal protein L9 | X51706 | +10.0 | +11.0 | +6.0 | +8.0 | +5.0 | +10.1 | 01.02.01 | RIBOSOMAL PROTEINS | 88 | |
| Ribosomal protein L12 | X53504 | −2.2 | −1.5 | −2.2 | −1.5 | −2.0 | −1.5 | 01.02.01 | RIBOSOMAL PROTEINS | 89 | |
| 18S, 5.8S, and 28S ribosomal RNA's | V01270 | +5 | +6 | +7 | +6 | +2.6 | +5 | 01.02.06 | RIBOSOMAL RNAs | 90 | |
| Pyruvate dehydrogenase kinase 2 (PDK2) | U10357 | −6.9 | −7.7 | −11.3 | −9.5 | −5.8 | −6.10 | 02.11.01 | SERINE/THREONINE KINASES | 91 | |
| D-Binding Protein (DBP) | J03179 | −1.9 | −1.9 | −1.5 | −1.7 | −1.5 | −1.4 | 02.14.01 | TRANSCRIPTION FACTORS | 92 | |
| Lipoprotein lipase | L03294 | −2.2 | −2.3 | −3.7 | −2.5 | −2.2 | −2.2 | 04.01.01 | FATTY ACID SYNTHESIS | 93 | |
| Non-neuronal enolase | X02610 | +3.5 | +3.5 | +3.1 | +3.1 | +3.1 | +3.5 | 04.03.01 | GLYCOLYSIS/GLUCONEOGENESIS | 94 | |
| Glycogen phosphorylase (muscle isozyme) | L10669 | −2.0 | −2.3 | −3.0 | −2.0 | −2.7 | −2.6 | 04.03.02 | GLYCOGEN MANIPULATION | 95 | |
| Cytochrome c oxidase subunit IV | X14209 | +10.0 | +10.0 | +8.0 | +8.0 | +4.1 | +10.0 | 04.04.02 | ELECTRON TRANSPORT CHAIN | 96 | |
| Alpha-globin | M17083 | +3.0 | +3.4 | +2.9 | +7.0 | +3.9 | +3.8 | 04.11.01 | EXTRACELLULAR TRANSPORT | 97 | |
| Beta-globin | X06701 | +3.1 | +3.6 | +4.0 | +5.0 | +3.3 | +3.11 | 04.11.01 | EXTRACELLULAR TRANSPORT | 98 | |
| Myoglobin | AF197916 | +4.7 | +4.0 | +2.2 | +7.0 | +3.9 | +4.7 | 04.11.01.01 | OXYGEN | 99 | |
| Titin | L38717 | −2.0 | −1.6 | +1.6 | +1.5 | −5.4 | −2.1 | 05.01.01 | CYTOSKELETON COMPONENT | 100 | |
| Skeletal muscle actin | V01218 | −18.2 | −14.9 | −13.1 | −3.8 | −14.3 | −18.3 | 05.01.01.03 | STRUCTURAL ARM: ACTINS & SHORT | 101 | |
| | | | | | | | | | FILAMENTS | | |
| Myosin light chain 2 (MLC2) | M11851 | +2.6 | +2.4 | +3.6 | +1.9 | −1.5 | +2.2 | 05.01.01.04 | STRUCTURAL ARM: HEAVY | 102 | |
| | | | | | | | | | FILAMENTS | | |
| Alpha cardiac myosin heavy chain | X15938 | −3.4 | −7.5 | −4.8 | −3.2 | −2.5 | −3.5 | 05.01.0l.04 | STRUCTURAL ARM: HEAVY | 103 | |
| | | | | | | | | | FILAMENTS | | |
| Troponin I | U77354 | +1.8 | +1.8 | +1.6 | +1.6 | +1.5 | +1.8 | 05.01.03.03 | CONTRACTILE CA + 2 REGULATORS | 104 | |
| Cardiac calsequestrin | AF001334 | −2.9 | −2.4 | −2.4 | −2.5 | −2.1 | −2.9 | 05.01.03.03 | CONTRACTILE CA + 2 REGULATORS | 105 | |
| Sulfated glycoprotein 2 | M16975 | −1.8 | −1.8 | −2.2 | −1.5 | −1.6 | −1.6 | 05.02 | EXTRACELLULAR MATRIX | 106 | |
| Aquaporin 7 | AB000507 | +1.5 | +1.5 | +1.8 | +1.6 | +1.7 | +1.5 | 07.01.03 | SURFACE STRUCTURES | 107 | |
| Carnitine/acylcarnitine carrier protein | X97831 | −2.5 | −2.6 | −2.6 | −1.9 | −2.8 | −2.5 | 07.05 | MITOCHONDRIAN | 108 | |
| Glu-Pro Dipeptide Repeat | U40628 | −5.0 | −5.6 | −3.1 | −5.5 | −5.1 | −6.2 | 09.01.02 | UNASSOCIATED | 109 | |
| Cystatin beta | X54737 | +2.5 | +2.7 | +2.5 | +3.1 | +2.6 | +2.5 | | | 110 | |

LL SEROTONIN MODULATORS EXCEPT SUMATRIPTAN

| | | | | | | | | | | | |
|---|---|---|---|---|---|---|---|---|---|---|---|
| Novel gene fragment, 593 bp, 90% SI to human | N/A | +1.9 | +2.1 | +2.1 | +2.1 | +1.7 | +1.10 | 02.12.01 | SERINE/THREONINE PHOSPHATASES | 111 | 103, 104 |
| calcineurin B-like protein [Z08983] | | | | | | | | | | | |
| Novel gene fragment, 179 bp, 88% SI to human titin | N/A | −3.1 | −3.0 | −1.8 | −2.0 | +6.0 | −3.1 | 05.01.01 | CYTOSKELETON COMPONENT | 112 | 105 |
| [X90568] | | | | | | | | | | | |
| Novel gene fragment, 700 bp, 91% SI to mouse periplakin | N/A | +2.5 | +2.0 | +2.1 | +2.2 | +1.7 | +2.5 | 05.10 | OTHERS/TISSUE ARCHITECTURE | 113 | 106, 107, 108 |
| (PPL) [AF116523] | | | | | | | | | | | |
| Long chain acyl-CoA dehydrogenase (LCAD) | J05029 | +1.7 | +1.8 | +1.6 | +1.8 | +1.5 | +1.7 | 04.01.02.01 | MITOCHONDRIAL BETA OXIDATION | 114 | |
| Sulfated glycoprotein 1 (SGP-1) | M19936 | −1.7 | −2.0 | −2.1 | −2.3 | −1.7 | −1.8 | 04.01.05 | GANGLIOSIDE BIOSYNTHESIS | 115 | |
| Catalase | M11670 | +2.1 | +2.1 | +1.9 | +1.8 | +1.6 | +2.1 | 04.09.02 | OXGEN RADICALS | 116 | |
| Alpha-fodrin (A2A) (Nonerythroid spectrin alpha subunit) | AF084186 | −1.7 | −1.8 | −1.6 | +5.0 | −1.5 | −1.7 | 05.01.01 | CYTOSKELETON COMPONENT | 117 | |
| Cardiac specific sodium channel alpha-subunit | M27902 | −2.0 | −2.1 | −2.1 | −2.9 | −1.6 | −2.0 | 07.01.01 | ION PUMPS | 118 | |
| Glu-Pro Dipeptide Repeat | U40628 | +4.6 | +4.2 | +2.7 | +6.0 | +1.9 | +4.2 | 09.01.02 | UNASSOCIATED | 119 | |
| ALL SEROTONIN REUPTAKE INHIBITORS | | | | | | | | | | | |
| Novel gene fragment, 200 bp, 64% SI to human KIAA0733 | N/A | −1.6 | −1.7 | −1.6 | −1.5 | −1.2 | −1.6 | 09 | UNKNOWN FUNCTION | 120 | 109 |
| protein [AB018316] | | | | | | | | | | | |
| RNA polymerase II transcription factor SIII (p18 subunit) | L42855 | +2.0 | +1.7 | +1.8 | +2.0 | +1.4 | +2.1 | 01.01 | mRNA TRANSCRIPTION | 121 | |
| Protein-tyrosine phosphalase (LRP) | L01702 | +1.7 | +2.0 | +1.5 | +1.5 | +1.4 | +1.8 | 02.12.02 | TYROSINE PHOSPHATASES | 122 | |
| Skeletal muscle selenoprotein W (SelW) | U25264 | −1.6 | −1.8 | −1.6 | −1.5 | −1.4 | −1.6 | 04.09 | DETOXIFICATION | 123 | |
| Sarcoplasmic reticulum 2+-Ca-ATPase | X15635 | −2.0 | −1.6 | −1.8 | −2.0 | −1.2 | −2.1 | 05.01.03.03 | CONTRACTILE Ca + 2 REGULATORS | 124 | |

ALL SEROTONON MODULATORS EXCEPT SUMATRIPAN AND FLUOXETINE

| | | | | | | | | | | |
|---|---|---|---|---|---|---|---|---|---|---|
| Ribosomal protein S7 | X53377 | +1.7 | +1.0 | +1.3 | +1.8 | +1.7 | +1.7 | 01.02.01 | RIBOSOMAL PROTEINS | 125 |
| Ribophorin I | X05300 | +2.0 | +1.6 | +1.4 | +1.6 | +1.5 | +2.0 | 04.11.02.09 | GLYCOPROTEINS | 126 |
| Beta cardiac myosin heavy chain | X15939 | +2.4 | +2.3 | +1.4 | −1.7 | −1.6 | +2.2 | 05.01.01.04 | STRUCTURAL ARM: HEAVY | 127 |
| | | | | | | | | | FILAMENTS | |
| DIHYDROERGOTAMINE MODULATED ONLY | | | | | | | | | | |
| ADP-ribosylation factor 1 | L12380 | +1.1 | +1.2 | −1.0 | −2.0 | +1.3 | +1.3 | 02.08 | SMALL GTP BINDING PROTEINS | 128 |

EROTONIN MODULATORS EXCEPT DIHYDROERGOTAMINE

| | | | | | | | | | | |
|---|---|---|---|---|---|---|---|---|---|---|
| Laminin receptor | U04942 | +2.6 | +2.6 | −1.5 | +4.6 | +1.1 | +2.6 | 05.03.02 | INTERFACE WITH EXTRACELLULAR | 129 |
| | | | | | | | | | MATRIX | |

LL SEROTONIN MODULATORS EXCEPT SIBUTRAMINE

| | | | | | | | | | | | |
|---|---|---|---|---|---|---|---|---|---|---|---|
| Novel gene fragment, 206 bp, 89% SI to human seryl- | N/A | +1.8 | +1.8 | +1.7 | +1.4 | +2.0 | +1.8 | 01.02.02 | AMINO ACYL tRNA SYNTHETASES | 130 | 110, 111 |
| tRNA synthetase [X91257] | | | | | | | | | | | |

DEXFENFLURAMINE, FENFLURAMINE, DIHYDROERGOTAMINE AND SUMATRIPTAN MODULATED ONLY

| | | | | | | | | | | |
|---|---|---|---|---|---|---|---|---|---|---|
| Annexin VI | X86086 | −10.0 | −1.8 | +1.3 | +1.4 | +3.5 | −09.0 | 09 | UNKNOWN FUNCTION | 131 |

DEXFENFLURARMINE, FENFLURAMINE, AND SUMATRIPTAN MODULATED ONLY

| | | | | | | | | | | | |
|---|---|---|---|---|---|---|---|---|---|---|---|
| Novel gene fragment, 325 bp, 90% SI to mouse N-RAP | N/A | +1.7 | +1.7 | +1.4 | −1.1 | +1.4 | +1.7 | 09 | UNKNOWN FUNCTION | 132 | 112, 113 |
| [U76618] | | | | | | | | | | | |
| Novel gene fragment, 337 bp | N/A | −2.0 | −2.4 | +1.0 | −1.1 | −1.2 | −2.1 | 09 | UNKNOWN FUNCTION | 133 | 114 |
| Novel gene fragment, 61 bp | N/A | −1.8 | −2.4 | −1.2 | +1.0 | −1.1 | −3 | 09 | UNKNOWN FUNCTION | 134 | 115, 116 |
| TATA-binding protein interacting protein 120 (TIP120) | D87671 | +2.3 | +1.9 | +1.4 | +1.3 | +1.1 | +2.4 | 02.14.01 | TRANSCRIPTION FACTORS | 135 | |
| Annexin VI | X86086 | −5.1 | −1.7 | | | | −5.1 | 09 | UNKNOWN FUNCTION | | |

FENFLURAMINE AND SUMATRIPTAN MODULATED ONLY

| | | | | | | | | | | |
|---|---|---|---|---|---|---|---|---|---|---|
| Long chain 3-ketoacyl-CoA thiolase | D16479 | −1.5 | −1.1 | −1.1 | −2.0 | −1.2 | −1.5 | 04.01.02.01 | MITOCHONDRIAL BETA OXIDATION | 136 |

FENFLURAMINE, SIBUTAMINE AND DIHYDROERGOTAMINE MODULATED ONLY

| | | | | | | | | | | |
|---|---|---|---|---|---|---|---|---|---|---|
| Thymosin beta-4 | M34043 | +1.4 | +1.6 | +1.4 | +1.5 | +1.6 | +1.4 | 5.01 | CYTOSKELETON | 137 |

DEXFENFLURAMINE, FENFLURAMINE, AND SIBUTAMINE MODULATED ONLY

| | | | | | | | | | | | |
|---|---|---|---|---|---|---|---|---|---|---|---|
| Novel gene fragment, 378 bp, 88% SI to human coatomer | N/A | −2.0 | −1.6 | −1.1 | −2.3 | +1.2 | −2.1 | 07.02.01 | LUMENAL PROTEINS | 138 | 117 |
| protein (COPA) [U24105] | | | | | | | | | | | |

DEXFENFLURAMINE, FENFLURAMINE, AND FLUOXETINE MODULATED ONLY

| | | | | | | | | | | |
|---|---|---|---|---|---|---|---|---|---|---|
| Serine protease | D88250 | +1.6 | +4.0 | +1.5 | +1.3 | +1.2 | +1.7 | 01.05.01 | PROTECLYSIS | 139 |
| MITOCHONDRIAL GENOME FRAGMENTS | | | | | | | | | | |
| Mitochondrial genome (bp 1127-1366) | X14848 | X | X | X | X | X | X | | | 140 |
| Mitochondrial genome (bp 1127-1183) | X14848 | X | X | X | X | X | X | | | 141 |
| Mitochondrial genome (bp 1144-1366) | X14848 | X | X | X | X | X | X | | | 142 |
| Mitochondrial genome (bp 1144-1482) | X14848 | X | X | X | X | X | X | | | 143 |
| Mitochondrial genome (bp 1679-1871) | X14848 | X | X | X | X | X | X | | | 144 |
| Mitochondrial genome (bp 2783-2990) | X14848 | X | X | X | X | X | X | | | 145 |
| Mitochondrial genome (bp 3444-3647) | X14848 | X | X | X | X | X | X | | | 146 |
| Mitochondrial genome (bp 3444-3679) | X14848 | X | X | X | X | X | X | | | 147 |
| Mitochondrial genome (bp 3444-3680) | X14848 | X | X | X | X | X | X | | | 148 |
| Mitochondrial genome (bp 3828-3936) | X14848 | X | X | X | X | X | X | | | 149 |
| Mitochondrial genome (bp 5322-5613) | X14848 | X | X | X | X | X | X | | | 150 |
| Mitochondrial genome (bp 5336-5613) | X14848 | X | X | X | X | X | X | | | 151 |
| Mitochondrial genome (bp 5337-5454) | X14848 | X | X | X | X | X | X | | | 152 |
| Mitochondrial genome (bp 5888-6041) | X14848 | X | X | | X | | | | | 153 |
| Mitochondrial genome (bp 6074-6159) | X14848 | X | X | X | | X | | | | 154 |
| Mitochondrial genome (bp 6247-6414) | X14848 | X | X | X | X | X | X | | | 155 |
| Mitochondrial genome (bp 6431-6603) | X14848 | X | X | X | X | X | X | | | 156 |
| Mitochondrial genome (bp 6503-6722) | X14848 | X | X | X | X | X | X | | | 157 |
| Mitochondrial genome (bp 6598-6838) | X14848 | X | X | X | X | X | X | | | 158 |
| Mitochondrial genome (bp 6598-6890) | X14848 | X | X | X | X | X | X | | | 159 |
| Mitochondrial genome (bp 6598-6890) | X14848 | X | X | X | X | X | X | | | 160 |
| Mitochondrial genome (bp 6598-6890) | X14848 | X | X | X | | X | | | | 161 |
| Mitochondrial genome (bp 6598-6895) | X14848 | X | X | X | X | X | X | | | 162 |
| Mitochondrial genome (bp 6598-6900) | X14848 | X | X | X | X | X | X | | | 163 |
| Mitochondrial genome (bp 6598-6909) | X14848 | X | X | X | X | X | X | | | 164 |
| Mitochondrial genome (bp 6613-6722) | X14848 | X | X | X | X | X | X | | | 165 |
| Mitochondrial genome (bp 6717-6872) | X14848 | X | X | X | X | X | X | | | 166 |
| Mitochondrial genome (bp 6717-6890) | X14848 | X | X | X | X | X | X | | | 167 |
| Mitochondrial genome (bp 6717-6895) | X14848 | X | X | X | X | X | X | | | 168 |
| Mitochondrial genome (bp 6717-6925) | X14848 | X | X | X | X | X | X | | | 169 |
| Mitochondrial genome (bp 7034-7240) | X14848 | X | X | X | X | X | X | | | 170 |
| Mitochondrial genome (bp 7474-7640) | X14848 | X | X | X | X | X | X | | | 171 |
| Mitochondrial genome (bp 7474-7642) | X14848 | X | X | X | X | X | X | | | 172 |
| Mitochondrial genome (bp 7474-7658) | X14848 | X | X | X | X | X | X | | | 173 |
| Mitochondrial genome (bp 7583-7679) | X14848 | X | X | X | X | X | X | | | 174 |
| Mitochondrial genome (bp 7812-7961) | X14848 | X | X | X | X | X | X | | | 175 |
| Mitochondrial genome (bp 7822-8249) | X14848 | X | X | X | X | X | X | | | 176 |
| Mitochondrial genome (bp 7956-8024) | X14848 | X | X | X | X | X | X | | | 177 |
| Mitochondrial genome (bp 7956-8302) | X14848 | X | X | X | X | X | X | | | 178 |
| Mitochondrial genome (bp 8269-8571) | X14848 | X | X | X | X | X | X | | | 179 |
| Mitochondrial genome (bp 8593-8810) | X14848 | X | X | X | X | X | X | | | 180 |
| Mitochondrial genome (bp 8593-8810) | X14848 | X | X | X | X | X | X | | | 181 |
| Mitochondrial genome (bp 8598-8921) | X14848 | X | X | X | X | X | X | | | 182 |
| Mitochondrial genome (bp 8593-8921) | X14848 | X | X | X | X | X | X | | | 183 |
| Mitochondrial genome (bp 8603-8921) | X14848 | X | X | X | X | X | X | | | 184 |
| Mitochondrial genome (bp 8603-8921) | X14848 | X | X | X | X | X | X | | | 185 |
| Mitochondrial genome (bp 8614-8810) | X14848 | X | X | X | X | X | X | | | 186 |
| Mitochondrial genome (bp 8819-8921) | X14848 | X | X | X | X | X | X | | | 187 |
| Mitochondrial genome (bp 8626-8921) | X14848 | X | X | X | X | X | X | | | 188 |
| Mitochondrial genome (bp 8628-8921) | X14848 | X | X | X | X | X | X | | | 189 |
| Mitochondrial genome (bp 8635-8921) | X14848 | X | X | X | X | X | X | | | 190 |
| Mitochondrial genome (bp 8780-8921) | X14848 | X | X | X | X | X | X | | | 191 |
| Mitochondrial genome (bp 8792-8921) | X14848 | X | X | | | | | | | 192 |
| Mitochondrial genome (bp 8899-9160) | X14848 | X | X | X | X | | | | | 193 |
| Mitochondrial genome (bp 8916-9057) | X14848 | X | X | X | X | X | X | | | 194 |
| Mitochondrial genome (bp 8916-9316) | X14848 | X | X | X | X | X | X | | | 195 |
| Mitochondrial genome (bp 8916-9361) | X14848 | X | X | X | X | X | X | | | 196 |
| Mitochondrial genome (bp 8962-9160) | X14848 | X | X | X | X | X | | | | 197 |
| Mitochondrial genome (bp 9221-9366) | X14848 | X | X | X | X | X | X | | | 198 |
| Mitochondrial genome (bp 9250-9368) | X14848 | X | X | X | X | X | X | | | 199 |
| Mitochondrial genome (bp 9253-9361) | X14848 | X | X | X | X | X | X | | | 200 |
| Mitochondrial genome (bp 9491-9763) | X14848 | X | X | X | X | X | X | | | 201 |
| Mitochondrial genome (bp 9910-10098) | X14848 | X | X | X | X | X | X | | | 202 |
| Mitochondrial genome (bp 10004-10090) | X14848 | X | X | X | X | X | X | | | 203 |
| Mitochondrial genome (bp 10855-10989) | X14848 | X | X | | | | | | | 204 |
| Mitochondrial genome (bp 11152-11501) | X14848 | X | X | X | X | X | X | | | 205 |
| Mitochondrial genome (bp 11230-11445) | X14848 | X | X | | | | | | | 206 |
| Mitochondrial genome (bp 11230-11506) | X14848 | X | X | X | X | X | X | | | 207 |
| Mitochondrial genome (bp 12937-12987) | X14848 | X | X | | | | | | | 208 |
| Mitochondrial genome (bp 14143-14441) | X14848 | X | X | X | X | | | | | 209 |
| Mitochondrial genome (bp 15861-16160) | X14848 | X | X | X | X | X | X | | | 210 |

Below follows additional discussion of nucleic acid sequences whose expression is differentially regulated in the presence of serotonin modulating agents.

CARDIOTOX1

CARDIOTOX1 is a novel gene fragment. The nucleic acid was initially identified in a cloned fragment having the following sequence:

| (SEQ ID NO: 1) | |
|---|---|
| 1 | ACTAGTGTCTTCCTCCGGTAGAGTTCTGGCAGGGGCGGGGTTCTTGGCTGTCCTGTGGCTGACGATGATGCTGCTGTTGG |
| 81 | TGACACGGGGACCATACCAGCCTTTCCAGAACTGTGTGTCCTTGCCCCCATGTTGAAAAAGGATGTGACGGACGCCAGGA |
| 161 | GGGTAATTGGAGAAGGTGTGGGAGATCT |

The cloned sequence was assembled into a contig resulting in the following 524 bp consensus sequence:

| (SEQ ID NO:2) | |
|---|---|
| 1 | TTTTTTTTTTTTTTTTTGATCTCCATCAAGCCAAAATAGGCTGGATTTACTGAAAACATTTATTACAACAAAATGTCAGC |
| 81 | GCTGTGTGACCGAGTTGATTTGGGCTTGACCAAAGTTGTATAGGGCAGGGGACCTACTCGTGGGACTGGGGACCTGACTG |
| 161 | CCCGCTAAGGGCTTAGGTCTTCCCAGGAGCCAAAGCTGAGTATCTTCCTCCTATTACTAGTGTCTTCCTCCGGTAGAGTT |
| 241 | CTGGCAGGGGCGGGGTTCTTGGCTGTCCTGTGGCTGACGATGATGCTGCTGTTGGTGACACGGGGACCATACCAGCCTTT |
| 321 | CCAGAACTGTGTGTCCTTGCCCCCATGTTGAAAAAGGATGTGACGGACGCCAGGAGGGTAATTGGAGAAGGTGTGGGAGA |
| 401 | TCTCTTGCCAGCTGGCGTCATTCCACTGTTCGATGGTCACAGGCGGAGGCTCAAAGGAGGCTAGGACAATGTAATCGGCA |
| 481 | AAGGCCAGCTGTACCCGGAGGTGATAGGTACAGCCGCAGTCTGC |

CARDIOTOX2

CARDIOTOX2 is a novel 306 bp gene fragment. The nucleic acid was initially identified in a cloned fragment having the following sequence:

| (SEQ ID NO:3) | |
|---|---|
| 81 | AGGAGAACCATCTGTGACTGAACTGGTCCAAGGGCAGGAAGGCCAGCAGTGGCTGAGGTTGCACTCCAGCTGGGAGAATC |
| 161 | TCAATGGGAGCACCCTGCAGGAGCTGCTGGTGCACAGGCGGTCCTGCCCAAGCGGAAGTGAGATTTCCCTTCTGTGTACC |
| 241 | AAGCAAGACTGTGGTCGCCGCCCTGCTGCCCGAATGAACAAGAGGATCCTTGGGGGTCGGACTAGT |

CARDIOTOX3

CARDIOTOX3 is a novel gene fragment. The nucleic acid was initially identified in a cloned fragment having the following sequence:

| (SEQ ID NO:4) | |
|---|---|
| 1 | TCATGAAGTGCGACATCGACATCAGGAAGGACCTGTACGCCAACAACGTCATGTCAGGGGGCACTACCATGTACCCCGGT |
| 81 | ATCGCTGACCGCATGCAGAAGGAGATCACAGCTCTGGCTCCCAGCACCATGAAGATCAAGATCATCGCCCCCCCTGAGCG |
| 161 | CAAGTACTCAGTGTGGATCGGCGGCTCCATCCTGGCCTCGCTGTCCACCTTCCAGCAGATGTCCATCACCCCGCAGGAGT |
| 241 | ACGACGAGGCCGGCCCCTCCATTGTGCACCGCAAATGCTTCTAGGCGCACCCGCGTCTGTGTACGCGCTCTCTCTCCTCA |
| 321 | GGACGACAATCGACCATCGTGCTATGGTTGCAGGGTGGCCCCATCCTCCGCCGTGGCTCCATCGCCGCCACTGCAGCCGG |
| 401 | C |

The cloned sequence was assembled into a contig resulting in the following 540 bp consensus sequence:

| (SEQ ID NO:5) | |
|---|---|
| 1 | TTTTTTTTTTTTTTTTTGGAGCAAAACAGAATGGCTGGCTTTAATGCTTCAAGTTTTCCATTTCCTTCCACAGGGCTTTG |
| 81 | TTTGAAAAATAACAAAATGAGGTAAAACGAGTGAATCTATGTACACGTCAAAAACAGGCGCCGGCTGCAGTGGCGGCGAT |
| 161 | GGAGCCACGGCGGAGGATGGGGCCACCCTGCAACCATAGCACGATGGTCGATTGTCGTCCTGAGGAGAGAGAGCGCGTAC |
| 241 | ACAGACGCGGGTGCGCCTAGAAGCATTTGCGGTGCACAATGGAGGGGCCGGCCTCGTCGTACTCCTGCTTGGTGATCCAC |
| 321 | ATCTGCTGGAAGGTGGACAGCGAGGCCAGGATGGAGCCGCCGATCCACACTGAGTACTTGCGCTCAGGGGGGGCGATGAT |
| 401 | CTTGATCTTCATGGTGCTGGGAGCCAGAGCTGTGATCTCCTTCTGCATGCGGTCAGCGATACCGGGGTACATGGTAGTGC |
| 481 | CCCCTGACATGACGTTGTTGGCGTACAGGTCCTTCCTGATGTCGATGTCGCACTTCATGA |

CARDIOTOX4

CARDIOTOX4 is a novel 80 bp gene fragment. The nucleic acid was initially identified in a cloned fragment having the following sequence:

| (SEQ ID NO: 6) | |
|---|---|
| 1 | CAATTGACAGAATCAGTGAGGTCCTCACTAGCCTCAGGATGTCCCAAAGTGCTGGCGAAGGAACCTCATCCAGCAAGCTT |

CARDIOTOX5

CARDIOTOX5 is a novel gene fragment. The nucleic acid was initially identified in a cloned fragment having the following sequence:

| (SEQ ID NO:7) | |
|---|---|
| 1 | ACTAGTGCTTCAATGTCAACCGAGAGTAAAATGTGTTTGTATGAAATGCCTCCATTTGACTAGATAGAGCTTTATTTGGA |
| 81 | GAAAGTCACATATAACATAATTGAACTTTGAATTATGCAATCCCGTGGATTTTAGAGTGCTCCTGGAGCAGGTGGCAGTC |
| 161 | ACCACTATCTACTTCCAGAACAGTCTCATCCTTTCCAGAAACCCACACTCTGTCTTTCCTCTATTCCAGATCT |

The cloned sequence was assembled into a contig resulting in the following 957 bp consensus sequence:

| (SEQ ID NO: 8) | |
|---|---|
| 1 | TTTGGAGCTGGGAACCGAACCCAGGGCCTTGTGCTTGCTAGGCAAGTGCTCTACCACTGAGCCAAATCCCCAACCCCTGT |
| 81 | AGTGCGCCTTCTATACTAGAAAGCTTGACCACTGAGCCACACCTCCCACTAGTGCTTCAATGTCAACCGAGAGTAAAATG |
| 161 | TGTTTGTATGAAATGCCTCCATTTGACTAGATAGAGCTTTATTTGGAGAAAGTCACATATAACATAATTGAACTTTGAAT |
| 241 | TATACAATCCCGTGGATTTTAGAGTGCTCCTGGAGCAGGTGGCAGTCACCACTATCTACTTCCAGAACAGTCTCATCCTT |
| 321 | TCCAGAAACCCACACTCTGTCTTTCCTCTATTCCAGATCTGTTAGACGAGTGGAATTACATAGTCCGGTCTTTTCTGAGT |
| 401 | TCTGTTACTAAGTTTTAAAGGTTTATTCTCAGGTAGCATCAGTCCGTAATGTATTACTGCTGAATAGTGTTCCGTGTATA |
| 481 | CAGACACCGTGTGTGTCTTCTTCCAGCGAGCAGAGGAACTCTGAGCTGTTTCTACTTTGGGGCTTTTGACTAATGCTATG |
| 561 | AACATCTGTGAAAAAGTTCGAAATGTTTGATTTAGTACAGACCCTAGTGGGGAGCTCCGGGGTCATATTATGACAGCCTC |
| 641 | AATTGTACTTCCTACAGTGGTTTTACCACCATTTCCTGCTCTCGTGNGATCTAGGCTCCAGCATCCCTCACAACTTTCTG |
| 721 | CCTGAGATGAAGAGGCATCTGATTGGGATCTTGGTTTGCATTTCCCTAATGTCTAATAATCTGAGCTTTTTTTCATGTGT |
| 801 | TCATTGGCTTTCTATGCTGCTTTGCAGAATGTTTATTTCAGGCTACAGTCTGCCTTTCAGCTGGGTTATCTTTCTGTTTT |
| 881 | TCTGTAGGATTTTTTATTTACGGTCAACTCATCTCTTAGATTAATTGGCATTTTTTTTTTCTCAACTTGCGGCCG |

CARDIOTOX6

CARDIOTOX6 is a novel 282 bp gene fragment. The nucleic acid was initially identified in a cloned fragment having the following sequence:

| (SEQ ID NO:9) | |
|---|---|
| 1 | TCCGGAAGATGCTCTACCCAACTCTGAGGTAATGAATGGGCCATTTACTTCTCCTCACTCTTCCCTGGAAATGCCTGCAC |
| 81 | CCCCACCAGCTCCTCGGACAGTCACAGATGAGGAAATGAATTTCGTTAAGACCTGTCTTCAGAGGTGGCGGAGTGAAATT |
| 161 | GAACAGGATATACAAGACTTAAAGAATTGTATCTCGAGCACCACCCAGGCTATTGAGCAGATGTACTGTGATCCTCTTCT |
| 241 | TCGTCAGGTGCCTTATCGCTTACATGCAGTTCTTGTTCATGA |

CARDIOTOX7

CARDIOTOX7 is a novel gene fragment. The nucleic acid was initially identified in a cloned fragment having the following sequence:

| (SEQ ID NO:10) | |
|---|---|
| 1 | CGTACCCCGCTCCACGTCCTGGCCACTCAGCCGGACATGGATGCCTTCCTTCAGGAGTGATCCGAACGCCATGTACTCTG |
| 81 | CCAGGGCCCAGTCCACAGTCCGGTTTGTCACAAGCTCTCTGCGAGTCTTCAAGATCCGGCTCAGCCCTCCATGGATGGTA |
| 161 | AAGTTCTCCACAGGTACAGAACTGGCCACATTCCCAATGTGGGTCAAGATGTCCTCCTCCAGGCCAGTGGAGGGGCAGGT |
| 241 | CATGCTCCTGGGCTGTCCATCCAGGGTGAAAAAGCCAGGCCAGGGGGAATCCAGCCAGTGCTTGATGTGCAAGATCT |

The cloned sequence was assembled into a contig resulting in the following 405 bp consensus sequence:

| (SEQ ID NO:11) | |
|---|---|
| 1 | CGGCCTGGTTAGGCCAAAGGTGGTTCATGGGGATGCAGGTTCTTTTGTCCACATTCTGGTCATGGAGCACATGGTGGCGA |
| 81 | TGGCTGAAGGTACCCCGCTCCACGTCCTGGCCACTCAGCCGGACATGGATGCCTTCCTTCAGGAGTGATCCGAACGCCAT |
| 161 | GTACTCTGCCAGGGCCCAGTCCACAGTCCGGTTTGTCACAAGCTCTCTGCGAGTCTTCAAGATCCGGCTCAGCCCTCCAT |
| 241 | GGATGGTAAAGTTCTCCACAGGTACAGAACTGGCCACATTCCCAATGTGGGTCAAGATGTCCTCCTCCAGGCCAGTGGAG |
| 321 | GGGCAGGTCATGCTCCTGGGCTGTCCATCCAGGGTGAAAAAGCCAGGCCAGGGGGAATCCAGCCAGTGCTTGATGTGCAA |
| 401 | GATCT |

CARDIOTOX10

CARDIOTOX10 is a novel gene fragment. The nucleic acid was initially identified in a cloned fragment having the following sequence:

| (SEQ ID NO:12) | |
|---|---|
| 1 | AGATCTTTCACAGACTTGTCATTCTTGTCAGCCTCTGCCTTTTGCCTTAAGGTTTCAATAATGGAGTGATCAGGGTTTAT |
| 81 | CTCCAGGTGTTTCTTTGCTGCCATGTAACCCATTGTTGAGTTGCCTCTGAGGGCTTGAGCTTTCATGA |

The cloned sequence was assembled into a contig resulting in the following 242 hp consensus sequence:

| (SEQ ID NO:13) | |
|---|---|
| 1 | AGATCTTTCACAGACTTGTCATTCTTGTCAGCCTCTGCCTTTTGCCTTAAGGTTTCAATAATGGAGTGATCAGGGTTTAT |
| 81 | CTCCAGGTGTTTCTTTGCTGCCATGTAACCCATTGTTGAGTTGCCTCTGAGGGCTTGAGCTTTCATGATTCTCTCCATGT |
| 161 | TTGCTGTCCAGCCATATGTGCTTGTGACAATACAGCATGGGGATGTCACCATTCGGTTTGACACAACCACCTTTTCAACC |
| 241 | TN |

CARDIOTOX11

CARDIOTOX11 is a novel 280 bp gene fragment. The nucleic acid was initially identified in a cloned fragment having the following sequence:

| (SEQ ID NO:14) | |
|---|---|
| 1 | TGTACATACCAGAGAGTTGATTGTGTGAAGAAGCTTCTAGAACTAGGAGCCAGTGTTGACCACGGTCGGTGGCTGGATAC |
| 81 | CCCACTGCATGCTGCAGCAAGGCAGTCCAGTGTGGAGGTCATCAATCTGCTCACTGAGTATGGGGCTAACCTGAAACTCA |
| 161 | GAAACTCGCAGGGCAAAAGTGCTCTTGAGCTCGCTGCTCCCAAAAGTAGTGTGGAGCAGGCACTCCTGCTCCATGAAGGT |
| 241 | CCACCTGCTCTTTCTCAGCTCTGCCGCTTGTGTGTCCGGA |

CARDIOTOX12

CARDIOTOX12 is a novel gene fragment. The nucleic acid was initially identified in a cloned fragment having the following sequence:

| (SEQ ID NO:15) | |
|---|---|
| 1 | GAATTCCAGAAGATCGCCATGGCCACAGCGATTGGATTCGCTATCATGGGGTTCATCGGCTTCTTTGTGAAACTGATCCA |
| 81 | CATCCCTATTAATAACATTATTGTGGGTGGCTGAGTCTTTGCTCATCGTGGGACTGGTGAACCAATGAGGGGGTGACAAG |
| 161 | CTCATGA |

The cloned sequence was assembled into a contig resulting in the following 348 bp consensus sequence:

| (SEQ ID NO:16) | |
|---|---|
| 1 | NCATCCAGGCAACTTTTACTTCATGAGCTTGTCACCCCCTCATTGGTTCACCAGTCCCACGATGAGCAAAGACTCAGCCA |
| 81 | CCCACAATAATGTTATTAATAGGGATGTGGATCAGTTTCACAAAGAAGCCGATGAACCCCATGATAGCGAATCCAATCGC |
| 161 | TGTGGCCATGGCGATCTTCTGGAATCTTTTCTATCAGGTTTGGTGCATCTTTTAACCAGCCGAATCGAGTCCTTTACAA |
| 241 | ACTGCCGACTTGGCTCGACAAACTGCATTACCTGATCCATGTTTGTGGGATGGCGGTTTGAGAGGGCAGAGACACGTAGC |
| 321 | CTAGGAGAGAATTGAGCCCAACGGAACN |

CARDIOTOX13

CARDIOTOX13 is a novel gene fragment. The nucleic acid was initially identified in a cloned fragment having the following sequence:

| (SEQ ID NO:17) | |
|---|---|
| 1 | TCTAGAGTCTTCCATCCAGGGTCTCCGGATAATGTGAAGCCGAGTGAGCCTCTGCCATCCAGCATGAAGAAACGGGACTG |
| 81 | AGCAGTCTGCCTGCCGTTCACATGGTGGTGAGGATCGCTGGCCCCAGGAAACACTGTCACACTGAAGCCACTAGCGTGTA |
| 161 | TCCGTGTGGATGTCGTGGGCGAAGCGTGGGATTTAGAGCAGCAGTGGTTTGTTTTGCTTTTTCTTTCATTTTGTTTTGTT |
| 241 | TTGTTTTGATTTTGCTATCTCATTCCATTTTTGACCAAAGCTTCTCTTTAAGTAGTTTATTATGGAAGATTGTCACACTA |
| 321 | ACTTAAAGGGGAAGGGACGTGTGTACA |

The cloned sequence was assembled into a contig resulting in the following 553 bp consensus sequence:

| (SEQ ID NO:18) | |
|---|---|
| 1 | TTTTTTTTTTTTTTTTTCACACTTGGGATTTTTCTTTAATTTTTTTAGCACACAATGTACACACGTCCCTTCCCCTTTAA |
| 81 | GTTAGTGTGACAATCTTCCATAATAAACTACTTAAAGAGAAGCTTTGGTCAAAAATGGAATGAGATAGCAAAATCAAAAC |
| 161 | AAAACAAAACAAAATGAAAGAAAAAGCAAAACAAACCACTGCTGCTCTAAATCCCACGCTTCGCCCACGACATCCACACG |
| 241 | GATACACGCTAGTGGCTTCAGTGTGACAGTGTTTCCTGGGGCCAGCGATCCTCACCACCATGTGAACGGCAGGCAGACTG |
| 321 | CTCAGTCCCGTTCCTCATGCTGGATGGCAGAGGCTCACTCGGCTTCACATTATCCGGAGACCCTGGATGGAAGACTCTAG |
| 401 | AGTCTTGAAATCCCAGATTGTCATGGCTCCATCGATGCCAGTAGTGCAAAATTTGCGACAATCTTGCTTGTCCACTTCAT |
| 481 | AAATAGACACTTGAGTGATGCTGTTCTGGTGCAGTGTTTCCAAGGCTGTGTTGCGGTCCTCGGTAGTGGCCCT |

CARDIOTOX19

CARDIOTOX19 is novel gene fragment. The nucleic acid was initially identified in two cloned fragments having the following sequence:

| (SEQ ID NO:19) | |
|---|---|
| 1 | AGATCTCTCCTAGCCAAGGGATGTTGAAACATGAAGGGTAAGGCCAGCCTGGTATCAGTTAAACTTACGACAAGGGAACA |
| 81 | AATACCAAGCTGGTGCTGTTGGTCTTATGGCTAGC and: |
| (SEQ ID NO:20) | |
| 1 | AGATCTGCCTAAAAAAGACTGCCCTGGGTGGTGAGCTAATGTCCATGACTTCTCTGGAAAGGTAGCCCTTTCTGGATTCT |
| 81 | GCCTACCTGGTCAGACACCAGGGGTTCTTTTTACAGCCAGAGAGACTCAACTCTAATGATATAGCTGGGGCAGTTACCCA |
| 161 | TACTCTCAGTCACCTGGGCTGTTCAAATGGTGACACTCTTCTAGGGCTGGGGACTGTGTCAAGGGAGTCCCAAGGAACTT |
| 241 | CTGGTCAGACATAGCCTCCTGTGATTTGGGGGTTCTTGGCTTGGCTGAAATCCTGTTATTTATTGCTTTGTTCCAGGGTG |
| 321 | GACTGTCAGGGCTTACTGCTTAACCTGTTTAAAATGAGGGACTTCAAGACTACACAGCATGGCTCTTTTCAGTTTATTGC |
| 401 | ATGAAGGAGTTACACTAGT |

The cloned sequence was assembled into a contig resulting in the following 1294 bp consensus sequence:

| (SEQ ID NO:21) | |
|---|---|
| 1 | TTTTTTTTTTTTTTTTTATTTCTGAAAACAAGCTTTATTTAAATAAGGATTTAAATACATTACATAACATTAAAACTGGA |
| 81 | AGGGAAAAGAAAACCAAAAGACCAGTTTGTTCCTTCACATGGCACTGGGCAGTGGCTTGTATTGTGTTGAAGCCTTTATA |
| 161 | GCTAGCCATAAGACCAACAGCACCAGCTTGGTATTTGTTCCCTTGTCGTAAGTTTAACTGATACCAGGCTGGCCTTACCC |
| 241 | TTCATGTTTCAACATCCCTTGGCTAGGAGAGATCTGCCTAAAAAAGACTGCCCTGGTGGTGAGCTAATGTCCATGACTTC |
| 321 | TCTGGAAAGGTAGCCCTTTCTGGATTCTGCCTACCTGGTCAGACACCAGGGGTTCTTTTTACAGCCAGAGAGACTCAACT |
| 401 | CTAATGATATAGCTGGGGCAGTTACCCATACTCTCAGTCACCTGGGCTGTTCAAATGGTGACACTCTTCTAGGGCTGGGG |
| 481 | ACTGTGTCAAGGGAGTCCCAAGGAACTTCTGGTCAGACATAGCCTCCTGTGATTTGGGGGTTCTTGGCTTGGCTGAAATC |
| 561 | CTGTTATTTATTGCTTTGTTCCAGGGTGGACTGTCAGGGCTTACTGCTTAACCTGTTTAAAATGAGGGACTTCAAGACTA |
| 641 | CACAGCATGGCTCTTTTCAGTTTATTGCATGAAGGAGTTACACTAGTCCAAGTTAAAAGCGGACCCCAAATGATTACATT |
| 721 | ATACAAGCTGTGAGGTTTTTAAACTTGTGACAAGGGACAGAAGGGAAATTCTACTCATTGCAAGGAAATCCTCACTTAAG |
| 801 | CTTCAGAGAGCCACAAGCACTTAAAACCCATGAACCTTCAGCTGATCGTCCTTAGCCAGTCCAATCTCTATCAGGAACTG |
| 881 | GCATATGTTCTTGCGCTGGTCACCCTGTAGCTGAATTACTTCTCCATATTCTGGATGCTCAATTACAGTACCATTGCAGG |
| 961 | CAAATTTCTTCTTAAACGCCTTCACTAGTTTCTTTTTATCGTAATCATCAGCGATCCCTTGGACAGTTGTAAGGGTCTTC |
| 1041 | CTGCCGTTTCTCTGTTGAATTCTTATATGGATATAATCCTCAGTGCCAGCAGGAAGCAGGTCATCACCCTTACTTGCATC |
| 1121 | AGCAAAGGGGTCGAAAGAGTGGAGGTTCTGGATAGCGGACATACGATACGATTCCTTTTCCTCGGTGGAAACGGCCTGCG |
| 1201 | GAAGGCGGCTGCGGGAGAAGGCGGGCGGGGGGGACGGAGCGTCGGGAAGCGAGGGGGCTCGAGGGGGAGGCAGCTGAGTC |
| 1281 | CTCGGCGGCGGCTC |

CARDIOTOX20

CARDIOTOX20 is a novel gene fragment. The nucleic acid was initially identified in a cloned fragment having the following sequence:

| (SEQ ID NO:22) | |
|---|---|
| 1 | GCTAGCAGCAATCACTTGGGGAAGAATCTGCAGTTGCTGATGGACCGGGTGGATGAAATGAGTCAGGACATAATCAAATA |
| 81 | CAACACATACATGAGGAACAGCAGTAAGCAGCAACAGCAGAAACACCAGTATCAGCAGCGTCGCCAGCAGGAGAATATGC |
| 161 | AGCGGCAGAGTCGAGGCGAGCCCCCGCTCCCTGAGGAGGACCTCTCCAAACTCTTCAAGCCCCACCAAGCCCCTGCCAGG |
| 241 | ATGGACTCGCTGCTCATTGCAGGCCAGATTAACACTTACTGCCAGAACATCAAGGAGTTCACTGCCCAAAACTTAGGCAA |
| 321 | ACTCTTCATGGCTCAGGCTCTTCAAGAATACAGTAACTAAGAAAAGGAAGCTT |

The cloned sequence was assembled into a contig resulting in the following 723 bp consensus sequence:

| (SEQ ID NO:23) | |
|---|---|
| 1 | TTTTTTTTTTTTTTTTTTTTTGAACAACCAAGTAACTTTTTATTATTGGTTATAAAGCCATTACAGCACTAAGAGCACAG |
| 81 | TGCGCCTCTCCACTTTGCAGTACAGAAACACATTTTCCAAGAGTCACTCTGGTGGAGTCTCAACAGTCTGTCTTCTTTGC |
| 161 | AGGAAGCTTCCTTTTCTTAGTTACTGTATTCTTGAAGAGCCTGAGCCATGAAGAGTTTGCCTAAGTTTTGGGCAGTGAAC |
| 241 | TCCTTGATGTTCTGGCAGTAAGTGTTAATCTGGCCTGCAATGAGCAGCGAGTCCATCCTGGCAGGGGCTTGGTGGGGCTT |
| 321 | GAAGAGTTTGGAGAGGTCCTCCTCAGGGAGCGGGGGCTCGCCTCGACTCTGCCGCTGCATATTCTCCTGCTGGCGACGCT |
| 401 | GCTGATACTGGTGTTTCTGCTGTTGCTGCTTACTGCTGTTCCTCATGTATGTGTTGTATTTGATTATGTCCTGACTCATT |
| 481 | TCATCCACCCGGTCCATCAGCAACTGCAGATTCTTCCCCAAGTGATTGCTGCTAGCAAGACTGAGCAATTCATGCTTATC |
| 561 | AGCCACAGCGGACTTCTTCTCAAGCTCCCACATCAGGACATTGGTCAAATGTGAGTTTTTAATTACAATCGGCACTTCTT |
| 641 | CAAACATGTGTTCAAAGGTGATGTTTGCCTTTTTCAATGCTTCCGGGGAAAAGTCCTTCTCTTTACAAACTTCCATCAGT |
| 721 | TTA |

CARDIOTOX21

CARDIOTOX21 is a novel gene fragment. The nucleic acid was initially identified in a cloned fragment having the following sequence:

| (SEQ ID NO:24) | |
|---|---|
| 1 | TGATCAACAGCTTGGCAGTACTTGATGTGAGGGACTCGAGTTGCACCATTGTCTCTCATTCTTGTGCAGTGATAAACTGG |
| 81 | TATAATTCTTAAATGATGTACAAACGAACAATCTTTTATTTCTAAATAAAACCACATAGTATTTGAGTTTAGTCCTATCT |
| 161 | ATTGGTCTGAAATATCAAATACAATTTTCTTCCCCTGTCTAGCTGAAGCAGTTGTGGTTTTCAAGTATTGTTTTGTTTAT |
| 241 | TCTCTGTGCCATATACTAAACTAGACTTTAAGGAATGTTAAAATGTAAATGGAAAATAGAGAAGTAGGGCAGGTCCTTAA |
| 321 | TAATTTGAAGCAAAGTTTGGATATGGTAAGTATCAAGCCAGTGCCTTGTTTAGGGGAGAGGTATTTGCATATGTCTACGT |
| 401 | ATATTTGATGGAGTATGTGCTGGCTAGC |

The cloned sequence was assembled into a contig resulting in the following 1324 bp consensus sequence:

| (SEQ ID NO:25) | |
|---|---|
| 1 | TTTTTTTTTTTTTTTTTCAAGTTTCAGAAGGGTTTATTTGACTTACAATTACTGGTTAAAGTCCTTCATTTCAAGGAAG |
| 81 | TCAGGGCAGGAACTTGAAGCAACTAGTTATACTCATGAATAAATGCATGCATGGAGAGTGCTCAGCTTGTTCTTATACAT |
| 161 | TCCAGATTCCTTTGTGTAGAGAATGGTGGTGCCCACAGTGGGCGGTCTTCCCTTCACAATTAACATAATCAAGCCAATCC |
| 241 | CTCTAAGACATGCCCAGGGACCAAGCTAACTGACACAATCCTGCACTGAGACCCTCTTCCTAGGTGATGCTAGATTGTGT |
| 321 | CAAGTTGACAAAGCTAGCCAGCACATACTCCATCAAATATACGTAGACATATGCAAATACCTCTCCCCTAAACAAGGCAC |
| 401 | TGGCTTGATACTTACCATATCCAAACTTTGCTTCAAATTATTAAGGACCTGCCCTACTTCTCTATTTTCCATTTACATTT |
| 481 | TAACATTCCTTAAAGTCTAGTTTAGTATATGGCACAGAGAATAAACAAAACAATACTTGAAAACCACAACTGCTTCAGCT |
| 561 | AGACAGGGGAAGAAAATTGTATTTGATATTTCAGACCAATAGATAGGACTAAACTCAAATACTATGTGGTTTTATTTAGA |
| 641 | AATAAAAGATTGTTCGTTTGTACATCATTTAAGAATTATACCAGTTTATCACTGCACAAGAATGAGAGACAATGGTGCAA |
| 721 | CTCGAGTCCCTCACATCAAGTACTGCCAAGCTGTTGATCATAATCTGTGAAGTGACTCCTTGTTCATGAGAGCAGATTTT |
| 801 | TAACAAGACGAGTATGAAAGGAAACCTAGGTAAGCTATGATGTATAATCACATAAGCTGGTCCTGTAGCTGTCAGGTTTT |
| 881 | TCAGTAGGAACGGATAGCAGGAGGTACAGTAGCACAGTCAGCCTCATTCAAGGTCTTGTCAATAACAGGTCTGTAATCCA |
| 961 | AAGTAACCTTCCCAGTCTTGGTGTCCACATATGAGAGGGTGTGCTTCCTCCAGTGTTCCGCAAATGGCTTCTTCTGCTGG |
| 1041 | CCCTCGATGGGCTTGGAGTAATCATACTCATCAATCCGCACCTTGTAATCTTCCCTGGCATGAGCTCCCCGTGACTCCTT |
| 1121 | CCGTGCTTCCGCACCATATATGGTCTGCAGTGCGCACAGCATCAGATTCTGCAGCTCCAGCGTCTCCACCAGGTCTGTGT |
| 1201 | TCCAGACCATTCCCCTGTCAAACGTCTTCAGATGCTGTAGGTCTCCATAGAGCTGGCTGACTTTTTCACAGCCTTCTTGC |
| 1281 | AGCACACTTCCCACACGGGACACGGCGGCATGGCTCTGCATCGA |

CARDIOTOX22

CARDIOTOX22 is a novel gene fragment. The nucleic acid was initially identified in a cloned fragment having the following sequence:

| (SEQ ID NO:26) | |
|---|---|
| 1 | TGTACATCTGCTGGGGTAGAGCTTCTCTCGAGCAGGCACTCCTGACTGTCCCACTGAGTCTCATTTGTCTTGCAGCAATT |
| 81 | CTTAAACACATCGCTGACTCTCATGTTGTGAGCAGGCAAGAGCCATATTCAAAGTGGCAGGCTTCAAGACAAGAGTAACA |
| 161 | GATTTCCCAGAACAGCACCTTTTCTCTCAGTCGAGTGCAGAGACACATCTCAAAGTCAGCTATGCAGGCACATAATTCAA |
| 241 | AGTGTAAAAAAGGTGAAGGAGAAAAAATACTGTATGCAGAGGAAGGCCTTCAAGTGTAAGGCAGGTAATGGCCGAAGTAG |
| 321 | GCTGTCGAGGAAGGAGGTCGGTGTGCAGGTGATTCTGTATCTAGA |

The cloned sequence was assembled into a contig resulting in the following 852 bp consensus sequence:

| (SEQ ID NO:27) | |
|---|---|
| 1 | TTTTTTTTTTTTTTTTTCCATAGAAAGAAGAAAAATAATTTATTCCAAAAGATGTAGAAGTAAGAAATTCATCCTGAAAA |
| 81 | TAGAGTTTGGTGTACATCTGCTGGGGTAGAGCTTCTCTCGAGCAGGCACTCCTGACTGTCCCACTGAGTCTCATTTGTCT |
| 161 | TGCAGCAATTCTTAAACACATCGCTGACTCTCATGTTGTGAGCAGGCAAGAGCCATATTCAAAGTGGCAGGCTTCAAGAC |
| 241 | AAGAGTAACAGATTTCCCAGAACAGCACCTTTTCTCTCAGTCGAGTGCAGAGACACATCTCAAAGTCAGCTATGCAGGCA |
| 321 | CATAATTCAAAGTGTAAAAAAGGTGAAGGAGAAAAAATACTGTATGCAGAGGAAGGCCTTCAAGTGTAAGGCAGGTAATG |
| 401 | GCCGAAGTAGGCTGTCGAGGAAGGAGGTCGGTGTGCAGGTGATTCTGTATCTAGAAGGCTTCTAGCTGTGACCTCAGTGC |
| 481 | CTGCACTGTGCAGCATGCCTTCATCCTCAAGGCCAGTGATACTTCAGATACCAGATGGTTTCATTTTTCAACTGTGGTCC |
| 561 | AAACAGAGGATTGAGCTGCGCCAGAATCGCAATCAGCCAAAAGAGATAGCAGCAAACGGAACAGGTCACCAACATGGTGA |
| 641 | TGATAACTCCCCGGTTAGGACCCTTGGGGATAAACCAGGGCACGAGGAGGCCCACGAAGCCCCAGAACACGCTCATCACG |
| 721 | ATCAAAGGCACAGTGAGGCCGTGGTATTCCATGCCTGCGACCCCGGAGCCGAACCAGTCCACCGCCTCACTCTCGTCCCA |
| 801 | CCCGGAAGTGTCAACAGAGGCTCACGTGACCGGCGCGCGAAAGCCCCACCCC |

CARDIOTOX23

CARDIOTOX23 is a novel 178 bp gene fragment. The nucleic acid was initially identified in a cloned fragment having the following sequence:

| (SEQ ID NO:28) | |
|---|---|
| 1 | GGATCGGGCACAGAGTTTATTGAGGTGACCCCAGTGTGTCTCTACTCCTCTTTCTCATCCCCGTGGGTGATGATGTAGCA |
| 81 | GAGAGACTTGTAGTCGATGTTGCCTGTCAGGTCCATGGGTGTCAGGGCGAACAGCTGCTCCACCTCAGCAGGAGAGAACT |
| 161 | TGTCTGCCTGGGTCATGA |

CARDIOTOX24

CARDIOTOX24 is a novel 167 bp gene fragment. The nucleic acid was initially identified in a cloned fragment having the following sequence:

| (SEQ ID NO:29) | |
|---|---|
| 1 | TCCGGAGGATGCGATGGCCCTTTACAAGAAGATCCTGAAGTACAAGATGTTAGACGAGAGGGAGATGCCGGGTGCCGAGC |
| 81 | AAATGTGCTTTGAGACCAGCGCCAAGACCGGACACAACGTGGACCTCCTCTTTGAAACCTTGTTCGACCTGGTGGTACCT |
| 161 | ATGATCA |

CARDIOTOX25

CARDIOTOX25 is a novel gene fragment. The nucleic acid was initially identified in four cloned fragments having the following sequences:

| (SEQ ID NO:30) | | |
|---|---|---|
| 1 | GGATCCGGGGTGTTAGGAGGAGTTGAGGGAGCTTGCTGTGAACCACCTTCCAGGTTACTTCCGTCAATTCTCCCATTCTG | |
| 81 | CATGGCAAGATTGTGATTGATTTGTGCTTTTGTTTCGTACTGGAAATTTTCAAAGGTGTATTTGTCAGATCTTCTTTGAC | |
| 161 | GCATCTTAAACAGTCTGGCACCGCGATTACCGAAATGGGACAATTCTTCTATCATGA | |
| (SEQ ID NO:31) | | |
| 1 | NAATTTCCTTCAGGGGTCCAGAATATCCTGGTGCAATGTTCTCCGGATTTGGGGGGCTTCGTGGATCC | |
| 1 | CCATGGACACGATGTCGACGGCATGGATCTGGGCAAGAAAGTTAGCGTCCCCAGAGACATCATGATAGAAGAATTGTCCC | (SEQ ID NO:32) |
| 81 | ATTTCGGTAATCGCGGTGCCAGACTGTTTAAGATGCGTCAAAGAAGATCT and: | |
| (SEQ ID NO:33) | | |
| 1 | TGATCACGACAGGAATATTCTCAGATATCCACCCCTTTGGTGTCCTATTAAAGCATCGTCTGCCCGAAAGAGGATTGGCA | |
| 81 | AAGGCCAAAAACCTGGGATCTGTTAGCAGCAGTCGTTCGAAGTCTGGAACCTTGAATTTAACCATTTTTGATGCTTTCTC | |
| 161 | AAAACCTCCAAATGGAGTGGCAACTCTGTTAAAGCTCCTGTAATCTGGCAGTTCTGCCTTTCCTTCAGGCTTGAAAAGTT | |
| 241 | TCGGGTACAAAGCTTCCAGGAGCTCTGGATCGTCGCCAATGGCCTGCTCCCAGGGAGACTGGTAGTACTTAGGAACAGCC | |
| 321 | GTCGTGTTAAATCTTTCAGGAGGAATTTCCTTCAGGGGTCCAGAATATCCTGGTGCAATGTTCTCCGGA | |

The cloned sequence was assembled into a contig resulting in the following 1070 bp consensus sequence:

| (SEQ ID NO:34) | |
|---|---|
| 1 | TTTTTTTTTTTTTTTTTGAGAGATTCTTAAACCAGAATTTAATTGTTCAGTTCAAATTGAACGCCACAAAATGAAATGTG |
| 81 | TGTAACCGCAATTGGATGACCACAGTGACGAGGCACTCAAATGGCTTCGCCGCTAAGAAGACCGACGGCAGCTTTTATGT |
| 161 | GTAGAGCTCTCGGCGGCCTGCCTGGCTTCCCGTTCACAAGTCATCTGACTCTGGCATAGTGACATCTTCTGCAGGCTCAG |
| 241 | TTGTGATCACGACAGGAATATTCTCAGATATCCACCCCTTTGGTGTCCTATTAAAGCATCGTCTGCCCGAAAGAGGATTG |
| 321 | GCAAAGGCCAAAAACCTGGGATCTGTTAGCAGCAGTCGTTCGAAGTCTGGAACCTTGAATTTAACCATTTTTGATGCTTT |
| 401 | CTCAAAACCTCCAAATGGAGTGGCAACTCTGTTAAAGCTCCTGTAATCTGGCAGTTCTGCCTTTCCTTCAGGCTTGAAAA |
| 481 | GTTTCGGGTACAAAGCTTCCAGGAGCTCTGGATCGTCGCCAATGGCCTGCTCCCAGGGAGACTGGTAGTACTTAGGAACA |
| 561 | GCCGTCGTGTTAAATCTTTCAGGAGGAAATTTCCTTCAGGGGTCCAGAATATCCTGGTGCAATGTTCTCCGGATTTGGGG |
| 641 | GGCTTCGTGGATCCGGGGTGTTAGGAGGAGTTGAGGGAGCTTGCTGTGAACCACCTTCCAGGTTACTTCCGTCAATTCTC |
| 721 | CCATTCTGCATGGCAAGATTGTGATTGATTTGTGCTTTTGTTTCGTACTGGAAATTTTCAAAGGTGTATTTGTCAGATCT |
| 801 | TCTTTGACGCATCTTAAACAGTCTGGCACCGCGATTACCGAAATGGGACAATTCTTCTATCATGATGTCTCTGGGGACGC |
| 881 | TAACTTTCTTGCCCAGATCCATGCCGTCGACATCGTGTCCATGGATTTCCTTCGTGATGGCTGAAGCTTGCTGTTTCCTT |
| 961 | TGCTTCACCATGGCACTGTGTGATAGCATAGTTTGTTTTTTGTTCCCTTGCTGTCAGACTGCACTTTTCAGCAGGGGTGA |
| 1041 | ATCCCAATTGCGGGGAGAGCTGGAAGTGTN |

CARDIOTOX26

CARDIOTOX26 is a novel gene fragment. The nucleic acid was initially identified in a cloned fragment having the following sequence:

| (SEQ ID NO:35) | |
|---|---|
| 1 | TGATCAGTTCTTAGGAGTGAGGTAAGGGACCTTTTTCTCTCTAAAACAAAAACCCCTTTTGGGGGTGGCCATCCTAGGTT |
| 81 | TCCAAGAATTTAGGAAGCCGGGAGAAGGGGAGGGCAAGTCAGAAGGATCACAAGGCTGGNTGAGTGTGGTGATGCCTGCA |
| 161 | ATACTGGCGGGAGGGTGAGGCAGGAGAATGCGGAGTTCAAGGCCCTCCATGGCTAGAGCTGGGTAGAAANGAGGCGAGGC |
| 241 | TGCAGGGATCCTGTCTGGGAGATCGAATCTCATAGAAGGGGACTAGGGTTGGCTCGAGGGTCTTTTTGATTCNGGA |

The cloned sequence was assembled into a contig resulting in the following 1143 bp consensus sequence:

| (SEQ ID NO:36) | |
|---|---|
| 1 | TTTTTTTTTTTTTTTTTGGTCTTTATTTTTCTTTAATGTTTTTCTGATTGGCGTTGCCACTGGGAGATTTGAAAAAGAAA |
| 81 | AAACCAAATGAAACAAGTTCCCTGCAAGGACCTAGGCAGGCAGTCCAGCTCTTTGGCTGACAAGATCGGAGAGGATCTTT |
| 161 | CAAATCCTTTCTTTGAATATTTGGTCAAAATGGCTTTAGTTTAAGTCCACTGGTCCTGTGAGATTGTAGGTGAGGCTGGG |
| 241 | ATGACAGACTGGTAGAAATACTTGCCCAGCACTTGTGAGGCCTTGGGTTGTAACTGGTTTTCCTTTGGTGTTCTGATTTT |
| 321 | GTTCTTGAAGGGAAGGAAAACAGTTATGAAAGGCTCCCATCAGCCACCTGTGCTTCTAGGAGTGCTAGACCCTCCTAGGC |
| 401 | AGAGAAATGGAGTCCTCTCCCCCTCCATAATATTCCCATCAAAATACACAGACATAAATAAATGTAGCCATCACTTGATC |
| 481 | AGTTCTTAGGAGTGAGGTAAGGGACCTTTTTCTCTCTAAAACAAAAACCCCTGTTGGGGGTGGCCATCCTAGGTTTCCAA |
| 561 | GAATTTAAAAAGCCGGGAGAAGGCAAGGCCAAGTCAGAAGGATCACAAGGCTGGCTGAGTGTGGTGATGCCTGCAATACT |
| 641 | GGCAGGAGGCTGAGGCAGGAGAATGCGGAGTTCAAGGCCCTCCATGGCTAGAGCTGGGTAGAAACCAGCCGAGGCTGCAA |
| 721 | AGATCCTGTCTGGGAGATCAAATCTCATAGAAAGGCACTAGGGTTGGCTCCAGGGTCTTTTTGATTCCGGAATCTCATTG |
| 801 | CTAGCCAAACACCGAGGATCTCTGTGAAACTGAAGAAGAGCCCAACACCTCCTAGGATCTTGAGAGCTTTGTCTGAATGT |
| 881 | TTTAGGAATGTCTCCCCACACATCTGGCATGGAGAGCTCCTAGTTTTGCACAGTGCACTGCAGGAAGCATCATCATGTAG |
| 961 | GTGCACGGTTGTAAGGTTAAACAAACCACAGCAGTCAAAACTTCTCTCCAGTTCATGCCTGGTACTGTTGCTCAAGACCC |
| 1041 | ACCACGAAGCATTGATGACATCTGCCTGTGTGTTTCTGTTAAGAGCCAGACATGAGCAAGAGATTCCAAACTGGAAGATG |
| 1121 | AAGACCAAACCCAGGATGATCAN |

CARDIOTOX27

CARDIOTOX27 is a novel 74 bp gene fragment. The nucleic acid has the following sequence:

| (SEQ ID NO:37) | |
|---|---|
| 1 | GTGCACTCTGCAGTGAGGACAATAGATGGCTCACTGTGGCAGCCTGGCTGAGAGGGAACTCTCATGCTGCTAGC |

CARDIOTOX28

CARDIOTOX28 is a novel gene fragment. The nucleic acid was initially identified in a cloned fragment having the following sequence:

| (SEQ ID NO:38) | |
|---|---|
| 1 | AGATCTCGGACTATGCTGCATTCTATCACAATAAATTCTTCTAGCTGTTTAGGATGGCATAAACTATTGAAAGGATGACT |
| 81 | CCAGAAGGTGTTCCCATCAATGTCTGCAACTTGTAAGGTATTTGGGTCTATGAGATGGATGGCACTAGT |

The cloned sequence was assembled into a contig resulting in the following 408 bp consensus sequence:

| (SEQ ID NO:39) | |
|---|---|
| 1 | TCATGATGGTCTGGATTTTTATTATTCTTCAAAACAGCATGCTCAGAAGATGGTGGAGTTTCTTCAGGGTACAGTTCCCT |
| 81 | GTAGATACAAATCATCACAAAGATTGATCTCCCAGGATATTCATAGTAACACATACAATTACAAGAGTACTTTTTCTGTG |
| 161 | GAAATTGTTCTAATATGCAAGGATAATGTTGTCTGTCTGTCACCAAAACTGGCACAGAGCCTTGGAAATATGAACCAGAT |
| 241 | ATGTATTTGTATACGAGTAACTAGTGCCATCCATCTCATAGACCCAAATACCTTACAAGTTGCAGACATTGATGGGAACA |
| 321 | CCTTCTGGAGTCATCCTTTCAATAGTTTATGCCATCCTAAACAGCTAGAAGAATTTATTGTGATAGAATGCAGCATAGTC |
| 401 | CGAGATCT |

CARIDOTOX29

CARDIOTOX29 is a novel gene fragment. The nucleic acid was initially identified in a cloned fragment having the following sequence:

| (SEQ ID NO:40) | |
|---|---|
| 1 | GTGCACGCCTTCGACATGGAGGATCTGGGGGATAAGGCCGTGTATTGCCGGTGCTGGAGGTCTAAAAAGTTCCCGTTCTG |
| 81 | CGATGGGGCTCACATAAAGCACAATGAGGAGACTGGAGACAACGTGGGACCTCTGATCA |

The cloned sequence was assembled into a contig resulting in the following 618 bp consensus sequence:

| (SEQ ID NO:41) | |
|---|---|
| 1 | TTTTTTTTTTTTTTTTTGATTTTGGAATAATTTAATATATAACCTCAAGACATAACTCTATTCTAAGACCATTATTTTAA |
| 81 | AGGAACGGATCCTTACGAGACCAAGATAACCCACAGAGCATGAGGTTGGTTCAGCCTTTCCTTTTCTTCTTCTTTCAACA |
| 161 | AATGTGCACCACGATGTTTCAATGGCAAGGCCGATGCCGTGAACATGAAAGCTGCGATTTGCAAGTACCAACCACACCAG |
| 241 | AACCTGGGAGGCCAACCAGACAGTGGGTTGGGTGCCATTCTAATTAAATGATCAGGTGACATCACAACACGCTGGGGTGT |
| 321 | AGCCTCGCAACTGTCCATTAAGTTTCTTTTTTCTTGATGATCAGAGGTCCCACGTTGTCTCCAGTCTCCTCATTGTGCTT |
| 401 | TATGTGAGCCCCATCGCAGAACGGGAACTTTTTAGACCTCCAGCACCGGCAATACACGGCCTTATCCCCCAGATCCTCCA |
| 481 | TGTCGAAGGCGTGCACAACCTTCGGGTTGTCTTTCTGGATCTGAAGGTTCACCATAGCTTTGGTGCGACTCTCTTTAGCG |
| 561 | TAGAACTTCTTGTAAGCCAGGTAACCGATAACGGCTGTGCCAGCAGCAAAGGTCACGG |

CARDIOTOX30

CARDIOTOX30 is a novel gene fragment. The nucleic acid was initially identified in a cloned fragment having the following sequence:

| (SEQ ID NO:42) | |
|---|---|
| 1 | GTGCACCCTTACATCAGAACAAAAGCTACTTTGAGTTCAAAATCCAGTCTACCGGAATCTGGGGTATAGGTGTTGCAACT |
| 81 | CAGAAAGTTAACTTGAACCAGATTCCTCTTGGCCGTGACATGCATAGCCTGGTGATGAGAAATGATGGAGCCCTGTACCA |
| 161 | CAACAACGAAGAGAAAAACAGGCTGCCAGCAAACAGCCTTCCTCAGGAGGGAGATGTAGTGGGTATAACATATGACCATG |
| 241 | TAGAATTAAATGTATATTTGAATGGGAAAAACATGCATTGTCCAGCATCAGGTATACGAGGGACCGTGTATCCAGTCGTG |
| 321 | TATGTTGACGACAGTGCAATTTTGGATTGCCAGTTCAGTGAATTTTATCATACTCCTCCACCTGGTTTTGAAAAAATACT |
| 401 | ATTTGAGCAGCAGATCT |

The cloned sequence was assembled into a contig resulting in the following 717 bp consensus sequence:

| (SEQ ID NO:43) | |
|---|---|
| 1 | TTTTTTTTTTTTTTTTTGTCAAACAAATACTTTTTATAAGAAAAATTCCCTTTAAATATTTATATACATGTTACCACGTA |
| 81 | ATACTGTTAATCAAACCCATGGTTTATTTGTTTAAATAAGATTAAATAAATTGCCTAGATCTTTTAAATCAAACCTTAGT |
| 161 | ATGGTATAATGGATATATGGGTTCCTTAGACAACAATAAGAAGCATGTGTTCTTGTCTCTAGATCAAGGAGAGCTTTATC |
| 241 | AAGTGGTAAGCGCTGTGTGATGGTGCAGAAGTCTAAGTTTTGAAAACAAACTCATTCAGAAGATCTGCTGCTCAAATAGT |
| 321 | ATTTTTTCAAAACCAGGTGGAGGAGTATGATAAAATTCACTGAACTGGCAATCCAAAATTGCACTGTCGTCAACATACAC |
| 401 | GACTGGATACACGGTCCCTCGTATACCTGATGCTGGACAATGCATGTTTTTCCCATTCAAATATACATTTAATTCTACAT |
| 481 | GGTCATATGTTATACCCACTACATCTCCCTCCTGAGGAAGGCTGTTTGCTGGCAGCCTGTTTTTCTCTTCGTTGTTGTGG |
| 561 | TACAGGGCTCCATCATTTCTCATCACCAGGCTATGCATGTCACGGCCAAGAGGAATCTGGTTCAAGTTAACTTTCTGAGT |
| 641 | TGCAACACCTATACCCCAGATTCCGGTAGACTGGATTTTGAACTCAAAGTAGCTTTTGTTCTGATGTAAGGGTGCAC |

CARDIOTOX31

CARDIOTOX31 is a novel gene fragment. The nucleic acid was initially identified in a cloned fragment having the following sequence:

| (SEQ ID NO:44) | |
|---|---|
| 1 | AGATCTAACTACTCCAACCTTCACAATTCCAGCTACTTGATAATAATAGGAGTAACCCAATGAATACTGTATGGTCTGAA |
| 81 | AGCTACTATACAATATGATTCTTGAGGAGGAGGGAGAGAGGGAGAGAGGGAGTTAGAGACTGTCACAAAGCCCTGGGTGC |
| 161 | TTCTCTGGAGTTAGCAGGGAAACAGGACCCTGGGCAAGCAGCTCGGGTGCCCTAGG |

The cloned sequence was assembled into a contig resulting in the following 546 bp consensus sequence:

| (SEQ ID NO:45) | |
|---|---|
| 1 | TTTTTTTTTTTTTTTTTGGTGTTTCTCTCTTTTATTTAAAAACAGTGCTTCGTTACCATTTGCAAAGGCTGAGGCAGGGC |
| 81 | CCCTCCTTTGCTAAGAGTTTATAAAAGCCAGCAACATGATCAATAATTTATACACATGGAGAGTAATACAAAAAATAATG |
| 161 | AATAAAAGCTAAAGATCTAACTACTCCAACCTTCACAATTCCAGCTACTTGATAATAATAGGAGTAACCCAATGAATACT |
| 241 | GTATGGTCTGAAAGCTACTATACAATATGATTCTTGAGGAGGAGGGAGAGAGGGAGAGAGGGAGTTAGAGACTGTCACAA |
| 321 | AGCCCTGGGTGCTTCTCTGGAGTTAGCAGGGAAACAGGACCCTGGCCAAGCAGCTCGGGTGCCCTAGGAGGTGACTCTGG |
| 401 | GAGAGGATGGGAAGGAAGGAGACACAGCTGGGTGGTCAATTGGACAAGCATTCCAGTATGCCCCCATGTCCCAGAGGTAC |
| 481 | CTGTCCTGCCACAGGGAAACCACACGTGCTAGGCAAGCCACTCCCTGCCACAGAGGTGTGGAGGAG |

CARDIOTOX32

CARDIOTOX32 is a novel gene fragment. The nucleic acid has the following sequence:

| (SEQ ID NO:46) | |
|---|---|
| 1 | TGTACAAGAGAAGGACTAAGAACCAAACTGTTTACAGAGATCCAAGCACGAGTGAGAGAGCACACTCCTCACACGGCTTT |
| 81 | CCGATGATACTCAGGAGGAGCCACTTCATAATCACTGGCACTGAACAGAGTTGCAGAATTCTTTGCCAGGTACTTGAGGA |
| 161 | AATCATGTAGATAGTTCAGTAATAAAGCAAGGCTTTTCTCATCTAGA |

The cloned sequence was assembled into a contig resulting in the following 920 bp consensus sequence:

| (SEQ ID NO:47) | |
|---|---|
| 1 | TTTTTTTTTTTTTTTTTTGAAATTTAAAGAAAAATTTATTGAAGATCTGAAAAACAACTCCTACAAGATTGACTTTTCCA |
| 81 | TAAAACTGTAGCTACACGATGCATTGCGTCTATCATGTTAAAACGTGCATTAGACACAAATACAAAAACCATGAAAACAA |
| 161 | GCCACCATTCTTTAACAATTGAGCAAAGATAAAATGCCTAAGGAACAACATGGATGACTTGCAAAGGATGGGCTCTTTAA |
| 241 | GCACCATTTAAAAAAAAAAAGAGCACAGATGGATGAGTGTTCAGTTATACACACTGAAGGGAACCTTTGGCACTAGGAGT |
| 321 | CAGAGCATTTTGTCATAGAGCATTAACACATATTATAAAAGTGCGTAGTGTCAAAGGAACAGAACCACCAGCATTCAAAA |
| 401 | GCAGCTTTGTCAACTAGGCAAACACTCTACAGCATGTCTCTCCGTTGTCCATCACTGATACACTGGTAGAAACTTTGAAA |
| 481 | TGAAAAAAAGAAAGAAAAAAGGAGCAGTTAACTCCTTTTATTTTCTCTGTTTAAAATCAAACAGGAAACAAACATCAACT |
| 561 | CTGTTATACACTAACGGTCTTCAAAGTACATCATTTGTACAAGAGAAGGACTAAGAACCAAACTGTTTACAGAGATCCAA |
| 641 | GCACGAGTGAGAGAGCACACTCCTCACACGGCTTTCCGATGATACTCAGGAGGAGCCACTTCATAATCACTGGCACTGAA |
| 721 | CAGAGTTGCAGAATTCTTTGCCAGGTACTTGAGGAAATCATGTAGATAGTTCAGTAATAAAGCAAGGCTTTTCTCATCTA |
| 801 | GAGGTGTATAGGCCAACATCGCTCCAATTCGCACAAACAATCTCAGTAAGTGTGGCGCTCCATACACCTGGGACATGGGT |
| 881 | GCATCCGGGTGATCGGCCAAAATTTCAGCATACTGTGGTC |

CARDIOTOX33

CARDIOTOX33 is a novel 203 bp gene fragment. The nucleic acid was initially identified in a cloned fragment having the following sequence:

| (SEQ ID NO:48) | |
|---|---|
| 1 | AGATCTCTCTCCTGGAAGACCTGAACCAGGTGATAGAGAACAGGCTCGAGAACAAGATTGCTTTTATTCGCCAGCACGCC |
| 81 | ATCAGGGTCCGAATCCACGCCCTTTTAGTTGACCGCTATCTGCAGACTTACAAGGACAAAATGACCTTCTTCAGTGACGG |
| 161 | GGAACTGGTCTTTAAGGACATTGTGGAAGATCCTGATAAATTC |

CARDIOTOX34

CARDIOTOX34 is a novel 178 bp gene fragment. The nucleic acid was initially identified in a cloned fragment having the following sequence:

| (SEQ ID NO:49) | |
|---|---|
| 1 | GGATCCCACGCCCTCTTCTGAGGGTACTAGACATGCACACCGTGTGCAGACATGCATGCAGGTAAAATGTGTGCTCACAA |
| 81 | AACTAAAAACCTGAAAAAGAAAACCAACCCTGCATTTGTGGAGTCATCACAGCCCATAGACTGTGCCAACGAGTGTGTGA |
| 161 | ACCAGAAGAGAAGTTCATGA |

CARDIOTOX45

CARDIOTOX45 is a novel 337 bp gene fragment. The nucleic acid was initially identified in a cloned fragment having the following sequence:

| (SEQ ID NO:50) | |
|---|---|
| 1 | TCCGGATGAGCAACCTCACCACAACATTTGCATTCTCTTCCACACTCTCCCCATTACAGAAGACGGCAAATCTGAGAAAG |
| 81 | TCAAGATATCGTTCTCCTTCAACTGGATTCCACCCAATGTCTGGGTAACCCTTAGACACCAGCATCTGGCAGCTCTGCAG |
| 161 | ACCACAGCCGGCCAGATAGCGAACCACCTTCTCCAGATCCGGCTCTCGTAGAGCAAGGGCAAGCTCATTGTTATCCATCA |
| 241 | CTGACGCTGCGGCCACGTCTAATGGAGTTGAACCTCTCATGGCTGGTGAGGCAAGACCAACACTGCTGTTTTCCAGTAAA |
| 321 | TAACTGAGATGATCA |

CARDIOTOX46

CARDIOTOX46 is a novel 81 bp gene fragment. The nucleic acid was initially identified in a cloned fragment having the following sequence:

| (SEQ ID NO:51) | |
|---|---|
| 1 | GAATTCTGCGTCAGTCCAGAGACAGTGAATTGAGTCTCGATAACATTGGTGAAGCTGGCCTTAGTCCACCTCCCATCCGG |
| 81 | A |

CARDIOTOX47

CARDIOTOX47 is a novel 428 bp gene fragment. The nucleic acid has the following sequence:

| (SEQ ID NO 52) | |
|---|---|
| 1 | TCCGGATGTTAGTTTTGTCTTGACAGACATAGCTGTTCTCCGTGGTCGGCTGAGCCCAGTCTCGTTCTCAGCAAATACTC |
| 81 | GGAACTCATATTCAGTTGCTTCTAGCAAACCTCCGATGGTGAACTGCCTGTCCTTGATCCGTTCCTTATTGCTCTTCTTC |
| 161 | CAAGCACTGTCCCCAGACTGTCTGTACTCAACCCAGTAGCCAAGGATTTCTTTGCCACCATCGCATTCGGGCTTCTCCCA |
| 241 | CTGGAGGATGACACTGTCTTTGGATATCGAAAGAATCTCGAGTTCTCCTGGTTGGCTTGGCTTATCGAAGGGATCTTTGC |
| 321 | AAACGACGGGTTCAGAAGCAGGGCTGGTCTCGCTAAGGCCCACGTCATTCTGTGCGATGATACGGAATTGATATTCTGCG |
| 401 | TCAGGAACAAGGCCAGTGACCGTGTACA |

CARDIOTOX48

CARDIOTOX48 is a novel 374 bp gene fragment. The nucleic acid has the following sequence:

| (SEQ ID NO:53) | |
|---|---|
| 1 | GGTACCATTTTACATTTGCTTTCTCTCTGGAGAGCTGGCAGGAGAAGACAGCGTCGTCAAACTCTGTGACCGTCTGGTCT |
| 81 | TCCAGGTGCTCCACGAATTCCGTTGGGGCTTCGATGATGAGCAGCTCTGCCACGGATTTATCTTGACCAGCAGTAACGAT |
| 161 | GTATCCATCTTCATCTGGGAAGCCACAGTCCTTGATGATTAGAGAGTGCTTGTACTTGTCAATGCGGTATGATATACGGT |
| 241 | TGTCAAAGCCACTTCTTCCCCATTTTTGGTCCACTTCAGGGTTACATTGAGACGATTCACCTTGCACCAGAAACGTGACT |
| 321 | GACTTCTTCTCCATTGTTTCAATATCTTTAAGGGGTTCGATAATCCTAAGATCT |

CARDIOTOX49

CARDIOTOX49 is a novel 429 bp gene fragment. The nucleic acid was initially identified in a cloned fragment having the following sequence:

| (SEQ ID NO:54) | |
|---|---|
| 1 | ACTAGTCACCTCGATCTGGGCTCTCTCCGTGAGAATGCCTTCAGCCTTTTCCCACTTCACCTCAGGTTCTGGGCGACCTT |
| 81 | TGATAGTGACAAACAGGCGCAAAGTGGCACTTGCCCTCAGAGTGACCACCTTCCTGAGATCAGCATCGAGTTCTATTTCT |
| 161 | GGGGCTTCCATCCTCTCCTGAGCCACAACAGAGCCTGGTATAGTTGCAGGCTCACCCACGCCTTCGGTATTGAACGCACA |
| 241 | GATACGGAAGTTGTACTCTGTGTTCTCTTTAAGCTTGGTCACTGTGAACTGCTTCCCTTGTAATCCCGATGGTGGCGTAC |
| 321 | AGGTAGTCCATTCGTCAGCCGCGGCTTCTTTGAGTTCAATCACATAGGCTCTAACGGGTGCGCCACCGTCGTAAATTGGC |
| 401 | TTATTCCATGCTAGGGAGACAGAAGATCT |

CARDIOTOX50

CARDIOTOX50 is a novel gene fragment. The nucleic acid was initially identified in a cloned fragment having the following sequence:

| (SEQ ID NO:55) | |
|---|---|
| 1 | CCATGGAAAATGGTGTTTGAGGCGAGGGGGTCGGTCACTGTGTCCAGTCCCATCACAAGACTGGGAAACATGCATGGGG |
| 81 | TTCGGGGTTTGGAAAAAGGAGGACAGAATTGATTAAAATTGAAATGGAGGATTATCTCTAAGATTTAGTCTCTGTAGAA |
| 161 | TTTTGTTTACAAATACTACCAAAAGGGTCATGATCGGGAGTGCTAGC |

The cloned sequence was assembled into a contig resulting in the following 1216 bp consensus sequence:

| (SEQ ID NO:56) | |
|---|---|
| 1 | TTTTTTTTTTTTTTTTTGAACTTTTGCCACTTTGTATTTTATTGTGGAACTCAGTTTCTTTTTTCTTTTTTTTTTTCCTT |
| 81 | TACATCAAATATCCTCAATAGAAGAGGGGATATTGCACACAAATACCATAAAAGCACTACATATTACTTTCACTGGAAAC |
| 161 | TAATTTTTCTACATTAGATATGACTGGATAGGATGGAAGTGATGCAGGATTATAAGACATAATACCATACACAGAGGCAG |
| 241 | ACCGACACAAACACCATTCAGAACAAGAGAGAGAGTGAGCTTCTCCACAGCCGGGCTTAGGACTGCACGCTGCCTGCGGG |
| 321 | CGCATGCGGGGAAAGCAAGGACCGCCGCGGCGTGGGCGGGCGGCTGAGCAGAGCCACTTCTCCGGGGCTCCAGTTTCGCG |
| 401 | AGCTCCACGCGTGCGGAGAAGCCGATTATTAGCTGTTGTTTTTTTTTCCCTTCCTTTTCAGTTTTTGATGCTGCCTTTGA |
| 481 | AATGAATTCTTAAAAGTTCCGGATTTTTGAAATAGTGAATAGTTTTAATACCAGGTGAATAAAACCTAATCGCTACCAAA |
| 561 | GCGCGGTGCTCATCCCTAGGCTGCTTTTGGTGTGTTGTTCAGCTGGTTACGTGATAAAAGCTTACAGTTCCTCTCACGTG |
| 641 | GAAACAGAATCTTTTTCTCCTAAATCTGAAGTATGAAAGGAAAAAAAAAGGAGAGAAGGAACGTCATTATCCTAACTCAC |
| 721 | AAATGTCATTGCCAAGCAGGGACCTCCTGTGACAAATGACAGAGGAGGTGAGAAAAAACAACTCCTGAATTGTAGTGCCG |
| 801 | CTCCAGGAGCTAAGATTTGTAACACAAATGGGAGGTGGTAAAATTTCCATTAGCAAATGATTAAATTTATAAAACGAGTA |
| 881 | TTAGAAAGCTCCTAAATTTCATAAGCTATTGGAAACACTTAAAACATTCATATACACCGGGGAAACCATTCACTATGATA |
| 961 | TGTAAGGTTAAGAAAAAAAATTTTTTTCTTTTGAATTCCATGGAAAATGGTGTTTGACGCGAGGGGGTCGGTCACTGTGT |
| 1041 | CCAGTCCCATCACAAGACTGGGAAAGCATGCATGGGGTTCGGGGTTTGGAAAAAAGGAGGACAGAATTGATTAAAATTGA |
| 1121 | AATGGAGGATTATCTCTAAGATTTAGTCTCTGTAGAATTTTGTTTACAAATACTACCAAAAGGGTCATGATCGGGAGTGC |
| 1201 | TAGCACAATAGAATTC |

CARDIOTOX51

CARDIOTOX51 is a novel gene fragment. The nucleic acid was initially identified in two cloned fragments having the following sequences:

| (SEQ ID NO:57) | |
|---|---|
| 1 | NAATTTGGTTTATTTCTCTATTCACTTGTTTTCAAGGCAAGAAAAATGTAGCTAAAGGAACAACTAGCCCTTTCTTCCAT |
| 81 | TTCTGTCTCCAAATTACTCACTAGT and: |
| (SEQ ID NO:58) | |
| 1 | TCATGACTGG0ASACTCTGATTCCTCCTCAGTCCACCCAATAAACTGCCACCAGAATTTAAATAGACAGCAGAGTCTGGT |
| 81 | TTTTGAAGACCCATTTCTGCCTCTCGGCTTTTCCCATTCTCCCGGGGAACAGGGGTCTTGACCACCCTGGCTATTCCCAG |
| 161 | CCTCTTCAGCCTGTCCACCAAGTTCATCTTCAGCTGGCCAACATCAGGAGGGGCCCTTGAAGGTCTCAAGCCATACATTT |
| 241 | CTTGCAGGAATGTTTCAGCTGGTCTGGAAGCCAAGAAATTC |

The cloned sequence was assembled into a contig resulting in the following 1115 bp consensus sequence:

| (SEQ ID NO:59) | |
|---|---|
| 1 | TTTTTTTTTTTTTTTTTGTGTTGTACAAAAATACAAGCTTAAAAAAAACTGAAGTTCTAATAATCACAAATACAAAGGGA |
| 81 | TCTATCTGGGTGGTGTTTGGGTTCTCCGTGCCCCAAAGTCCCCGGATAAGAAAGTCTCCATTTCTGATGTAAAGGACAAG |
| 161 | ATAAAATTCCTTATTTTGCTAACGCTGAGAGTGCACCATTGGATGGGTGCATTTGATCAGGGACCAGCAGGGAAGGCATC |
| 241 | TCCCACAGGCTCGGCTCACACCACTCTGCGCATGCACCAACTCTCCGGAACAGCCTCCTCCCAGCAACAGCCTGGGCTGC |
| 321 | CCCCGGTTTCCTTCGTAGGCAGGCGCTTCCAGCTTGTGTTCTCTAGAGACAAGGTGCCAGCACTTCGGTATTACTGTCAC |
| 401 | GTTTCGATAGAATTTGGTTTATTTCTCTATTCACTTGTTTTCAAGGCAAGAAAAATGTAGCTAAAGGAACAACTAGCCCT |
| 481 | TTCTTCCATTTCTGTCTCCAAATTACTCACTAGTCCCCACGTTACTAGACTCCATCCTCAAAAACCTTTGCGGCCGGCTC |
| 561 | TATCCCTCACTACGCCCTCTCCACATTCACAATCCTTCTACAACATCCCTTTTCTCTCAAGTTAGGCCGGTCCCAATTCT |
| 641 | CAGTGCATCTATCCTTCATGTGCTAATTTATTTACGAGGTCAGTTAATGTGGACCCCTCAGTCTTCCTTCAGGATACCCA |
| 721 | TTTTGGGCGAGGTTGTGCAAACTGGGGCTCCAAAGCTACCCATCATGACTGGGAGACTCTGATTCCTCCTCAGTCCACCC |
| 801 | AATAAACTGCCACCAGAATTTAAATAGACAGCAGAGTCTGGTTTTTGAAGACCCATTTCTGCCTCTCGGCTTTTCCCATT |
| 881 | CTCCCGGGGAACAGGGGTCTTGACCACCCTGGCTATTCCCAGCCTCTTCAGCCTGTCCACCAAGTTCATCTTCAGCTGGC |
| 961 | CAACATCAGGAGGGGCCCTTGAAGGTCTCAAGCCATACATTTCTTGCAGGAATGTTTCAGCTGGTCTGGAAGCCAAGAAA |
| 1041 | TTCTCGGAGACATGGACACGGGGTTCAAAGGGCACGGGGGAGGAACATGGTGACTGCGACGGAGGCGCAGGCAGC |

CARDIOTOX52

CARDIOTOX52 is a novel 153 bp gene fragment. The nucleic acid was initially identified in a cloned fragment having the following sequence:

| (SEQ ID NO:60) | |
|---|---|
| 1 | TGATCAATCTACTGTGAAAGACTCTCCTCCTGATACCTGTCCTCCTTCTGTAACGAAGCTTACTTAGCTTTTAGCTGTGA |
| 81 | AAAACTCTGGGAACTTCCCCACCCATTAATTCTTATAAAGTCAAGTCCCCAAACTGGATGTGTCTCAGTGCAC |

CARDIOTOX53

CARDIOTOX53 is a novel 89 bp gene fragment. The nucleic acid was initially identified in a cloned fragment having the following sequence:

| (SEQ ID NO:61) | |
|---|---|
| 1 | AGATCTGCAGCATGACCGGGCCCGTCTCTGGGTCGTTCATCCACTGGGTGCTGTTAAGTGGGTTCTCCAGCATGTCTTCA |
| 81 | AATGCTAGC |

CARDIOTOX58

CARDIOTOX58 is a novel gene fragment. The nucleic acid was initially identified in a cloned fragment having the following sequence:

| (SEQ ID NO:62) | |
|---|---|
| 1 | CCTAGGAAGCGGAGGTTTAGAATCTTGATCTGCTGGTCTTCCAGGTCCATTCGGATGATGCCATCCTCACCATCAATACT |
| 81 | CAGAAGGACCCCGGTAGCCTCTCGGTCCTCACCCAGAATCACTTTCACCTTGTTGTTCTTGGTGGGGGTGATGGGCTCCA |
| 161 | GATGCTCACTGGAGATACTGACCACCTTCTCACTATCTTTCAGGTACACGGAGCACATGCCTCCCGTGACACTGCGGATG |
| 241 | ACGCCTGTCTGCCCCACTATTTGTGTGTCCAGATAGGTGTCTCGAACCTTCACCTGGATATCAGTGGTCACCCAGTCACT |
| 321 | GGAGTTCTGCTCAATGCCTGAGCCTGGTGTGTGGGGATTGTAGCCTCCAGGAGAAGGAGCTCCAGGGGTCATTGGACTGT |
| 401 | AGCCAACAGGGCTGGGGCTGGGACTAGCCTGATAGGCCATGG |

The cloned sequence was assembled into a contig resulting in the following 710 bp consensus sequence:

| (SEQ ID NO:63) | |
|---|---|
| 1 | TTTTTTTTTTTTTTTTTTTTTTTTCAAACAGTTTCTCTTTATTGAAAGGCCTGAACACAAAGGCAAGCTGGGACAGCAGA |
| 81 | AAGAAGGCAGGACATTCCTCAGACTGCTCTGATTCCTAGAGTACCAGGGGAGGAGGAAAAGGAAATCCAGAGTGATTGCC |
| 161 | CTGGCTTGCCCCAGACTCGGGGTTCCATCCTAGGCCAAGCAAGGCCAAAGCGGGCTGCTTGCTCCGTGTCTGCACTGCAC |
| 241 | GCTTGGGCCTCAGGCCTCCAGGAGCTTCCCTAGGAAGCGGAGGTTTAGAATCTTGATCTGCTGGTCTTCCAGGTCCATTC |
| 321 | GGATGATGCCATCCTCACCATCAATACTCAGAAGGACCCCGGTAGCCTCTCGGTCCTCACCCAGAATCACTTTCACCTTG |
| 401 | TTGTTCTTGGTGGGGGTGATGGGCTCCAGATGCTCACTGGAGATACTGACCACCTTCTCACTATCTTTCAGGTACACGGA |
| 481 | GCACATGCCTCCCGTGACACTGCGGATGACGCCTGTCTGCCCCACTATTTGTGTGTCCAGATAGGTGTCTCGAACCTTCA |
| 561 | CCTGGATATCAGTGGTCACCCAGTCACTGGAGTTCTGCTCAATGCCTGAGCCTGGTGTGTGGGGATTGTAGCCTCCAGGA |
| 641 | GAAGGAGCTCCAGGGGTCATTGGACTGTAGCCAACAGGGCTGGGGCTGGGACTAGCCTGATAGGCCATGG |

CARDIOTOX59

CARDIOTOX59 is a novel gene fragment. The nucleic acid was initially identified in a cloned fragment having the following sequence:

| (SEQ ID NO:64) | |
|---|---|
| 1 | GCTTATGGTAAGGAGGCTCCATTTCTCTTGTCCTTTCGTACTGGGAGAAATTGTAAATAGATAGAAACCGACCTGGATTG |
| 81 | CTCCGGTCTGAACTCAGATCACGTAGGACTTTAATCGTTGAACAAACGAACCATTAATAGCTTCTGCACCATTGGGATGT |
| 161 | CCTGATCCAACATCGAGGTCGTAAACCCTAATTGTCGATATGAACTCTTAAATAGGATTGCGCTGTTATCCCTAGG |

The cloned sequence was assembled into a contig resulting in the following 1618 bp consensus sequence:

| (SEQ ID NO:65) | |
|---|---|
| 1 | TCCATTTTGTTCCTTCCTCCGGTTGTGCCCCCCGGTTCCTCTTTTTCTTTTTTAACCTGGGCTAGGTTTATTTATTGTAC |
| 81 | ATATATACTTTATTGAGATTTTTTTCATAAATTGGTTGGGAGCACTTATGGTAAGGAGGCTCCATTTCTCTTGTCCTTTC |
| 161 | GTACTGGGAGAAATTGTAAATAGATAGAAACCGACCTGGATTGCTCCGGTCTGAACTCAGATCACGTAGGACTTTAATCG |
| 241 | TTGAACAAACGAACCATTAATAGCTTCTGCACCATTGGGATGTCCTGATCCAACATCGAGGTCGTAAACCCTAATTGTCG |
| 321 | ATATGAACTCTTAAATAGGATTGCGCTGTTATCCCTAGGGTAACTTGGTCCGTTGATCAATAATTGGGTCAATAAGATAT |
| 401 | TAGTATTACTTTGACTTGTGAGTCTAGGTTAAAATCATTCGGAGGATTTTTTATTCTCCGAGGTCACCCCAACCGAAATT |
| 481 | TTTTAGTTCATATTTATTTTGTTTTAGCCCATTAGGTTGTTTTTATATAAGTTGAACTAGTAAATTGAAGCTCCATAGGG |
| 561 | TCTTCTCGTCTTATTGGGAGATTCCAGCCTCTTCACTGGAAGGTCAATTTCACTGATTGAAAGTAAGAGACAGTTGAACC |
| 641 | CTCGTTTAGCCATTCATTCTAGTCCCTAATTAAGGAACAAGTGATTATGCTACCTTTGCACGGTCAGGATACCCCGGCCG |
| 721 | TTTAACTTTAGTCACTGGGCAGGCAATGCCTCTAATACTTGTTATGCTAGAGGTGATGTTTTTGGTAAACAGGCGGGGTT |
| 801 | CGTGTTTGCCGAGTTCCTTTTACTTTTTTTAATCTTTCCTTAAAGCACGCCTGTGTTCGGCTAACGAGTTAGGGATAGGT |
| 881 | AATTTTATTGTTGGGTTAGTACCTATGATTCGATAATTGACAATGGTTATCCGGGTTGTCATACACTTGTGCTAGGAGAA |
| 961 | TTGGTTCTTGTTACTCATATTAACAGTATTTCATCTATGGGTCTATAGATTAGCCCAATTTGTAATATAGGAATTTATTG |
| 1041 | AGGTTTGTGGAATTAGTGTGTGTAAGTATGTATGTTGAGCTTGAACGCTTTCTTTATTGATGGCTGCTTTTAAGCCTACA |
| 1121 | ATGGTTAAGTGGTTGTAGTTGTTTATTCACTATTTAAGGTTTTTTCCTTTTCCTAAAGAGCTGTCCCTCTTTTGGTTATA |
| 1201 | TTTTAAGTTTACATTTTGATTTGTTGTTCTGATGGTAAGCTTAAAGTTGAACTGAAATTCTTTTTTGGGCAACCAGCTAT |
| 1281 | CACCAAGCTCGATAGGCTTTTCACCTCTACCTAAAAATCTTCCCACTATTTTGCTACATAGACGGGTTGATTCATGAAAT |
| 1361 | TGTTTTTAGGTAGCTCGTTTGGTTTCGGGGTTCTTAGCTTAAATTCTTTTTGTTAAGGATTTTCTAGTTAATTCATTATG |
| 1441 | CAAAAGGTACAAGGTTTAATCTTTGCTTATTTTTACTTTAAATTAGTCTTTCACCATTCCCTTGCGGTACTTTCTCTATA |
| 1521 | GCTCCTGGTAAGTAAATTTCTTTCTCCAATACTTTTTGAGTTAAATGTTTTAGTTTATGTGGGGGGGGGTTAGTTATGTT |
| 1601 | GGTTGGTTGCCTCGTGCC |

CARDIOTOX60

CARDIOTOX60 is a novel gene fragment. The nucleic acid was initially identified in a cloned fragment having the following sequence:

| | | |
|---|---|---|
| 1 | TGTACAGGCTGTATTCCTCATGCCCAATGGCACGCTGTCTGCCCCGAGTGGAGATCT | (SEQ ID NO:66) |

The cloned sequence was assembled into a contig resulting in the following 186 bp consensus sequence:

| (SEQ ID NO:67) | |
|---|---|
| 1 | NAATCTCTTTGTTGCCTAGACCTGTGCCCCTGCCACAGAGCCTCGCAGGGACTGGTCACCTGCCGTGTGCTGGCTGCTGC |
| 81 | TGAGTCACTCTTCTGGAAGCTGGGGCAGAGGTGGCCAAGATGTCGACTGAGATCTCCACTCGGGGCAGACAGCGTGCCAT |
| 161 | TGGGCATGAGGAATACAGCCTGTACA |

CARDIOTOX61

CARDIOTOX61 is a novel 238 bp gene fragment. The nucleic acid was initially identified in a cloned fragment having the following sequence:

| (SEQ ID NO:68) | |
|---|---|
| 1 | GAATTCGCCCTAAAGATGCTGCAGGACTGTCCCAAGGCACGCAGAGAGGTGGAGCTACACTGGAGGGCCTCCCAGTGCCC |
| 81 | ACACATCGTGCACATCGTGGACGTCTATGAGAACCTGTATGCCGGGAGGAAGTGCTTGCTGATTGTCATGGAGTGTCTCG |
| 161 | ATGGTGGAGAGCTCTTTAGTCGGATCCAGGACCGAGGAGACCAGGCATTCACAGAAAGAGAGGCATCAGAGATCATGA |

CARDIOTOX62

CARDIOTOX62 is a novel 173 bp gene fragment. The nucleic acid was initially identified in a cloned fragment having the following sequence:

| (SEQ ID NO:69) | |
|---|---|
| 1 | CCATGGTGGGGCCTCACGGCTACATCTCTGCATCTGACTGGCCTCTCATGATTTTTTACATGGTGATGTGTATTGTTTAC |
| 81 | ATATTATATGGTGTCCTCTGGCTGCTGTGGTCTGCCTGTTACTGGAAAGATATACTGAGAATCCAGTTCTGGATTGCAGC |
| 161 | TGTTATTTTCCTAGG |

CARDIOTOX63

CARDIOTOX63 is a novel 133 bp gene fragment. The nucleic acid was initially identified in a cloned fragment having the following sequence:

| (SEQ ID NO:70) | |
|---|---|
| 1 | GTGCACTCGAATTCCAGGTCCTACCTGTGGCAGGAAGAGCCCATGATGGGAGCTTGAATCTACCCCCATTCCTACTGGGC |
| 81 | CCAGAGCTCCCCTCTGACCAGCAGAGATAGCCCCTGCCAGCCCCAGCTAGC |

CARDIOTOX64

CARDIOTOX64 is a novel gene fragment. The nucleic acid was initially identified in a cloned fragment having the following sequence:

| (SEQ ID NO:71) | |
|---|---|
| 1 | TCCGGAAGAGCAATCAGTGCTCTTAACCGCTGAGCCACCTCTCCAGCCCTGAAGGGCTCTTTCAAAGGTTTATTCTTTCT |
| 81 | CCTTTCACAAGTCGGCATCGAAACTTCCAAGTGTCCTCAAAGTCCAGGGCTCCTTGGACTCCATAACGTTTCTCCGCAAT |
| 161 | CTCAATAACTTCCCTCGCAATGTTTTCTTGACTGGTGCCCTTCACGCTGATATATTTGCAGTCGGAGCTGCCATAGTGGC |
| 241 | AGGAGATTGCCTGCGCAGAAAGGACCGGCCGGAGAAGGGCAGTTTATCAATCCCATTGTGCCCCGAAACCAAGCAGAGCC |
| 321 | CTCCGAAGAGGAATGCTTCACTTGGGATTTGATTTCTCAATTG |

The cloned sequence was assembled into a contig resulting in the following 477 bp consensus sequence:

| (SEQ ID NO:72) | |
|---|---|
| 1 | ATTATTTATATGAGTACACTGTAGCTATCTTCAGACACACCAGAAGAGGGCACCAGATCCCATTACAGATGGTTGTGAGC |
| 81 | CATCATGTGGTTGCTGGGATTTGAACTCAGGACCTCCGGAAGAGCAATCAGTGCTCTTAACCGCTGAGCCACCTCTCCAG |
| 161 | CCCTGAAGGGCTCTTTCAAAGGTTTATTCTTTCTCCTTTCACAAGTCGGCATCGAAACTTCCAAGTGTCCTCAAAGTCCA |
| 241 | GGGCTCCTTGGACTCCATAACGTTTCTCCGCAATCTCAATAACTTCCCTCGCAATGTTTTCTTGACTGGTGCCCTTCACG |
| 321 | CTGATATATTTGCAGTCGGAGCTGCCATAGTGGCAGGAGATTGCCTGCGCAGAAAGGACCGGCCGGAGAAGGGCAGTTTA |
| 401 | TCAATCCCATTGTGCCCCGAAACCAAGCAGAGCCCTCCGAAGAGGAATGCTTCACTTGGGATTTGATTTCTCAATTG |

CARDIOTOX65

CARDIOTOX65 is a novel 413 bp gene fragment. The nucleic acid was initially identified in a cloned fragment having the following sequence:

| (SEQ ID NO:73) | |
|---|---|
| 1 | CAATTGATGCTGATGTGACAGTGATAGGTTCTGGTCCTGGAGGATATGTTGCTGCCATCAAAGCTGCCCAGTTAGGCTTT |
| 81 | AAGACAGTCTGCATTGAGAAGAATGAAACACTAGGAGGAACATGCTTGAATGTTGGTTGTATTCCTTCAAAGGCTTTATT |
| 161 | AAATAATTCTCATTATTACCATTTGGCCCATGGAAAAGATTTTGCATCTAGGGGAATTGAAATACCAGAAGTTCGCTTGA |
| 241 | ATTTAGAGAAGATGATGGAGCAGAAGCGTTCTGCAGTAAAAGCATTAACAGGGGGAATTGCCCACTTATTCAAACAAAAT |
| 321 | AAGGTTGTTCATGTCAATGGATTTGGAAAGATAACTGGCAAGAATCAGGTTACAGCTACAACGGCCGATGGCAGCACTCA |
| 401 | GGTTATTGGTACC |

CARDIOTOX66

CARDIOTOX66 is a novel gene fragment. The nucleic acid was initially identified in a cloned fragment having the following sequence:

| (SEQ ID NO:74) | |
|---|---|
| 1 | TGATCATAATCTGTGAAGTGACTCCTTGTTCATGAGAGCAGATTTTTAACAAGACGAGTATGAGAGGAAACCTAGGTAAG |
| 81 | CTATGATGTATAATCACATAAGCTGGTCCTGTAGCTGTCAGGTTTTTCAGTAGGAACGGATAGCAGGAGGTACC |

The cloned sequence was assembled into a contig resulting in the following 726 bp consensus sequence:

| (SEQ ID NO:75) | |
|---|---|
| 1 | TTTTTTTTTTTTTTTTTCAAATACTATGTGGTTTTATTTAGAAATAAAAGATTGTTCGTTTGTACATCATTTAAGAATTA |
| 81 | TACCAGTTTATCACTGCACAAGAATGAGAGACAATGGTGCAACTCGAGTCCCTCACATCAAGTACTGCCAAGCTGTTGAT |
| 161 | CATAATCTGTGAAGTGACTCCTTGTTCATGAGAGCAGATTTTTAACAAGACGAGTATGAGAGGAAACCTAGGTAAGCTAT |
| 241 | GATGTATAATCACATAAGCTGGTCCTGTAGCTGTCAGGTTTTTCAGTAGGAACGGATAGCAGGAGGTACAGTAGCACAGT |
| 321 | CAGCCTCATTCAAGGTCTTGTCAATAACAGGTCTGTAATCCAAAGTAACCTTCCCAGTCTTGGTGTCCACATATGAGAGG |
| 401 | CTGTGCTTCCTCCAGTGTTCCGCAAATGGCTTCTTCTGCTGGCCCTCGATGGGCTTGGAGTAATCATACTCATCAATCCG |
| 481 | CACCTTGTAATCTTCCCTGGCATGAGCTCCCCGTGACTCCTTCCGTGCTTCCGCACCATATATGGTCTGCAGTGCGCACA |
| 561 | GCATCAGATTCTGCAGCTCCAGCGTCTCCACCAGGTCTGTGTTCCAGACCATTCCCCTGTCAAACGTCTTCAGATGCTGT |
| 641 | AGGTCTCCATAGAGCTGGCTGACTTTTTCACAGCCTTCTTGCAGCACACTTCCCACACGGGACACGGCGGCATGGCTCTG |
| 721 | CATCGA |

CARDIOTOX67

CARDIOTOX67 is a novel gene fragment. The nucleic acid was initially identified in a cloned fragment having the following sequence:

| (SEQ ID NO:76) | |
|---|---|
| 1 | TGTACGGTCATTTCTTCTGCCTTCCGTCTCTGCGACTCTCGGAGAACTTCCAGCAGCAGCATGTTGGGCCAGAGTATCCG |
| 81 | GA |

The cloned sequence was assembled into a contig resulting in the following 440 bp consensus sequence:

| (SEQ ID NO:77) | |
|---|---|
| 1 | TTTTTTTTTTTTTTTTTATTTATATCATTAGTTTATTTACATTTTTTTCTAGTATAAGAGTTCAAGAGTTTAATCCAATT |
| 81 | TCCAGATCATATCTCTTAAACTTTCTTCATTCTGTTAATGGGATGAATTAAATATCCTTATTTTTTAAGTAGCTGGTGCC |
| 161 | TTACTATAAAGAAAGGAGCAGCAAATCCAGATCCAAAGTACACGGTCATCATAAGCAATAACCGCCACTTGTTTTCCACT |
| 241 | GAAAACGGCAAATTCTTCCCCGGACCCTCCTCATAGTGGCTGCGACGCACCACGGAGGTGGTGAACCTCCGGATACTCTG |
| 321 | GCCCAACATGCTGCTGCTGCAAGTTCTCCGAGAGTCGCAGAGACGGAAGGCAGAAGAAATGACCGTACCACCTCACCCTA |
| 401 | CTTTCTTCACGACCTTGCTATCCGGAACGAGCCTCGTGCC |

CARDIOTOX68

CARDIOTOX68 is a novel 276 bp gene fragment. The nucleic acid was initially identified in a cloned fragment having the following sequence:

| (SEQ ID NO:78) | |
|---|---|
| 1 | GGTACCATCTCCTGGCCATCCCCTCGATTAACCAAGCTATTCATGTATTCTTATGCCAGAGCAGTGTCAACTCCTGGAGG |
| 81 | TCCCGGGTGCAGCAGATGCCTCGTGTGGTAGTTCTAAATTTAAATTTCACTGGAAACTGGGCAACCAAGCAATGAGCCAC |
| 161 | AGCAAAATAAGAGAAGCATCACCACCAATGAACCTGTTGTTAAAACCATACTACCAACTGCCCATAAAAAATTACTGATT |
| 241 | TGATGTATTCTTTTTCATGTCAGCATATGTTCAATTG |

CARDIOTOX69

CARDIOTOX69 is a novel 149 bp gene fragment. The nucleic acid was initially identified in a cloned fragment having the following sequence:

| (SEQ ID NO:79) | |
|---|---|
| 1 | GGTACCACTGTTTTCCTAGTTTCCTTTGTTATCTGTCCATGAGTGAGGTGCGTTTGATCCTGTTGTATGGCAGTTTCCTC |
| 81 | TTGAATTCCCACAGCTGCCTCTAGCTTTGTGGACTTGGCGGTGGCAACCACCACGGATGCAGCAATTG |

CARDIOTOX70

CARDIOTOX70 is a novel gene fragment. The nucleic acid was initially identified in a cloned fragment having the following sequence:

| (SEQ ID NO:80) | |
|---|---|
| 1 | AGATCTGGAGAATTGAAGGTTCCAACAAGGTACTGGTGGACCCCGCCACATACGGCCAGTTCTATGCAGGTGACAGCTAC |
| 81 | ATCATTCTGTACAACTACCGCCATGGTGGCCGCCAGGGACAGATCATCTACAACTGGCAGGGTGCCCAGTCTACCCAGGA |
| 161 | TGAGGTCGCTGCTTCAGCCATCCTGACTGCCCAGCTGGATGAGGAACTGGGAGGAACTCCTGTCCAGAGCCGAGTGGTCC |
| 241 | AAGGCAAAGAGCCTGCACACCTCATGAGCTTGTTTGGTGGGAAGCCCATGATCATCTACAAGGGTGGCACCTCCCGAGAT |
| 321 | GGTGGGCAGACAACCCCTGCCAGTACC |

The cloned sequence was assembled into a contig resulting in the following 467 bp consensus sequence:

| (SEQ ID NO:81) | |
|---|---|
| 1 | AGTACTGGCAGGGGTTGTCTGCCCACCATCTCGGGAGGTGCCACCCTTGTAGATGATCATGGGCTTCCCACCAAACAAGC |
| 81 | TCATGAGGTGTGCAGGCTCTTTGCCTTGGACCACTCGGCTCTGGACAGGAGTTCCTCCCAGTTCCTCATCCAGCTGGGCA |
| 161 | GTCAGGATGGCTGAAGCAGCGACCTCATCCTGGGTAGACTGGGCACCCTGCCAGTTGTAGATGATCTGTCCCTGGCGGCC |
| 241 | ACCATGGCGGTAGTTGTACAGAATGATGTAGCTGTCACCTCCATAGAACTGGCCGTATGTGGCGGGGTCCACCAGTACCT |
| 321 | TGTTGGAACCTTCAATTCTCCAGATCTGTTTCTGGCCAGTTCCGTCATCATCCATGCCGTGCTGGGCAGCCATGGCGGTG |
| 401 | GAGGTGTGCAGTGTAGCAGCATCGAAAGGCACGCGCTCCACGTTGGCAATGTGGCTGGAGAGGTACC |

CARDIOTOX71

CARDIOTOX71 is a novel gene fragment. The nucleic acid was initially identified in a cloned fragment having the following sequence:

| (SEQ ID NO:82) | |
|---|---|
| 1 | TCATGAGGGCGTGGAGTAGACACTGGCTTTGCACAGAGTTGCCCATGCCTGTTCTCCTAATCCAACTGGACCCCGTGGT |
| 81 | AGGAGTGCACCCGGC |

The cloned sequence was assembled into a contig resulting in the following 535 bp consensus sequence:

| (SEQ ID NO:83) | |
|---|---|
| 1 | TTTTTTTTTTTTTTTTTTCCAAGGAGAGAGGATTTATTTGTGTTCCCTGGGACGGGAACAGGGAGAGTCCAGAAGAGCCA |
| 81 | AAGTTTCAAGGACACAACCAGGTTCAGAGAGTCTAGAGAACCCGGGTGCACTCCTACCACGGGGTCCAGTTGGATTAGGA |
| 161 | GAACAGGCATGGGCAACTCTGTGCAAAGCCAGTGTCTACTCCACGCCCTTCATGAACTCCAGGAACTCGTCATAGTCGAT |
| 241 | TCGGCCATCGTTGTTCTTGTCACCGTCCTTCATGAGCTCTTCGATGTCATCTTCCGTGATGGTCTCACCTGTGGCCTGCA |
| 321 | GCATCATCTTCAGTTCATCCAAGTCAATGTAGCCATCAGCGTTTTTGTCAAACATGCGGAAGAGATCCGACAGCTCCTCC |
| 401 | TCAGACTTCCCTTTGCTGTCATCCTTCATGCACCGAACCATCATGACAAGGAACTCGTCGAAGTCCACTGTGCCACTGCC |
| 481 | ATCCTCATCTACCTCGTCGATCATCTCCTGCAGCTCCTCAGGTGTGGGGTTCTGT |

CARDIOTOX72

CARDIOTOX72 is a novel gene fragment. The nucleic acid was initially identified in a cloned fragment having the following sequence:

| (SEQ ID NO:84) | |
|---|---|
| 1 | GCCGGGGACACTGCCTGGGCCTGAGTATGGGGGCATTCTCTTGATGCAGTACTGGGCCTGATCCGGAGGCAGCTCTCGAC |
| 81 | GAAGTTCCTCTGCCAAGATGTAAGGCTTATCAGAAGCCAGAATCCGGAAGGAGGCGATGACCTGTTCTGCAGTGTCCGTG |
| 161 | TCTGCGGTCTCTCTAGTCATGA |

The cloned sequence was assembled into a contig resulting in the following 445 bp consensus sequence:

| (SEQ ID NO:85) | |
|---|---|
| 1 | TTTTTTTTTTTTTTTTTCCAGGTAACAACCTACACTTGAGCCTTTATTGCGTTCTGATAGGGTCAGGGGTTACAGAAGGA |
| 81 | GCATCAGAGGTCGCTCTCCCCGTAGAGGGCAGAGGAGAAGGCAGTGTAGTCCAGGGCCCCGGGGACACTGCCTGGGCCTG |
| 161 | AGTATGGGGGCATTCTCTTGATGCAGTACTGGGCCTGATCCGGAGGCAGCTCTCGACGAAGTTCCTCTGCCAAGATGTAA |
| 241 | GGCTTATCAGAAGCCAGAATCCGGAAGGAGGCGATGACCTGTTCTGCAGTGTCCGTGTCTGCGGTCTCTCTAGTCATGAA |
| 321 | GTCAATGAAGGACTGGAAGGTGACTGTGCCTTGTCCCTTGGGGTCAACCAGACTCATAATTCGGGGAAACTCAGCTTCAC |
| 401 | CCAAGTCATACCCCATGGAAATGAGGCACCCCCTCGTGCCGAATT |

CARDIOTOX73

CARDIOTOX73 is a novel 246 hp gene fragment. The nucleic acid was initially identified in a cloned fragment having the following sequence:

(SEQ ID NO:85)

TCCGGAGTGGGATGCCCACTTCATCCATAGACACACTGCTTAGGTCCTGT
GCACTCCTCACCACCCGTCTGCTGTCATCCTTGGCTCTCCTTTCCGCAGC
CCTGATGGGCGAGGTGAGTTCTGCCGGGGTTGGCACTGGGTCCTGCTCAC
CCACTCTTCTCTCTGAGGCGGGATCTGAAAGACTACTGAGTCGTTTTTGC
TGTTCTCGGTTGTGCTGCAAGAGCACAATGGTAGGGTTGACAATTG

CARDIOTOX74

CARDIOTOX74 is a novel 126 bp gene fragment. The nucleic acid was initially identified in a cloned fragment having the following sequence:

| (SEQ ID NO:87) | |
|---|---|
| 1 | CAATTGTATTCTTGCTGACTAAGGTTCAAGGAGACTGGTTTTTCTGAGAAGCCATCCCTGGTAAATTGACAGTAGTTCAG |
| 81 | AGAGTTTAGTCTTATCTTGTCATGAGCTGGTAACCACTGGGGTACC |

CARDIOTOX75

CARDIOTOX75 is a novel gene fragment. The nucleic acid was initially identified in a cloned fragment having the following sequence:

| (SEQ ID NO:88) | |
|---|---|
| 1 | TCCGGAAATGTGGGAGCTGAGCGCCCGGCAGACACGCTGCTATGCAGGGGCTATTTGGGGCTTGCTTTTAGGGATTTGTT |
| 81 | TCCAATTG |

The cloned sequence was assembled into a contig resulting in the following 370 bp consensus sequence:

| (SEQ ID NO:89) | |
|---|---|
| 1 | TGTACAGGAGGTGAGCAAAGGCAGGGGAGAGGAGAGGTTCTGGAGCGGGGTTGGCATGAGCTGGGAGCTCCACAATAGCC |
| 81 | GTGGCCCTCTGAGAAAGAAGGGTAGTGTTTGTGAGGCCAGATGCTGCTCTCTTGGCTCTCTGACTGACTGGACATGCTGC |
| 161 | TGGCCATTTGGCTATCTGCCTCTTCAGCTATGGACTTTATTTATGGGAAGATTAAACAAGGTGAGAAAGCTCAATTGGAA |
| 241 | ACAAATCCCTAAAAGCAAGCCCCAAATAGCCCCTGCATAGCAGCGTGTCTGCCGGGCGCTCAGCTCCCACATTTCCGGAG |
| 321 | TAGCATGAAACTTGTCAGCCCTTATCCTAGGCCCTGGGATGTTAAAGCTT |

CARDIOTOX76

CARDIOTOX76 is a novel 337 bp gene fragment. The nucleic acid was initially identified in a cloned fragment having the following sequence:

| (SEQ ID NO:90) | |
|---|---|
| 1 | AAGCTCGAGGGTGGAATCAAGGTACCAGAATGTGGATATTTCTTCACCCGGGGTGAATGTGGAAGCTCCTGATATTCACG |
| 81 | TGAAAGCTCCCAAGTTCAAGGTGCCAGGCGTGGAAGCCGCAGGGCCAAAAATAGAGGGCAACTTGAAAGGTCCCAAGGTG |
| 161 | CAGGCAAACCTGGACACACCAGACATCAATATCCAAGGTCCGGAAGCTAAAATCAAAACCCCCTCTTTTAGTGTGTCGGC |
| 241 | TCCTCAAGTCTCCATACCCGATGTGAATGTTAAATTGAAAGGACCAAACATAAAGGGTGATGTTCCCAGTGTGGGACTGG |
| 321 | AGGGACCTGACGTAGATCT |

CARDIOTOX77

CARDIOTOX77 is a novel 100 bp gene fragment. The nucleic acid was initially identified in a cloned fragment having the following sequence:

| (SEQ ID NO:91) | |
|---|---|
| 1 | CCATGGGCACAGGCTGCGCCCGAGGCTTCCTGGCAGCCTTTGACACGGCATGGATGGTAAAGAGCTGGGACCAGGGCACC |
| 81 | CCTCCCCTGGAGGTGCTAGC |

CARDIOTOX78

CARDIOTOX78 is a novel 44 bp gene fragment. The nucleic acid was initially identified in a cloned fragment having the following sequence:

(SEQ ID NO:92)

| | |
|---|---|
| 1 | GCTAGCATGACACCAACAAGGACCCTATCTTGAGGAAAAGATCT |

CARDIOTOX79

CARDIOTOX79 is a novel gene fragment. The nucleic acid was initially identified in a cloned fragment having the following sequence:

| (SEQ ID NO:93) | |
|---|---|
| 1 | CCTAGGACTGTGGGGACACTTGGGCCTTCCGCATGGATCGAAGGGCCTTCTCCCGAAGGTGCCTCTCTAAGTCATCAAGG |
| 81 | TTGTCATCTTCAGCTTCACTCTCAGTCTCCTTCCTGGGCTCTGGTGCTGCCGCAGGCTCTTCCTGGGCTGATGGAGTGGC |
| 161 | GGCAGCAGAGACAGCTGCAGGGGCGGCAGGAGCTGGGGTGGCTACGGCCACAGCCTTCTCCTTCTTGTGTTTTTTGTGCT |
| 241 | TCTTCTGTTTCTTATCCTTCTTATGTTTCTTGTCCTTCTTCTTCTTCTTCTTCTTTCCACCTCCTTCTTGATCA |

The cloned sequence was assembled into a contig resulting in the following 698 bp consensus sequence:

| (SEQ ID NO:94) | |
|---|---|
| 1 | CCTAGGACTGTGGGGACACTTGGGCCTTCCGCATGGATCGAAGGGCCTTCTCCCGAAGGTGCCTCTCTAAGTCATCAAGG |
| 81 | TTGTCATCTTCAGCTTCACTCTCAGTCTCCTTCCTGGGCTCTGGTGCTGCCGCAGGCTCTTCCTGGGCTGATGGAGTGGC |
| 161 | GGCAGCAGAGACAGCTGCAGGGGCGGCAGGAGCTGGGGTGGCTACGGCCACAGCCTTCTCCTTCTTGTGTTTTTTGTGCT |
| 321 | CTGGCAGGGGACGGGCTTGGTGTTGGGCTTTTAGCCTTCTTGGCTGGTGCTGCAGGTGACCAGTTTGTGGAGGGTGACTG |
| 401 | AGACTGCACAACAGAGGGGGGTGCTGGAGGCTTTTTAGCTGTTGGCTCAGGAGATCCAGAGACAGAGCGGGAAGATGAAA |
| 481 | CCCTTCTTACOGACTGAGGGCTTGGTGAGGCAGCCTTTTTTATCTTTTTGGGTTCCGGAGTCCTGGAGACTCTCCTAATA |
| 561 | GGCCTAGTACTCGGAGACGGGGACTGCCTTCCTTGGGGAGACGCTGAAGCTCCTCTTCGAACAGGGGGAGGGCTTGAGGT |
| 641 | CTGAGGCGCCCGAGGTCGTGGTGAGGGCGAGTGCCTTTTGTTTGGTTGTGGTGACCGG |

CARDIOTOX80

CARDIOTOX80 is a novel gene fragment. The nucleic acid was initially identified in a cloned fragment having the following sequence:

| (SEQ ID NO:95) | |
|---|---|
| 1 | GGTACTTTTAAGATAAAGTCTAGTCCAGTTTAAATGTCAACTAGTGCAAAAGCTAGTGACAAAGCTGGATACCAAAAATA |
| 81 | GCCAACACTACAACATAAACACTTTGTATTCAAAGTATACAATTCACTTTATAAATTATTAATGGTATATAATTTGTATA |
| 161 | AAATATATTGCTGCTGTCCAGCATGCTTTTTTTTAAAATCCAAACACAAGGCCAGGAGGATAGTTAATTTGAAGAATAGA |
| 241 | TAACTTCCATTACACTACACATTTAACAATGCTTAAATGTTTGTTTACTGCCATGCAATTG |

The cloned sequence was assembled into a contig resulting in the following 660 bp consensus sequence:

| (SEQ ID NO:96) | |
|---|---|
| 1 | TTTTTTTTTTTTTTTTTTCTTTGAATTCGTTTATTTAAGAGATAGAACACAGCCATTCAAACTTGTGAAACAAAGTATTA |
| 81 | ACACGGGATAAGGTTGGAAAATTAAGATGAATTGCTCTATTCCATTTGCACAATAAATATTTTTAAAGAAGCTTGTAGAT |
| 161 | CTTTAAAAGCTTTTAAACTAGATACTAACATAAATAAGCATTTCTATCTAAATTGAGGCATACTGATTTTCAATAGAATT |
| 241 | ATAATATCAATTGCATGGCAGTAAACAAACATTTAAGCATTGTTAAATGTGTAGTGTAATGGAAGTTATCTATTCTTCAA |
| 321 | ATTAACTATCCTCCTGGCCTTGTGTTTGGATTTTAAAAAAAAGCATGCTGGACAGCAGCAATATATTTTATACAAATTAT |
| 401 | ATACCATTAATAATTTATAAAGTGAATTGTATACTTTGAATACAAAGTGTTTATGTTGTAGTGTTGGCTATTTTTGGTAT |
| 481 | CCAGCTTTGTCACTAGCTTTTGCACTAGTTGACATTTAAACTGGACTAGACTTTATCTTAAAAGTACCTAACCCGAGCCT |
| 561 | AATATTTTATGTCCTCTAAGGTTTCCCATTTTGTTTGGGAGACGTAGTTTGAAATTTTTCTAACATAATATCCTTTTCAA |
| 641 | AATTGTGTCTACATGAAGAG |

CARDIOTOX81

CARDIOTOX81 is a novel 115 bp gene fragment. The nucleic acid was initially identified in a cloned fragment having the following sequence:

| (SEQ ID NO:97) | |
|---|---|
| 1 | GGATCCAAAATAAAATCAAGTTCCTAATGGTGGGAGOTGTCAATCCTCTTGTGAGAAAAAGATTGATTGTATAGCTTATA |
| 81 | AAATTTGCAAGACAGGTTTAAAGGAGTAAGCTT |

CARDIOTOX82

CARDIOTOX82 is a novel 294 bp gene fragment. The nucleic acid was initially identified in a cloned fragment having the following sequence:

| (SEQ ID NO:98) | |
|---|---|
| 1 | GCCGGGGGTCCAGAAGGGAGAGTCCCAGACTCGCTACTCTGCGACAGGGTGCGGGATCGGGACCGACTGCCATCGATGGA |
| 81 | TGCCGCACTGGTCAGAGATGCTGTGCGAGACCGAGACAGGCGAGTCATACAGGATGAGGCCATGTAGCCCATGCCTTGCA |
| 161 | CGAAGTACTTGAAAGCTTCTGTCAGCTTGCCTGGCTGAGTCAGCTGCGGCTGACCTCCAGAGTCCGCCATCTTGAGGAAT |
| 241 | GAGGTCTGTGTGGCGTCCAGTTTTGAATTACATTCCACCACGGCATCTTCATGA |

CARDIOTOX83

CARDIOTOX83 is a novel 198 bp gene fragment. The nucleic acid was initially identified in a cloned fragment having the following sequence:

| (SEQ ID NO:99) | |
|---|---|
| 1 | GCCGGCCAAGGGACAGCAAACAATGCCCCTCCTCCCCTGCTCCTGCTGTGCAGACAAGGCCTCCATCCCTCCATCCTAGC |
| 81 | AGGGGTTGTGGAAGCAGGGGACCTGTCGGGCTGCAGGGAGCATAGCTGGCTCAGCATAGTTCACAGGAAGTGCCATGCTT |
| 161 | ACGCACTTCGGAAGAGACCCCAGTGGATCAGGGTCATGA |

CARDIOTOX84

CARDIOTOX84 is a novel gene fragment. The nucleic acid was initially identified in a cloned fragment having the following sequence:

| (SEQ ID NO:100) | |
|---|---|
| 1 | GAATTCACCAACATGATGATGAAGGGGGGGAACAAAGTTCTGGCCCGATCACTCATCGCCCAGACTCTGOAAGCCGTGAA |
| 81 | AAGGAAGCAGTTTGAAAAGTACCGTGCGGCCTCAGCAGAGGAACAGGCAACCATTGAACGGAACCCCTACAAGATCTTCC |
| 161 | ACGAGGCACTGAGAAACTGTGAGCCTGTGATTGGGTTGGTGCCTATCCTCAAAGGGGGTCATTTCTACCAGGTCCCTGTG |
| 241 | CCTCTGGCTGACCGACGCCGGCGCTTCCTGGCCATGAAGTGGATGATCA |

The cloned sequence was assembled into a contig resulting in the following 730 bp consensus sequence:

| (SEQ ID NO:101) | |
|---|---|
| 1 | TTTTTTTTTTTTTTTTTTCAAGTGTTTCACTTTTATTAGTGGTAATATGTGTATATATGTTTTGTCTGCACATGTGTCTG |
| 81 | TATACCATGTGTATACCACAACGGTCAGAAGTTGTCTTTGGAACGGGAGTTACAGGTGGTTAGTGAGTCTCCACGGGCTG |
| 161 | CTGGGAATCAAACCAGGTCCTTTGGAAAGAGCAGTGCTTTTCACCACTGAGCCATCTCTCCAGCCCCTCGAGTGGTCTCT |
| 241 | TGTGGCAGTGTGTCCTTTCCCCACCTCTCCTTTCCTGCTACCACCAGCGGTAGTGGGCCAGGGCACGGTTGGCCTCAGCC |
| 321 | ATCTTATGCATATTGTGCTTCCTCTTGATCACGGGACCCCTGTTGTGAAAAGCCTCCAGCAGCTCATGCGACAGCTTCTC |
| 401 | TGGCATCAGCATCCGTCGAGGCTTGTTCTCTCGGCACTCTGTGATCATCCACTTCATGGCCAGGAAGCGCCGGCGTCGGT |
| 481 | CAGCCAGAGGCACAGGGACCTGGTAGAAATGACCCCCTTTGAGGATAGGCACCAACCCAATCACAGGCTCACAGTTTCTC |
| 561 | AGTGCCTCGTGGAAGATCTTGTAGGGGTTCCGTTCAATGGTTGCCTGTTCCTCTGCTGAGGCCGCACGGTACTTTTCAAA |
| 641 | CTGCTTCCTTTTCACGGCTTCCAGAGTCTGGGCCATGAGTGATCGGGCCAGAACTTTGTTCCCCCCCTTCATCATCATGT |
| 721 | TGGTGAATTC |

CARDIOTOX85

CARDIOTOX85 is a novel 294 bp gene fragment. The nucleic acid was initially identified in a cloned fragment having the following sequence:

| (SEQ ID NO:102) | |
|---|---|
| 1 | GAATTCTATCTTCCACTGCCCCGCCCAGGCCCAGGCTGCAGTAGCCCAGTGCTTTGAACCGGAAGCAGCAACACTTTGGA |
| 81 | CAACCCAGCAGCTACTTTTTGCAGCTGCCACAGGCCATGGAGCTGAACCGAGACCACATGATCCGTAGCCTGCAGTCAGT |
| 161 | GGGCCTCAAGCTCTGGATCTCCCAGGGGAGCTACTTCCTCATTGCAGACATCTCAGACTTCAAGAGCAAGATGCCTGACC |
| 241 | TGCCTGGAGCTGAGGATGAGCCTTATGACAGACGCTTTGCCAAGTGGATGATCA |

CARDIOTOX111

CARDIOTOX111 is a novel gene fragment. The nucleic acid was initially identified in a cloned fragment having the following sequence:

| (SEQ ID NO:103) | |
|---|---|
| 1 | CGGCCGCATCACCCTGGAAGAGTATCGAAATGTGGTGGAGGAACTGCTCTCTGGAAATCCTCACATCGAGAAGGAGTCAG |
| 81 | CTCGGTCCATCGCCGACGGAGCCATGATGGAGGCTGCCAGCGTGTGCGTGGGACAGATGGAACCGGACCAGGTGTACGAG |
| 161 | GGGATCACCTTTGAGGACTTCCTGAAGATCT |

The cloned sequence was assembled into a contig resulting in the following 593 bp consensus sequence:

| (SEQ ID NO:104) | |
|---|---|
| 1 | TGCGTAAGGGGTCCAGCGGCCTGGCCGATGAGATCAACTTCGAGGACTTCCTGACTATCATGTCCTACTTCCGGCCCATT |
| 81 | GACACTACCCTGGGTGAGGAACAAGTGGAGCTGTCTCGGAAGGAGAAGCTGAAATTTCTGTTCCATATGTATGACTCGGA |
| 161 | CAGTGACGGCCGCATCACCCTGGAAGAGTATCGAAATGTGGTGGAGGAACTGCTCTCTGGAAATCCTCACATCGAGAAGG |
| 241 | AGTCAGCTCGGTCCATCGCCGACGGAGCCATGATGGAGGCTGCCAGCGTGTGCGTGGGACAGATGGAACCGGACCAGGTG |
| 321 | TACGAGGGGATCACCTTTGAGGACTTCCTGAAGATCTGGCAGGGCATCGACATCGAGACCAAGATGCACATCCGCTTCCT |
| 401 | CAACATGGAGACCATTGCCCTCTGCCACTGATCGTGCAGGGGAGGGGGTGGCTAAGGACCGAGGTTCAGCCCTTTGTCTG |
| 481 | GGCTGCTGTGACAATCAGTAACCCTTCAGTTAGCCTCCTTGTGTGGTGTGGCGTGTGGGACTCCGATGTTTTTATCTCTA |
| 561 | ATGGTGACAATAAAGGTTTCCTAATGAGCCCGG |

CARDIOTOX112

CARDIOTOX112 is a novel 179 bp gene fragment. The nucleic acid was initially identified in a cloned fragment having the following sequence:

| (SEQ ID NO:105) | |
|---|---|
| 1 | GGATCCCAGCGGATAGTACACCTATCACTGGACACATCCGCGATTTTCAGGTTTCTTACGGGACCAGGCTTATCCAAAAC |
| 81 | ATTGACAGTCGCATAGGCCACAAAACTGCCAGCTGGGTTAGTTGCTGTGACTACATATTTACCGCCATCACTTCGCTTCG |
| 161 | CTTTGGTAAGGGAGAATTN |

CARDIOTOX113

CARDIOTOXI 13 is a novel gene fragment. The nucleic acid was initially identified in two cloned fragments having the following sequences:

| | | |
|---|---|---|
| 1 | NAATTTTGAACGTGACACAAGCTCGAGTAGCATCTAGCTTGCCAATGGCTGTGATCCCATTTTTGACAGCAAACCTGTCC | (SEQ ID NO:106) |
| 81 | TACCATAGTTTTGTAAGTTTACCTTTGAGTACAGGTAATTTGAACTGTGAAATCTGTACGACAACACGGGGTGCACTAGT | |
| | and: | |
| 1 | TCTAGACAATATAAACTCCTCATAAAGGCCCTTCAGTTACCTGAACCTGATTTAGAAATTCAATGATTTGAAGCAAATAT | (SEQ ID NO:107) |
| 81 | GTACA | |

The cloned sequence was assembled into a contig resulting in the following 700 bp consensus sequence:

| (SEQ ID NO:108) | |
|---|---|
| 1 | TTTTTTTTTTTTTTTTTAATTTTCAACATTTTATTTTTGTACATATTTGCTTCAAATCATTGAATTTCTAAATCAGGTTC |
| 81 | AGGTAACTGAAGGGCCTTTATGAGGAGTTTATATTGTCTAGACCCAAGATATGCTGCAAAAGCACTCTGAAGTAAAGTAG |
| 161 | GAAATAACATTTTTCTAAAGACAGGCTTAGAAATAGTAATCCAGTAATTGAAGATGTTTCCCCTCTGTGGTAGAGGACTT |
| 241 | GATTCATACCTGGCAGCAAGGCCCCCATTCACOGGTATAGCCAAAAGGATGGOGTACAGACCACCCAGAACAAAACCAAC |
| 321 | TAGTGCACCCCGTGTTGTCGTACAGATTTCACAGTTCAAATTACCTGTACTCAAAGGTAAACTTACAAAACTATGGTAGG |
| 401 | ACAGGTTTGCTGTCAAAAATGGGATCACACCCATTCGCAAGCTAGATGCTAGTCGAGCTTCTGTCACGTTCAAAATTCGC |
| 481 | CGAAATAGACTGTTTGCTATTAGGCCCCCAAAAGCAGCATTAAGTCCAATATATGCTGATCCATATTCAAGCAGATTCCT |
| 561 | GTCTGATTCTGGAAGTTGTTTGATTTTTCTGGGTATGATATTAAATATTAAATCATCTTTGTTAGTACTTGGTTTATGAC |
| 641 | TTTCCATCTTGGACCACTCGGCAGAATGACGGCACGTTATGGCCGCCTCCCGCGCCCGCA |

CARDIOTOX120

CARDIOTOX120 is a novel 200 bp gene fragment. The nucleic acid was initially identified in a cloned fragment having the following sequence:

| (SEQ ID NO:109) | |
|---|---|
| 1 | CCATGGCCGTGGGCTTTGTGATGTGGTCCTTGATGCTCTGCACCACCCCCACAAGGGATGAGGTGGCCAGGGCAGCCACG |
| 81 | CTGTAGTTGCTGGGGCAAGCTCTGGAGTCAGATATGTAGCCATTGGTGGTCTGGAAGCACCTCTGCCAAGGATCCCAACA |
| 161 | GAAATCCATCTGCTTGTCCTTGCCAGCAACATGGTCCGGA |

CARDIOTOX130

CARDIOTOX 130 is a novel gene fragment. The nucleic acid was initially identified in a cloned fragment having the following sequence:

| (SEQ ID NO:110) | |
|---|---|
| 1 | TCATGAGGAAAGAGGTCATGCAGGAAGTGGCCCAGCTCAGCCAGTTTGATGAAGAACTCTATAAGGTGATTGGCAAGGGC |
| 81 | AGCGAAAAGAGCGATGACAGCTCCTATGACGAGAAGTACTTGATTGCCACCTCAGAACAGCCCATCGCAGCTCTGCACCG |
| 161 | GGACGAGTGGCTGCGGCCAGAGGATCTGCCCATCAAGTACGCCGGC |

The cloned sequence was assembled into a contig resulting in the following 572 bp consensus sequence:

| (SEQ ID NO:111) | |
|---|---|
| 1 | CCGACTCCTCGTTGATGAGCCATCCAGAAGTNTGATGGGGAGCGGGTAAAGCTGGAAGCAGAGCGATTTTGAGAACCTCC |
| 81 | GAGAGATTGGGAACCTTCTACACCCCTCTNTGCCCATTAGTAACGATGAGGATGCAGACAACAAAGTAGAGCGTATTTGG |
| 161 | GGTGATTGTACAGTCAGAAAGAAGTATTCCCATGTGGACCTGGTGGTGATGGTGGATGGCTTTGAAGGCGAAAAGGGAGC |
| 241 | CGTGGTGGCTGGTAGTCGGGGGTACTTCCTGAAGGGGGTTCCTGGTGTTCCTGGAGCAGGCACTTATCCAGTATGCACTG |
| 321 | CGCACCTTGGGAAGCCGAGGCTACACTCCAATCTACACNCCCTTCTTCATGAGGAAAGAGGTCATGCAGGAAGTGGCCCA |
| 401 | GCTCAGCCAGTTTGATGAAGAACTCTATAAGGTGATTGGCAAGGGCAGCGAAAAGAGCGATGACAGCTCCTATGACGAGA |
| 481 | AGTACTTGATTGCCACCTCAGAACAGCCCATCGCAGCTCTGCACCGGGACGAGTGGCTGCGGCCAGAGGATCTGCCCATC |
| 561 | AAGTACGCCGGC |

CARDIOTOX132

CARDIOTOX132 is a novel gene fragment. The nucleic acid was initially identified in a cloned fragment having the following sequence:

| (SEQ ID NO:112) | |
|---|---|
| 1 | GCTAGCCGGCTGATGAACGAGAGAGATTACTGGCCAGGGTATGGAGAAGGGAACACTTGGTGTCCAGGAGCTCTTCCAGA |
| 81 | CCCTGAGATTGTAAGGATGGTTGAAGCTCGACAGTCTCTCCGTGAGGGGTACACAGAAGATGGTGAGCAACCGCAAGGCA |
| 161 | AAGGGAGCTTCCCAGCCATGATCA |

The cloned sequence was assembled into a contig resulting in the following 325 bp consensus sequence:

| (SEQ ID NO:113) | |
|---|---|
| 1 | ACTAGTGTCTACCGCACACCTTTAAATCTAACCTTGAAGATTCTGTGGCAGCCATGGGTGGGACCGACGGCAAAGAAGA |
| 81 | CGGCGAACAGTTTAATCCGTTCTCCATTGGGACATGAAGTCCAAGGCCGGAGCGGGGGCGGCTAGCCGGCTGATGAACG |
| 161 | AGAGAGATTACTGGCCAGGGTATGGAGAAGGGAACACTTGGTGTCCAGGAGCTCTTCCAGACCCTGAGATTGTAAGGATG |
| 241 | GTTAAGCTCGACAGTCTCTCCGTGAGGGGTACACAGAAGATGGTGAGCAACCGCAAGGCAAAGGGAGCTTCCCAGCCAT |
| 321 | GATCA |

CARDIOTOX133

CARDIOTOX133 is a novel 337 bp gene fragment. The nucleic acid was initially identified in a cloned fragment having the following sequence:

| (SEQ ID NO:114) | |
|---|---|
| 1 | CCTAGGAAACATTGGAGCCTTAAGGCGGGCTACAGACAAGAACAGTTTAGCCATGCGGGTCATTCTTCACTGTTTGGCAA |
| 81 | CCTTACTTTTTTCCCTCTCTGCCTTCCTGTGTCTTGCATTCCATTTGTGGGACTGTATTTGAAAGGCCAGGCATGTAAAT |
| 161 | TCCATTAGAGCAAGGTCTCTCCTGGAATGGAACGAATCATTGACTCAATCTTTCTCTTTTCCCAGGAAGTGTCAAAATAA |
| 241 | CTCTCCGAGCAGCTGCAGCTTAGGAGGAACGGTTGTGAGACCGTCCAGCAGCTATCTTCCACCACTCAGGGTTGTCGCTC |
| 321 | ACACCCCTTAAGGATCC |

CARDIOTOX134

CARDIOTOX134 is a novel gene fragment. The nucleic acid was initially identified in a cloned fragment having the following sequence:

| | | |
|---|---|---|
| 1 | GAATTCACACAGATTGATCCTATCCTGTCTGTGAAAAGCAAGAAGTGCAGATGTGTTCATGA | (SEQ ID NO:115) |

The cloned sequence was assembled into a contig resulting in the following 535 bp consensus sequence:

| (SEQ ID NO:116) | |
|---|---|
| 1 | TCCATTTGTTGTCGTTTTTTTTTTTTTGGGTAAACAAAGGGTTAATTTATTATATAAGGTAAGCCAATAAGCTCTCATGT |
| 81 | ACTACAGAGAGAAAACATACAGTGCGCATAATAAATGAATTCCATATACTGAGAATAAATAGGATAAGCGTAGTAAAGAG |
| 161 | GAAGTCAAGAGGAGCCCACAGTTATAGCCACATGACGAGAAGTTAAAAGAAAAATAAAAGAAGAAGTCCGGGGAGAAACC |
| 241 | ACTTTATTTATTTGGAGCCATGCACTTGTTTTAGTGCCAAGGGCACAGGAAGATGGACAAGATAAGGTCCTGATCACACC |
| 321 | AGATGCTTAGAAAGATCTTTCAGTGTCTAACCTACATCTAGAAGAGTCATGAGGAGTAGTGGCAGGGTGTGTGCGCCACA |
| 401 | ACCTTTGAGGAAAGCGATCCTTATACACAGGGCGACCCCAACAACCCTGTCATTTTATCATGAACACATCTGCACTTCTT |
| 481 | GCTTTTCACAGACAGGATAGGATCAATCTGTGTGAATTCGATTTGGGTATATCGA |

CARDIOTOX138

CARDIOTOX138 is a novel 378 bp gene fragment. The nucleic acid was initially identified in a cloned fragment having the following sequence:

| (SEQ ID NO:117) | |
|---|---|
| 1 | AAATTGCGATTAGGGTAGCTATACATGGAGGGCAAGCAGGGCAGTGCTTGGTAGGTGGTGCCGCCTCGCGCGTATGTCTG |
| 81 | TAGAAACAGTTGCTTATAGGGGCCAAACTGGACTACTCCCACCTGGTCATGAAGAAGCCGCATAGCTGTTTCAAAAGAAC |
| 161 | CTGCCAGGATGTGATCCACTGGAAGCTGGGAGTTATTGCACCAGATTTGAGTTGGGCTTGTTCCCTTGGTTGGGGGCACA |
| 241 | AAGAAACCGTCTTCAGCACCACCGGCAACCCCAGAGGGTACATCCAGCTCAGGTGGGAGCTCCAAATCTTCTTCTACGTC |
| 321 | CCAGCCACCTCCTTCCTCTTGTCCCTTGCCGAGAGTATCCTCCCCCAAACCTTCCGGA |

General Methods

The CARDIOTOX nucleic acids and encoded polypeptides can be identified using the information provide above. In some embodiments, the CARDIOTOX nucleic acids and polypeptide correspond to nucleic acids or polypeptides which include the various sequences (referenced by SEQ ID NOs) disclosed for each CARIDIOTOX polypeptide.

In its various aspects and embodiments, the invention includes providing a test cell population which includes at least one cell that is capable of expressing one or more of the sequences CARDIOTOX 1-210. By “capable of expressing” is meant that the gene is present in an intact form in the cell and can be expressed. Expression of one, some, or all of the CARDIOTOX sequences is then detected, if present, and, preferably, measured. Using sequence information provided by the database entries for the known sequences, or the sequence information for the newly described sequences, expression of the CARDIOTOX sequences can be detected (if present) and measured using techniques well known to one of ordinary skill in the art. For example, sequences within the sequence database entries corresponding to CARDIOTOX sequences, or within the sequences disclosed herein, can be used to construct probes for detecting CARDIOTOX RNA sequences in, e.g., northern blot hybridization analyses or methods which specifically, and, preferably, quantitatively amplify specific nucleic acid sequences. As another example, the sequences can be used to construct primers for specifically amplifying the CARDIOTOX sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

Expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the CARDIOTOX sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING® methods as described in U.S. Pat. No. 5,871,697 and in Shimkets et al., Nat. Biotechnol. 17:798-803.

In various embodiments, the expression of one or more sequences encoding genes of expressed in distinct gene profiles based on specific serotonin modulators, as listed in Table 1, is compared. These gene profile include, e.g., “Dexfenfluramine Modulated Only” (such as, CARDIOTOX 1-9), “Fenfluramine Modulated Only” (CARDIOTOX 10-18), “Dexfenfluramiine and Fenfluramine Modulated Only”, (CARDIOTOX 19-44), “Dexfenfluramiine, Fenfluramine and Dihydroergotamine Modulated Only” (CARDIOTOX 45-57), and “All Serotonin Modulators” (CARDIOTOX 58-110). In some embodiments, expression of members of two or more gene profiles are compared.

In various embodiments, the expression of 2, 3, 4, 5, 6, 7,8, 9, 10, 15, 20, 25, 35, 40, 50, 100, 150 or all of the sequences represented by CARDIOTOX 1-210 are measured. If desired, expression of these sequences can be measured along with other sequences whose expression is known to be altered according to one of the herein described parameters or conditions.

The reference cell population includes one or more cells for which the compared parameter is known. The compared parameter can be, e.g., cardiotoxic agent expression status or serotonin modulating agent expression status. By “cardiotoxic agent expression status” is meant that it is known whether the reference cell has had contact with one or more cardiotoxic agents. Examples of cardiotoxic agents are, e.g., fenfluramine, dexfenluramine and dihydroergotamine. By “serotonin modulating agent expression status” is meant that it is known whether the reference cell has had contact with a serotonin modulating agent. Examples of serotonin modulating agents include, serotonin reuptake inhibitors such as fenflruamine, and sibutamine, serotonon receptor agonists such as sumatriptan or serotinergic agonist such as dihydroergotamine. Whether or not comparison of the gene expression profile in the test cell population to the reference cell population reveals the presence, or degree, of the measured parameter depends on the composition of the reference cell population. For example, if the reference cell population is composed of cells that have not been treated with a known cardiotoxic agent, a similar gene expression level in the test cell population and a reference cell population indicates the test agent is not a cardiotoxic agent. Conversely, if the reference cell population is made up of cells that have been treated with a cardiotoxic agent, a similar gene expression profile between the test cell population and the reference cell population indicates the test agent is a cardiotoxic agent.

In various embodiments, a CARDIOTOX sequence in a test cell population is considered comparable in expression level to the expression level of the CARDIOTOX sequence if its expression level varies within a factor of 2.0, 1.5, or 1.0 fold to the level of the CARDIOTOX transcript in the reference cell population. In various embodiments, a CARDIOTOX sequence in a test cell population can be considered altered in levels of expression if its expression level varies from the reference cell population by more than 1.0, 1.5, 2.0 or more fold from the expression level of the corresponding CARDIOTOX sequence in the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations.

In some embodiments, the test cell population is compared to multiple reference cell populations. Each of the multiple reference populations may differ in the known parameter. Thus, a test cell population may be compared to a first reference cell population known to have been exposed to a cardiotoxic agent, as well as a second reference population known have not been exposed to a cardiotoxic agent.

The test cell population that is exposed to, i.e., contacted with, the test agent, e.g., cardiotoxic agent or seotonin modulating agent, can be any number of cells, i.e., one or more cells, and can be provided in vitro, in vivo, or ex vivo.

In other embodiments, the test cell population can be divided into two or more subpopulations. The subpopulations can be created by dividing the first population of cells to create as identical a subpopulation as possible. This will be suitable, in, for example, in vitro or ex vivo screening methods. In some embodiments, various sub populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell, e.g., heart tissue. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions (e.g., cardiotoxic agent expression status) is known.

The test agent can be a compound not previously described or can be a previously known compound but which is not known to be a cardiotoxic agent or a serotonon modulating agent.

By “cardiotoxicity” is meant that the agent is damaging or destructive to heart when administered to a subject leads to heart damage.

By “serotonin modulating agent” is meant that the agent modulates (i.e., increases or decreases) serotonin levels or activity. Theses agents include for example, serotonin reuptake inhibitors, selective serotonin receptor agonist and non-selective sertonergic agonists.

The subject is preferably a mammal. The mammal can be, e.g., a human, non-human primate, mouse, rat, dog, cat, horse, or cow.

Screening for Toxic Agents

In one aspect, the invention provides a method of identifying toxic agents, e.g., cardiotoxic agents. The cardiotoxic agent can be identified by providing a cell population that includes cells capable of expressing one or more nucleic acid sequences homologous to those listed in Table 1. as CARDIOTOX 1-210. Preferably, the cell population includes cells capable of expressing one or more nucleic acids sequences homologous to CARDIOTX 1-57. More preferably, the cell population includes cells capable of expressing one or more nucleic acids sequences homologous to CARDIOTX 45-57. Most preferably, the cell population includes cells capable of expressing one or more nucleic acids sequences homologous to CARDIOTX 1-44. The sequences need not be identical to sequences including CARDIOTOX 1-210, as long as the sequence is sufficiently similar that specific hybridization can be detected. Preferably, the cell includes sequences that are identical, or nearly identical to those identifying the CARDIOTOX nucleic acids shown in Table 1.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e g., a sequence database, which assembles information about expression levels of known sequences following administration of various agents. For example, alteration of expression levels following administration of test agent can be compared to the expression changes observed in the nucleic acid sequences following administration of a control agent, such as dexfenfluramine.

An alteration in expression of the nucleic acid sequence in the test cell population compared to the expression of the nucleic acid sequence in the reference cell population that has not been exposed to the test agent indicates the test agent is a cardiotoxic agent. For example, an alteration in expression of CARDIOTOX 1-57 in the test cell population compared to the expression of the CARDIOTOX 1-57 in the reference cell population that has not been exposed to the test agent indicates the test agent is a valvulopathic agent.

The invention also includes a cardiotoxic agent identified according to this screening method.

Assessing Toxicity of an Agent in a Subject

The differentially expressed CARDIOTOX sequences identified herein also allow for the cardiotoxicity of a cardiotoxic agent to be determined or monitored. In this method, a test cell population from a subject is exposed to a test agent, i.e. a. cardiotoxic agent. If desired, test cell populations can be taken from the subject at various time points before, during, or after exposure to the test agent. Expression of one or more of the CARDIOTOX sequences, e.g., CARDIOTOX: 1-210, in the cell population is then measured and compared to a reference cell population which includes cells whose cardiotoxic agent expression status is known. Preferably, the reference cells not been exposed to the test agent.

If the reference cell population contains no cells exposed to the treatment, a similarity in expression between CARDIOTOX sequences in the test cell population and the reference cell population indicates that the treatment is non-cardiotoxic. However, a difference in expression between CARDIOTOX sequences in the test population and this reference cell population indicates the treatment is cardiotoxic.

Screening for Serotonin Modulating Agents

In one aspect, the invention provides a method of identifying serotonin modulating agents. The serotonin modulating agent can be identified by providing a cell population that includes cells capable of expressing one or more nucleic acid sequences homologous to those listed in Table 1 as CARDIOTOX 1-210. Preferably, the cell population includes cells capable of expressing one or more nucleic acids sequences homologous to CARDIOTX 58-110. The sequences need not be identical to sequences including CARDIOTOX 1-210, as long as the sequence is sufficiently similar that specific hybridization can be detected. Preferably, the cell includes sequences that are identical, or nearly identical to those identifying the CARDIOTOX nucleic acids shown in Table 1.

Expression of the nucleic acid sequences in the test cell population is then compared to the expression of the nucleic acid sequences in a reference cell population, which is a cell population that has not been exposed to the test agent, or, in some embodiments, a cell population exposed the test agent. Comparison can be performed on test and reference samples measured concurrently or at temporally distinct times. An example of the latter is the use of compiled expression information, e.g., a sequence database, which assembles information about expression levels of known sequences following administration of various agents. For example, alteration of expression levels following administration of test agent can be compared to the expression changes observed in the nucleic acid sequences following administration of a control agent, such as fluoxetine.

An alteration in expression of the nucleic acid sequence in the test cell population compared to the expression of the nucleic acid sequence in the reference cell population that has not been exposed to the test agent indicates the test agent is a serotonin modulating agent.

The invention also includes a serotonin modulating agent identified according to this screening method, and a pharmaceutical composition which includes the serotonin modulating agent.

CARDIOTOX Nucleic Acids

Also provided in the invention are novel nucleic acid comprising a nucleic acid sequence selected from the group consisting of CARDIOTOX:1-7,10-13, 19-34, 45-53, 58-85, 111-113, 120, 130, 132-134 and 138 or its complement, as well as vectors and cells including these nucleic acids.

Thus, one aspect of the invention pertains to isolated CARDIOTOX nucleic acid molecules that encode CARDIOTOX proteins or biologically active portions thereof. Also included are nucleic acid fragments sufficient for use as hybridization probes to identify CARDIOTOX-encoding nucleic acids (e.g., CARDIOTOX mRNA) and fragments for use as polymerase chain reaction (P CR) primers for the amplification or mutation of CARDIOTOX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt) or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CARDIOTOX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of any of CARDIOTOX:1-7, 10-13, 19-34,45-53, 58-85, 111-113, 120, 130, 132-134 and 138, or a complement of any of these nucleotide sequences, can be isolated using standard molecular biology techniques and the sequence information provided herein. Using all or a portion of these nucleic acid sequences as a hybridization probe, CARDIOTOX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to CARDIOTOX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nt and as many as 50 nt, preferably about 15 nt to 30 nt. They may be chemically synthesized and may be used as probes.

In another embodiment, an isolated nucleic acid molecule of the invention comprises a nucleic acid molecule that is a complement of the nucleotide sequence shown in CARDIOTOX: δ 1-7, 10-13, 19-34, 45-53, 58-85, 111, 113, 120, 130, 132-134 and 138. In another embodiment, an isolated nucleic acid molecule of the invention comprises a nucleic acid molecule that is a complement of the nucleotide sequence shown in any of these sequences, or a portion of any of these nucleotide sequences. A nucleic acid molecule that is complementary to the nucleotide sequence shown in CARDIOTOX:1-7, 10-13, 19-34, 45-53, 58-85, 111-113, 120, 130, 132-134 and 138 is one that is sufficiently complementary to the nucleotide sequence shown, such that it can hydrogen bond with little or no mismatches to the nucleotide sequences shown, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of CARDIOTOX:1-7, 10-13, 19-34, 45-53, 58-85, 111-113, 120,130, 132-134 and 138 e.g., a fragment that can be used as a probe or primer or a fragment encoding a biologically active portion of CARDIOTOX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a CARDIOTOX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a CARDIOTOX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding a human CARDIOTOX protein. Homologous nucleic acid sequences include those nucleic acid sequences that encode conservative amino acid substitutions (see below) in a CARDIOTOX polypeptide, as well as a polypeptide having a CARDIOTOX activity. A homologous amino acid sequence does not encode the amino acid sequence of a human CARDIOTOX polypeptide.

The nucleotide sequence determined from the cloning of human CARDIOTOX genes allows for the generation of probes and primers designed for use in identifying and/or cloning CARDIOTOX homologues in other cell types, e.g., from other tissues, as well as CARDIOTOX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of a nucleic acid comprising a CARDIOTOX sequence, or an anti-sense strand nucleotide sequence of a nucleic acid comprising a CARDIOTOX sequence, or of a naturally occurring mutant of these sequences.

Probes based on human CARDIOTOX nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a CARDIOTOX protein, such as by measuring a level of a CARDIOTOX-encoding nucleic acid in a sample of cells from a subject e.g., detecting CARDIOTOX mRNA levels or determining whether a genomic CARDIOTOX gene has been mutated or deleted. “A polypeptide having a biologically active portion of CARDIOTOX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of CARDIOTOX” can be prepared by isolating a portion of CARDIOTOX:1-7, 10-13, 19-34, 45-53, 58-85, 111-113, 120,130, 132-134 and 138, that encodes a polypeptide having a CARDIOTOX biological activity, expressing the encoded portion of CARDIOTOX protein (e.g., by recombinant expression in vitro) and assessing the activity of the encoded portion of CARDIOTOX. For example, a nucleic acid fragment encoding a biologically active portion of a CARDIOTOX polypeptide can optionally include an ATP-binding domain. In another embodiment, a nucleic acid fragment encoding a biologically active portion of CARDIOTOX includes one or more regions.

CARDIOTOX Variants

The invention further encompasses nucleic acid molecules that differ from the disclosed or referenced CARDIOTOX nucleotide sequences due to degeneracy of the genetic code. These nucleic acids thus encode the same CARDIOTOX protein as that encoded by nucleotide sequence comprising a CARDIOTOX nucleic acid as shown in, e.g., CARDIOTOX:1-7, 10-13, 19-34, 45-53, 58-85, 111-113, 120, 130, 132-134 and 138 48

In addition to the rat CARDIOTOX nucleotide sequence shown in CARDIOTOX:1-7, 10-13, 19-34, 45-53, 58-85, 111-113, 120,130,132-134 and 138, it will be appreciated by those skilled in the art that DNA sequence polymorphisms that lead to changes in the amino acid sequences of a CARDIOTOX polypeptide may exist within a population (e.g., the human population). Such genetic polymorphism in the CARDIOTOX gene may exist among individuals within a population due to natural allelic variation. As used herein, the terms “gene” and “recombinant gene” refer to nucleic acid molecules comprising an open reading frame encoding a CARDIOTOX protein, preferably a mammalian CARDIOTOX protein. Such natural allelic variations can typically result in 1-5% variance in the nucleotide sequence of the CARDIOTOX gene. Any and all such nucleotide variations and resulting amino acid polymorphisms in CARDIOTOX that are the result of natural allelic variation and that do not alter the functional activity of CARDIOTOX are intended to be within the scope of the invention.

Moreover, nucleic acid molecules encoding CARDIOTOX proteins from other species, and thus that have a nucleotide sequence that differs from the human sequence of CARDIOTOX:1-7, 10-13, 19-34,45-53,58-85, 111-113, 120, 130, 132-134 and 138, are intended to be within the scope of the invention. Nucleic acid molecules corresponding to natural allelic variants and homologues of the CARDIOTOX DNAs of the invention can be isolated based on their homology to the human CARDIOTOX nucleic acids disclosed herein using the human cDNAs, or a portion thereof, as a hybridization probe according to standard hybridization techniques under stringent hybridization conditions. For example, a soluble human CARDIOTOX DNA can be isolated based on its homology to human membrane-bound CARDIOTOX. Likewise, a membrane-bound human CARDIOTOX DNA can be isolated based on its homology to soluble human CARDIOTOX.

Accordingly, in another embodiment, an isolated nucleic acid molecule of the invention is at least 6 nucleotides in length and hybridizes under stringent conditions to the nucleic acid molecule comprising the nucleotide sequence of CARDIOTOX: 1-7, 10-13, 19-34, 45-53, 58-85, 111-113, 120, 130, 132-134 and 138. In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding CARDIOTOX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of CARDIOTOX:1-7, 10-13, 19-34, 45-53, 58-85, 111-113, 120, 130, 132-134 and 138 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of CARDIOTOX:1-7, 10-13, 19-34, 45-53, 58-85, 111-113, 120, 130, 132-134 and 138 or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g., Ausubel et al (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of CARDIOTOX:1-7,10-13, 19-34, 45-53, 58-85, 111-113, 120, 130, 132-134 and 138 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo et al., 1981, Proc Natl Acad Sci USA 78: 6789-6792.

Conservative Mutations

In addition to naturally-occurring allelic variants of the CARDIOTOX sequence that may exist in the population, the skilled artisan will further appreciate that changes can be introduced into an CARDIOTOX nucleic acid or directly into an CARDIOTOX polypeptide sequence without altering the functional ability of the CARDIOTOX protein. In some embodiments, the nucleotide sequence of CARDIOTOX:1-7, 10-13, 19-34,45-53, 58-85, 111-113, 120, 130, 132-134 and 138 will be altered, thereby leading to changes in the amino acid sequence of the encoded CARDIOTOX protein. For example, nucleotide substitutions that result in amino acid substitutions at various “non-essential” amino acid residues can be made in the sequence of CARDIOTOX: 1-7, 10-13,19-34, 45-53, 58-85, 111-113, 120, 130,132-134 and 138. A “non-essential” amino acid residue is a residue that can be altered from the wild-type sequence of CARDIOTOX without altering the biological activity, whereas an “essential” amino acid residue is required for biological activity. For example, amino acid residues that are conserved among the CARDIOTOX proteins of the present invention, are predicted to be particularly unamenable to alteration.

In addition, amino acid residues that are conserved among family members of the CARDIOTOX proteins of the present invention, are also predicted to be particularly unamenable to alteration. As such, these conserved domains are not likely to be amenable to mutation. Other amino acid residues, however, (e.g., those that are not conserved or only semi-conserved among members of the CARDIOTOX proteins) may not be essential for activity and thus are likely to be amenable to alteration.

Another aspect of the invention pertains to nucleic acid molecules encoding CARDIOTOX proteins that contain changes in amino acid residues that are not essential for activity. Such CARDIOTOX proteins differ in amino acid sequence from the amino acid sequences of polypeptides encoded by nucleic acids containing CARDIOTOX: 1-7, 10-13, 19-34, 45-53, 58-85, 111-113, 120, 130, 132-134 and 138, yet retain biological activity. In one embodiment, the isolated nucleic acid molecule comprises a nucleotide sequence encoding a protein, wherein the protein comprises an amino acid sequence at least about 45% homologous, more preferably 60%, and still more preferably at least about 70%, 80%, 90%, 95%, 98%, and most preferably at least about 99% homologous to the amino acid sequence of the amino acid sequences of polypeptides encoded by nucleic acids comprising CARDIOTOX: 1-7, 10-13, 19-34,45-53, 58-85, 111-113, 120, 130, 132-134 and 138.

An isolated nucleic acid molecule encoding a CARDIOTOX protein homologous to can be created by introducing one or more nucleotide substitutions, additions or deletions into the nucleotide sequence of a nucleic acid comprising CARDIOTOX: 1-7, 10-13, 19-34, 45-53, 58-85, 111-113, 120, 130, 132-134 and 138, such that one or more amino acid substitutions, additions or deletions are introduced into the encoded protein.

Mutations can be introduced into a nucleic acid comprising CARDIOTOX:1-7, 10-13, 19-34,45-53, 58-85, 111-113, 120, 130, 132-134 and 138 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., th-reonine, valine, isoleucine) and aromatic side chains (e.g., tyrosine, phenylalanine, tryptophan, histidine). Thus, a predicted nonessential amino acid residue in CARDIOTOX is replaced with another amino acid residue from the same side chain family. Alternatively, in another embodiment, mutations can be introduced randomly along all or part of a CARDIOTOX coding sequence, such as by saturation mutagenesis, and the resultant mutants can be screened for CARDIOTOX biological activity to identify mutants that retain activity. Following mutagenesis of the nucleic acid, the encoded protein can be expressed by any recombinant technology known in the art and the activity of the protein can be determined.

In one embodiment, a mutant CARDIOTOX protein can be assayed for (I) the ability to form protein:protein interactions with other CARDIOTOX proteins, other cell-surface proteins, or biologically active portions thereof, (2) complex formation between a mutant CARDIOTOX protein and a CARDIOTOX ligand; (3) the ability of a mutant CARDIOTOX protein to bind to an intracellular target protein or biologically active portion thereof, (e.g., avidin proteins); (4) the ability to bind ATP; or (5) the ability to specifically bind a CARDIOTOX protein antibody.

In other embodiment, the fragment of the complementary polynucleotide sequence described in claim 1 wherein the fragment of the complementary polynucleotide sequence hybridizes to the first sequence.

In other specific embodiments, the nucleic acid is RNA or DNA. The fragment or the fragment of the complementary polynucleotide sequence described in claim 38, wherein the fragment is between about 10 and about 100 nucleotides in length, e.g., between about 10 and about 90 nucleotides in length, or about 10 and about 75 nucleotides in length, about 10 and about 50 bases in length, about 10 and about 40 bases in length, or about 15 and about 30 bases in length.

Antisense

Another aspect of the invention pertains to isolated antisense nucleic acid molecules that are hybridizable to or complementary to the nucleic acid molecule comprising the nucleotide sequence of a CARDIOTOX sequence or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire CARDIOTOX coding strand, or to only a portion thereof. Nucleic acid molecules encoding fragments, homologs, derivatives and analogs of a CARDIOTOX protein, or antisense nucleic acids complementary to a nucleic acid comprising a CARDIOTOX nucleic acid sequence are additionally provided.

In one embodiment, an antisense nucleic acid molecule is antisense to a “coding region” of the coding strand of a nucleotide sequence encoding CARDIOTOX. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding CARDIOTOX. The term “noncoding region” refers to 5′ and 3′ sequences which flank the coding region that are not translated into amino acids (i.e., also referred to as 5′ and 3′ untranslated regions).

Given the coding strand sequences encoding CARDIOTOX disclosed herein, antisense nucleic acids of the invention can be designed according to the rules of Watson and Crick or Hoogsteen base pairing. The antisense nucleic acid molecule can be complementary to the entire coding region of CARDIOTOX mRNA, but more preferably is an oligonucleotide that is antisense to only a portion of the coding or noncoding region of CARDIOTOX mRNA. For example, the antisense oligonucleotide can be complementary to the region surrounding the translation start site of CARDIOTOX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a CARDIOTOX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol IT or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual P-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave CARDIOTOX mRNA transcripts to thereby inhibit translation of CARDIOTOX mRNA. A ribozyme having specificity for a CARDIOTOX-encoding nucleic acid can be designed based upon the nucleotide sequence of a CARDIOTOX DNA disclosed herein. For example, a derivative of a Tetrahymena L-19 IVS RNA can be constructed in which the nucleotide sequence of the active site is complementary to the nucleotide sequence to be cleaved in a CARDIOTOX-encoding mRNA. See, e.g., Cech et al. U.S. Pat. No. 4,987,071; and Cech et al. U.S. Pat. No. 5,116,742. Alternatively, CARDIOTOX mRNA can be used to select a catalytic RNA having a specific ribonuclease activity from a pool of RNA molecules. See, e.g., Bartel et al., (1993) Science 261:1411-1418.

Alternatively, CARDIOTOX gene expression can be inhibited by targeting nucleotide sequences complementary to the regulatory region of a CARDIOTOX nucleic acid (e.g., the CARDIOTOX promoter and/or enhancers) to form triple helical structures that prevent transcription of the CARDIOTOX gene in target cells. See generally, Helene. (1991) Anticancer Drug Des. 6:569-84; Helene. et al. (1992) Ann. N.Y. Acad. Sci. 660:27-36; and Maher (1992) Bioassays 14: 807-15.

In various embodiments, the nucleic acids of CARDIOTOX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of CARDIOTOX can be used in therapeutic and diagnostic applications. For example, PNAs can be used as antisense or antigene agents for sequence-specific modulation of gene expression by, e.g., inducing transcription or translation arrest or inhibiting replication. PNAs of CARDIOTOX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of CARDIOTOX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of CARDIOTOX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. USA. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

CARDIOTOX Polypeptides

One aspect of the invention pertains to isolated CARDIOTOX proteins, and biologically active portions thereof, or derivatives, fragments, analogs or homologs thereof. Also provided are polypeptide fragments suitable for use as immunogens to raise anti-CARDIOTOX antibodies. In one embodiment, native CARDIOTOX proteins can be isolated from cells or tissue sources by an appropriate purification scheme using standard protein purification techniques. In another embodiment, CARDIOTOX proteins are produced by recombinant DNA techniques. Alternative to recombinant expression, a CARDIOTOX protein or polypeptide can be synthesized chemically using standard peptide synthesis techniques.

An “isolated” or “purified” protein or biologically active portion thereof is substantially free of cellular material or other contaminating proteins from the cell or tissue source from which the CARDIOTOX protein is derived, or substantially free from chemical precursors or other chemicals when chemically synthesized. The language “substantially free of cellular material” includes preparations of CARDIOTOX protein in which the protein is separated from cellular components of the cells from which it is isolated or recombinantly produced. In one embodiment, the language “substantially free of cellular material” includes preparations of CARDIOTOX protein having less than about 30% (by dry weight) of non-CARDIOTOX protein (also referred to herein as a “contaminating protein”), more preferably less than about 20% of non-CARDIOTOX protein, still more preferably less than about 10% of non-CARDIOTOX protein, and most preferably less than about 5% non-CARDIOTOX protein. When the CARDIOTOX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of CARDIOTOX protein in which the protein is separated from chemical precursors or other chemicals that are involved in the synthesis of the protein. In one embodiment, the language “substantially free of chemical precursors or other chemicals” includes preparations of CARDIOTOX protein having less than about 30% (by dry weight) of chemical precursors or non-CARDIOTOX chemicals, more preferably less than about 20% chemical precursors or non-CARDIOTOX chemicals, still more preferably less than about 10% chemical precursors or non-CARDIOTOX chemicals, and most preferably less than about 5% chemical precursors or non-CARDIOTOX chemicals.

Biologically active portions of a CARDIOTOX protein include peptides comprising amino acid sequences sufficiently homologous to or derived from the amino acid sequence of the CARDIOTOX protein, e.g., the amino acid sequence encoded by a nucleic acid comprising CARDIOTOX 1-20 that include fewer amino acids than the full length CARDIOTOX proteins, and exhibit at least one activity of a CARDIOTOX protein. Typically, biologically active portions comprise a domain or motif with at least one activity of the CARDIOTOX protein. A biologically active portion of a CARDIOTOX protein can be a polypeptide which is, for example, 10, 25, 50, 100 or more amino acids in length.

A biologically active portion of a CARDIOTOX protein of the present invention may contain at least one of the above-identified domains conserved between the CARDIOTOX proteins. An alternative biologically active portion of a CARDIOTOX protein may contain at least two of the above-identified domains. Another biologically active portion of a CARDIOTOX protein may contain at least three of the above-identified domains. Yet another biologically active portion of a CARDIOTOX protein of the present invention may contain at least four of the above-identified domains.

Moreover, other biologically active portions, in which other regions of the protein are deleted, can be prepared by recombinant techniques and evaluated for one or more of the functional activities of a native CARDIOTOX protein.

In some embodiments, the CARDIOTOX protein is substantially homologous to one of these CARDIOTOX proteins and retains its the functional activity, yet differs in amino acid sequence due to natural allelic variation or mutagenesis, as described in detail below.

In specific embodiments, the invention includes an isolated polypeptide comprising an amino acid sequence that is 80% or more identical to the sequence of a polypeptide whose expression is modulated in a mammal to which cardiotoxic agent is administered.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of a DNA sequence comprising CARDIOTOX: δ 1-7, 10-13, 19-34, 45-53, 58-85, 111-113, 120, 130, 132-134 and 138.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides CARDIOTOX chimeric or fusion proteins. As used herein, an CARDIOTOX “chimeric protein” or “fusion protein” comprises an CARDIOTOX polypeptide operatively linked to a non-CARDIOTOX polypeptide. A “CARDIOTOX polypeptide” refers to a polypeptide having an amino acid sequence corresponding to CARDIOTOX, whereas a “non-CARDIOTOX polypeptide” refers to a polypeptide having an amino acid sequence corresponding to a protein that is not substantially homologous to the CARDIOTOX protein, e.g., a protein that is different from the CARDIOTOX protein and that is derived from the same or a different organism. Within an CARDIOTOX fusion protein the CARDIOTOX polypeptide can correspond to all or a portion of an CARDIOTOX protein. In one embodiment, an CARDIOTOX fusion protein comprises at least one biologically active portion of an CARDIOTOX protein. In another embodiment, an CARDIOTOX fusion protein comprises at least two biologically active portions of an CARDIOTOX protein. In yet another embodiment, an CARDIOTOX fusion protein comprises at least three biologically active portions of an CARDIOTOX protein. Within the fusion protein, the term “operatively linked” is intended to indicate that the CARDIOTOX polypeptide and the non-CARDIOTOX polypeptide are fused in-frame to each other. The non-CARDIOTOX polypeptide can be fused to the N-terminus or C-terminus of the CARDIOTOX polypeptide.

For example, in one embodiment an CARDIOTOX fusion protein comprises an CARDIOTOX domain operably linked to the extracellular domain of a second protein. Such fusion proteins can be further utilized in screening assays for compounds which modulate CARDIOTOX activity (such assays are described in detail below).

In yet another embodiment, the fusion protein is a GST-CARDIOTOX fusion protein in which the CARDIOTOX sequences are fused to the C-terminus of the GST (i.e., glutathione S-transferase) sequences. Such fusion proteins can facilitate the purification of recombinant CARDIOTOX.

In another embodiment, the fusion protein is an CARDIOTOX protein containing a heterologous signal sequence at its N-terminus. For example, a native CARDIOTOX signal sequence can be removed and replaced with a signal sequence from another protein. In certain host cells (e.g., mammalian host cells), expression and/or secretion of CARDIOTOX can be increased through use of a heterologous signal sequence.

In yet another embodiment, the fusion protein is an CARDIOTOX-immunoglobulin fusion protein in which the CARDIOTOX sequences comprising one or more domains are fused to sequences derived from a member of the immunoglobulin protein family. The CARDIOTOX-immunoglobulin fusion proteins of the invention can be incorporated into pharmaceutical compositions and administered to a subject to inhibit an interaction between a CARDIOTOX ligand and a CARDIOTOX protein on the surface of a cell, to thereby suppress CARDIOTOX-mediated signal transduction in vivo. The CARDIOTOX-immunoglobulin fusion proteins can be used to affect the bioavailability of an CARDIOTOX cognate ligand. Inhibition of the CARDIOTOX ligand/CARDIOTOX interaction may be useful therapeutically for both the treatments of proliferative and differentiative disorders, as well as modulating (e.g. promoting or inhibiting) cell survival. Moreover, the CARDIOTOX-immunoglobulin fusion proteins of the invention can be used as immunogens to produce anti-CARDIOTOX antibodies in a subject, to purify CARDIOTOX ligands, and in screening assays to identify molecules that inhibit the interaction of CARDIOTOX with a CARDIOTOX ligand.

An CARDIOTOX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An CARDIOTOX-encoding nucleic acid can be cloned into such an expression vector such that the fusion moiety is linked in-frame to the CARDIOTOX protein.

CARDIOTOX Agonists and Antagonists

The present invention also pertains to variants of the CARDIOTOX proteins that function as either CARDIOTOX agonists (mimetics) or as CARDIOTOX antagonists. Variants of the CARDIOTOX protein can be generated by mutagenesis, e.g., discrete point mutation or truncation of the CARDIOTOX protein. An agonist of the CARDIOTOX protein can retain substantially the same, or a subset of, the biological activities of the naturally occurring form of the CARDIOTOX protein. An antagonist of the CARDIOTOX protein can inhibit one or more of the activities of the naturally occurring form of the CARDIOTOX protein by, for example, competitively binding to a downstream or upstream member of a cellular signaling cascade which includes the CARDIOTOX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the CARDIOTOX proteins.

Variants of the CARDIOTOX protein that function as either CARDIOTOX agonists (mimetics) or as CARDIOTOX antagonists can be identified by screening combinatorial libraries of mutants, e.g., truncation mutants, of the CARDIOTOX protein for CARDIOTOX protein agonist or antagonist activity. In one embodiment, a variegated library of CARDIOTOX variants is generated by combinatorial mutagenesis at the nucleic acid level and is encoded by a variegated gene library. A variegated library of CARDIOTOX variants can be produced by, for example, enzymatically ligating a mixture of synthetic oligonucleotides into gene sequences such that a degenerate set of potential CARDIOTOX sequences is expressible as individual polypeptides, or alternatively, as a set of larger fusion proteins (e.g., for phage display) containing the set of CARDIOTOX sequences therein. There are a variety of methods which can be used to produce libraries of potential CARDIOTOX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential CARDIOTOX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the CARDIOTOX protein coding sequence can be used to generate a variegated population of CARDIOTOX fragments for screening and subsequent selection of variants of an CARDIOTOX protein. In one embodiment, a library of coding sequence fragments can be generated by treating a double stranded PCR fragment of a CARDIOTOX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the CARDIOTOX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of CARDIOTOX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify CARDIOTOX variants (Arkin and Yourvan (1992) PNAS 89:7811-7815; Delgrave et al. (1993) Protein Engineering 6:327-331).

Anti-CARDIOTOX Antibodies

An isolated CARDIOTOX protein, or a portion or fragment thereof, can be used as an immunogen to generate antibodies that bind CARDIOTOX using standard techniques for polyclonal and monoclonal antibody preparation. The full-length CARDIOTOX protein can be used or, alternatively, the invention provides antigenic peptide fragments of CARDIOTOX for use as immunogens. The antigenic peptide of CARDIOTOX comprises at least 8 amino acid residues of the amino acid sequence encoded by a nucleic acid comprising the nucleic acid sequence shown in CARDIOTOX:1-7, 10-13, 19-34, 45-53, 58-85, 111-113, 120, 130, 132-134 and 138 and encompasses an epitope of CARDIOTOX such that an antibody raised against the peptide forms a specific immune complex with CARDIOTOX. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of CARDIOTOX that are located on the surface of the protein, e.g., hydrophilic regions. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety.

CARDIOTOX polypeptides or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, ire., molecules that contain an antigen binding site that specifically binds (immunoreacts with) an antigen. Such antibodies include, but are not limited to, polyclonal, monoclonal, chimeric, single chain, F_{ab} and F_{(ab′)2} fragments, and an F_{ab} expression library. Various procedures known within the art may be used for the production of polyclonal or monoclonal antibodies to an CARDIOTOX protein sequence, or derivatives, fragments, analogs or homologs thereof. Some of these proteins are discussed below.

For the production of polyclonal antibodies, various suitable host animals (e.g., rabbit, goat, mouse or other mamnmal) may be immunized by injection with the native protein, or a synthetic variant thereof, or a derivative of the foregoing. An appropriate immunogenic preparation can contain, for example, recombinantly expressed CARDIOTOX protein or a chemically synthesized CARDIOTOX polypeptide. The preparation can further include an adjuvant. Various adjuvants used to increase the immunological response include, but are not limited to, Freund's (complete and incomplete), mineral gels (e.g., aluminum hydroxide), surface active substances (e.g, lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against CARDIOTOX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of CARDIOTOX. A monoclonal antibody composition thus typically displays a single binding affinity for a particular CARDIOTOX protein with which it immunoreacts. For preparation of monoclonal antibodies directed towards a particular CARDIOTOX protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

According to the invention, techniques can be adapted for the production of single-chain antibodies specific to a CARDIOTOX protein (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a CARDIOTOX protein or derivatives, fragments, analogs or homologs thereof. Non-human antibodies can be “humanized” by techniques well known in the art. See e.g., U.S. Pat. No. 5,225,539. Antibody fragments that contain the idiotypes to a CARDIOTOX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-CARDIOTOX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No.171,496; European Patent Application No. 173,494; PCT International Publication No. WO 86/01533; U.S. Pat. No. 4,816,567; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst 80:1553-1559); Morrison (1985) Science 229:1202-1207; Qi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a CARDIOTOX protein is facilitated by generation of hybridomas that bind to the fragment of a CARDIOTOX protein possessing such a domain. Antibodies that are specific for one or more domains within a CARDIOTOX protein, e.g., domains spanning the above-identified conserved regions of CARDIOTOX family proteins, or derivatives, fragments, analogs or homologs thereof, are also provided herein.

Anti-CARDIOTOX antibodies may be used in methods known within the art relating to the localization and/or quantitation of a CARDIOTOX protein (e.g., for use in measuring levels of the CARDIOTOX protein within appropriate physiological samples, for use in diagnostic methods, for use in imaging the protein, and the like). In a given embodiment, antibodies for CARDIOTOX proteins, or derivatives, fragments, analogs or homologs thereof, that contain the antibody derived binding domain, are utilized as pharmacologically-active compounds [hereinafter “Therapeutics”].

An anti-CARDIOTOX antibody (e.g., monoclonal antibody) can be used to isolate CARDIOTOX by standard techniques, such as affinity chromatography or immunoprecipitation. An anti-CARDIOTOX antibody can facilitate the purification of natural CARDIOTOX from cells and of recombinantly produced CARDIOTOX expressed in host cells. Moreover, an anti-CARDIOTOX antibody can be used to detect CARDIOTOX protein (e-g., in a cellular lysate or cell supernatant) in order to evaluate the abundance and pattern of expression of the CARDIOTOX protein. Anti-CARDIOTOX antibodies can be used diagnostically to monitor protein levels in tissue as part of a clinical testing procedure, e-g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

CARDIOTOX Recombinant Expression Vectors and Host Cells

Another aspect of the invention pertains to vectors, preferably expression vectors, containing a nucleic acid encoding CARDIOTOX protein, or derivatives, fragments, analogs or homologs thereof. As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., CARDIOTOX proteins, mutant forms of CARDIOTOX, fusion proteins, etc.).

The recombinant expression vectors of the invention can be designed for expression of CARDIOTOX in prokaryotic or eukaryotic cells. For example, CARDIOTOX can be expressed in bacterial cells such as E coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET I Id (Studier et al., GENE EXPRESSION TECHNOLOGY: METHODS IN ENZYMOLOGY 185, Academic Press, San Diego, Calif. (1990) 60-89).

One strategy to maximize recombinant protein expression in E coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:211:1-7, 10-13, 19-34,45-53,58-85, 111-113, 120,130, 132-134 and 13518). Such alteration of nucleic acid sequences of the invention can be carried out by standard DNA synthesis techniques.

In another embodiment, the CARDIOTOX expression vector is a yeast expression vector. Examples of vectors for expression in yeast S. cerevisiae include pYepSecl (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, CARDIOTOX can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilglrman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to CARDIOTOX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, CARDIOTOX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding CARDIOTOX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) an CARDIOTOX protein. Accordingly, the invention further provides methods for producing CARDIOTOX protein using the host cells of the invention. In one embodiment, the method comprises culturing the host cell of invention (into which a recombinant expression vector encoding CARDIOTOX has been introduced) in a suitable medium such that CARDIOTOX protein is produced. In another embodiment, the method further comprises isolating CARDIOTOX from the medium or the host cell.

Pharmaceutical Compositions

The CARDIOTOX nucleic acid molecules, CARDIOTOX proteins, and anti-CARDIOTOX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e g., a CARDIOTOX protein or anti-CARDIOTOX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Kits and Nucleic Acid Collections for Identifying CARDIOTOX Nucleic Acids

In another aspect, the invention provides a kit useful for examining cardiotoxicity of agents. The kit can include nucleic acids that detect two or more CARDIOTOX sequences. In preferred embodiments, the kit includes reagents which detect 3, 4, 5, 6, 8, 10, 12, 15, 20, 25, 50, 100 or all of the CARDIOTOX nucleic acid sequences.

The invention also includes an isolated plurality of sequences which can identify one or more CARDIOTOX responsive nucleic acid sequences.

The kit or plurality may include, e.g., sequence homologous to CARDIOTOX nucleic acid sequences, or sequences which can specifically identify one or more CARDIOTOX nucleic acid sequences.

Nucleotide Polymorphisms Associated with CARDIOTOX Genes

The invention also includes nucleic acid sequences that include one or more polymorphic CARDIOTOX sequences. Also included are methods of identifying a base occupying a polymorphic in an CARDIOTOX sequence, as well as methods of identifying an individualized therapeutic agent for treating serotonin modulating agent associated pathologies, e.g., valvular heart disease, pulminary hypertention, coronary vasospasm, or valvular and peripheral fibrosis based on CARDIOTOX sequence polymorphisms.

The nucleotide polymorphism can be a single nucleotide polymorphism (SNP). A SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Polymorphic sequences according to the present invention can include those shown in Table 2. Table 2 describes eleven CARDIOTOX sequences for which polymorphisms have been identified. The first column of the table lists the names assigned to the sequences in which the polymorphisms occur. The second column lists the human GenBank Accession numbers for the respective sequences. The third column lists the position in the sequence in which the polymorphic site has been found. The fourth column lists the base occupying the polymorphic site in the sequence in the database, i.e., the wildtype. The fifth column lists the alternative base at the polymorphic site. The sixth column lists any amino acid change that occurs due to the polymorphism.

The polymorphic sequence can include one or more of the following sequences: (1) a sequence having the nucleotide denoted in Table 2, column 4 at the polymorphic site in the polymorphic sequence, and (2) a sequence having a nucleotide other than the nucleotide denoted in Table 2, column 4. An example of the latter sequence is a polymorphic sequence having the nucleotide denoted in Table 2, column 5 at the polymorphic site in the polymorphic sequence.

For example, a polymorphism according to the invention includes a sequence polymorphism in the Novel gene fragment, 477 bp (98% SI to rat cDNA clone RGICF205′ end similar to peroxisomal phytanoyl-CoA alpha-hydroxylase), in which the cytosine at nucleotide 112 is replaced by tyrosine. In some embodiments the polymorphic sequence includes a nucleotide sequence of myosin light chain 2 gene having the GenBank Accession No. M22815, wherein the tyrosine at nucleotide 154 is replaced by cytosine.

In some embodiments, the polymorphic sequence includes the full length of any one of the eleven genes in Table2. In other embodiments, the polymorphic sequence includes a polynucleotide that is between 10 and 100 nucleotides, 10 and 75 nucleotides, 10 and 50 nucleotides, or 10 and 25 nucleotides in length.

TABLE 2

| | | Base | | | |
|---|---|---|---|---|---|
| | | Position of | | | Change Amino Acid |
| Confirmed Gene | Human Acc # | cSNP | Base Before | Base Aftere | Change |
| Novel gene fragment, 477 bp | AF023462 | 112 | C | T | PRO to SER |
| (98% SI to rat cDNA clone | | 172 | G | A | ASP to ASN |
| RG1CF20 5′ end similar to | | 184 | C | T | |
| peroxisomal phytanoyl-CoA | | | | | |
| alpha-hydroxylase) | | | | | |
| Cytochrome c oxidase subunit | M21575 | 41 | G | A | LEU to THR |
| IV | | | | | |
| Titin | X69490 | 10965 | T | C | PRO to SER |
| | | 11443 | C | T | |
| Protein-tyrosine phosphatase | M34668 | 1604 | T | C | |
| (LRP) | | 2351 | T | C | |
| | | 2356 | A | C | ASN to THR |
| Myosin light chain 2 (MLC2) | M22815 | 154 | T | C | |
| | | 280 | G | A | |
| | | 406 | G | T | ARG to SER |
| Adenylate kinase 3 | AB021870 | 530 | A | G | GLU to GLY |
| Novel gene fragment, 89 bp | AF068195 | 934 | G | A | |
| (93% SI to human putative | | 1193 | G | T | |
| glialblastoma cell | | | | | |
| differentiation-related protein | | | | | |
| (GBDR1) (AF068195)) | | | | | |
| Thymosin beta-4 | M17733 | 21 | G | A | |
| | | 62 | C | T | |
| | | 161 | A | C | |
| Bcl-x | U72398 | 340 | A | G | ILE to VAL |
| Novel gene fragment, 593 bp | Z08983 (from | 571 | C | T | HIS to TYR |
| (90% SI to human calcineurin | patent | 675 | C | T | |
| B-like protein (Z08983)) | database) | | | | |
| Ribophorin I | Y00281 | 560 | A | G | PHE to LEU |
| | | 1343 | T | C | |
| | | 1520 | C | A | |
| | | 2182 | T | C | |

The invention also provides a method of identifying a base occupying a polymorphic site in a nucleic acid. The method includes determining the nucleotide sequence of a nucleic acid that is obtained from a subject. The nucleotide sequence is compared to a reference sequence. Difference in the nucleotide sequence in the test sequence relative to the reference sequence indicates a polymorphic site in the nucleic acid.

Polymorphisms are detected in a target nucleic acid from an individual, e.g., a mammal, human or rodent (such as mouse or rat) being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed.

The detection of polymorphisms in specific DNA sequences, can be accomplished by a variety of methods including, e.g., restriction-fragment-length-polymorphism detection based on allele-specific restriction-endonuclease cleavage (Kan and Dozy Lancet ii:910-912 (1978)), hybridization with allele-specific oligonucleotide probes (Wallace et al. Nucl. Acids Res. 6:3543-3557 (1978)), including immobilized oligonucleotides (Saiki et al. Proc. Natl. Acad. SCI. USA, 86:6230-6234 (1969)) or oligonucleotide arrays (Maskos and Southern Nucl. Acids Res 21:2269-2270 (1993)), allele-specific PCR (Newton et al. Nucl Acids Res 17:2503-2516 (1989)), mismatch-repair detection (MRD) (Faham and Cox Genome Res 5:474-482 (1995)), binding of MutS protein (Wagner et al. Nucl Acids Res 23:3944-3948 (1995), denaturing-gradient gel electrophoresis (DGGE) (Fisher and Lernan et al. Proc. Natl. Acad. Sci. U.S.A. 80:1579-1583 (1983)), single-strand-conformation-polymorphism detection (Orita et al. Genomics 5:874-879 (1983)), RNAase cleavage at mismatched base-pairs (Myers et al. Science 230:1242 (1985)), chemical (Cotton et al. Proc. Natl. w Sci. U.S.A, 8Z4397-4401 (1988)) or enzymatic (Youil et al. Proc. Natl. Acad. Sci. U.S.A. 92:87-91 (1995)) cleavage of heteroduplex DNA, methods based on allele specific primer extension (Syvanen et al. Genomics 8:684-692 (1990)), genetic bit analysis (GBA) (Nikiforov et al. &&I Acids 22:4167-4175 (1994)), the oligonucleotide-ligation assay (OLA) (Landegren et al. Science 241:1077 (1988)), the allele-specific ligation chain reaction (LCR) (Barrany Proc. Natl. Acad. Sci. U.S.A. 88:189-193 (1991)), gap-LCR (Abravaya et al. Nucl Acids Res 23:675-682 (1995)), radioactive and/or fluorescent DNA sequencing using standard procedures well known in the art, and peptide nucleic acid (PNA) assays (Orum et al., Nucl. Acids Res, 21:5332-5356 (1993); Thiede et al., Nucl. Acids Res. 24:983-984 (1996)).

For the purposes of identifying single nucleotide polymorphisms, “Specific hybridization” or “selective hybridization” refers to the binding, or duplexing, of a nucleic acid molecule only to a second particular nucleotide sequence to which the nucleic acid is complementary, under suitably stringent conditions when that sequence is present in a complex mixture (e.g., total cellular DNA or RNA). “Stringent conditions” are conditions under which a probe will hybridize to its target subsequence, but to no other sequences. Stringent conditions are sequence-dependent and are different in different circumstances. Longer sequences hybridize specifically at higher temperatures than shorter ones. Generally, stringent conditions are selected such that the temperature is about 5° C. lower than the thermal melting point (Tm) for the specific sequence to which hybridization is intended to occur at a defined ionic strength and pH. The Tm is the temperature (under defined ionic strength, pH, and nucleic acid concentration) at which 50% of the target sequence hybridizes to the complementary probe at equilibrium. Typically, stringent conditions include a salt concentration of at least about 0.01 to about 1.0 M Na ion concentration (or other salts), at pH 7.0 to 8.3. The temperature is at least about 30° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations.

“Complementary” or “target” nucleic acid sequences refer to those nucleic acid sequences which selectively hybridize to a nucleic acid probe. Proper annealing conditions depend, for example, upon a probe's length, base composition, and the number of mismatches and their position on the probe, and must often be determined empirically. For discussions of nucleic acid probe design and annealing conditions, see, for example, Sambrook et al., or Current Protocols in Molecular Biology, F. Ausubel et al., ed., Greene Publishing and Wiley-Interscience, New York (1987).

Many of the methods described above require amplification of DNA from target samples. This can be accomplished by e.g., PCR. See generally, PCR Technology: Principles and Applications for DNA Amplification (ed. H. A. Erlich, Freeman Press, N.Y., N.Y., 1992); PCR Protocols: A Guide to Methods and Applications (eds. Innis, et al, Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes).

Other suitable amplification methods include the ligase chain reaction (LCR), (See Wu and Wallace, Genomics 4, 560 (1989), Landegren et al., Science 241, 1077 (1988)), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The invention also provides a method of selecting an individualized therapeutic agent for treating a serotonin modulating agent associated pathology, e.g., valvular heart disease, pulmonary hypertension, in a subject using CARDIOTOX polymorphisms. The therapeutic agent can be identified by providing a nucleic acid sample from the subject, determining the nucleotide sequence of at least a portion of one or more of the CARDIOTOX 1-210 and comparing the CARDIOTOX nucleotide sequence in the subject to the corresponding CARDIOTOX nucleic acid sequence in a reference nucleic acid sample. The reference nucleic acid sample is obtained from a reference individual (who is preferably as similar to the test subject as possible), whose responsiveness to the agent for treating the serotonin modulating agent associated pathology is known. The presence of the same sequence in the test and reference nucleic acid sample indicates the subject will demonstrate the same responsiveness to the agent as the reference individual, while the presence of a different sequence indicates the subject will have a different response to the therapeutic agent.

Similarly, the CARDIOTOX-associated sequence polymorphisms can be used to predict the outcome of treatment for a serotonin modulating agent associated pathology, e.g., valvular heart disease, pulmonary hypertension, in a subject. A region of a CARDIOTOX nucleic acid sequence from the subject is compared to the corresponding CARDIOTOX sequence in a reference individual whose outcome in response to the treatment for the serotonin modulating agent associated pathology is known. A similarity in the CARDIOTOX sequence in the test subject as compared to the sequence in the reference individual suggests the outcome in the subject will be the same as that of the reference individual. An altered CARDIOTOX sequence in the test and reference individual indicates the outcome of treatment will differ in the subject and reference individuals.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of screening a test agent for cardiotoxicity, the method comprising;
(a) providing a test cell population comprising a cell capable of expressing one or more nucleic acid sequences selected from the group consisting of CARDIOTOX: 1-209 and 210;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose exposure status to a cardiotoxic agent is knoown; and
(e) identifying a difference in expression levels of the CARDIOTOX sequence, if present, in the test cell population and reference cell population,
thereby screening said test agent for cardiotoxicity.

2. The method of claim 1, wherein the method comprises comparing the expression of one or more genes selected from the group consisting of CARDIOTOX 1-57 and 58.

3. The method of claim 2, wherein the method comprises comparing the expression of one or more genes selected from the group consisting of CARDIOTOX 1-43 and 44.

4. The method of claim 2, wherein the method comprises comparing the expression of one or more genes selected from the group consisting of CARDIOTOX 45-57 and 58.

5. The method of claim 2, wherein the method comprises comparing the expression of one or more genes selected from the group consisting of CARDIOTOX 19-43 and 44.

6. The method of claim 1, wherein the method comprises comparing the expression of 40 or more of the nucleic acid sequences.

7. The method of claim 1, wherein the expression of the nucleic acid sequences in the test cell, population is decreased as compared to the reference cell population.

8. The method of claim 1, wherein the expression of the nucleic acid sequences in the test cell population is increased as compared to the reference cell population.

9. The method of claim 1, wherein the test cell population is provided in vitro.

10. The method of claim 1, wherein the test cell population is provided ex vivo from a mammalian subject.

11. The method of claim 1, wherein the test cell population is provided in vivo in a mammalian subject.

12. The method of claim 1, wherein the test cell population is derived from a human or rodent subject.

13. The method of claim 1, wherein the test cell population includes a heart cell.

14. The method of claim 1, wherein said test agent is a serotonin modulating agent.

15. The method of claim 14, wherein the serotonin modulating agent is a serotonin reuptake inhibitor.

16. The method of claim 1, wherein the cardiotoxic agent is a dexfenfluramine of fenfluramime.

17. The method of claim 1, wherein cardiotoxic agent is dihydroergotamine.

18. A method of assessing the cardiotoxicity of a test agent in a subject, the method comprising:
(a) providing from the subject a test cell population comprising a cell capable of expressing one or more nucleic acid sequences selected from the group consisting of CARDIOTOX: 1-209 and 210;
(b) contacting the test cell population with a test agent;
(c) measuring expression of one or more of the nucleic acid sequences in the test cell population; and
(d) comparing the expression of the nucleic acid sequences in the test cell population to the expression of the nucleic acid sequences in a reference cell population comprising at least one cell whose exposure status to a cardiotoxic agent is known;
(e) identifying a difference in expression levels of the nucleic acid sequences, if present, in the test cell population and the reference cell population,
thereby assessing the cardiotoxicity of the test agent in the subject.

19. The method of claim 18, wherein the method comprises comparing the expression of one or more genes selected from the group consisting of CARDIOTOX 1-57 and 58.

20. The method of claim 19, wherein the method comprises comparing the expression of one or more genes selected from the group consisting of CARDIOTOX 1-43 and 44.

21. The method of claim 19, wherein the method comprises comparing the expression of one or more genes selected from the group consisting of CARDIOTOX 45-57 and 58.

22. The method of claim 19, wherein the method comprises comparing the expression of one or more genes selected from the group consisting of CARDIOTOX 19-43 and 44.

23. The method of claim 18, wherein the expression of the nucleic acid sequences in the test cell population is decreased as compared to the reference cell population.

24. The method of claim 18, wherein the expression of the nucleic acid sequences in the test cell population is increased as compared to the reference cell population.

25. The method of claim 18, wherein said subject is a human or rodent.

26. The method of claim 18, wherein the test cell population is provided ex vivo from said subject.

27. The method of claim 18, wherein the test cell population is provided in vivo from said subject.

28. A method of identifying serotonin modulating agent, the method comprising;
(a) providing a test cell population comprising a cell capable of expressing one or more nucleic acid sequences selected from the group consisting of CARDIOTOX 1-209 and 210;
(b) contacting the test cell population with a test agent;
(c) measuring expression of one or more of the nucleic acid sequences in the test cell population;
(d) comparing the expression of the nucleic acid sequences in the test cell population to the expression of the nucleic acid sequences in a reference cell population comprising at least one cell whose serotonin modulating agent expression status is known; and
(e) identifying a difference in expression levels of the CARDIOTOX sequence, if present, in the test cell population and reference cell population,
thereby identifying a serotonin modulating agent

29. The method of claim 28, wherein the method comprises comparing the expression of five or more of the nucleic acid sequences.

30. The method of claim 28, wherein the method comprises comparing the expression of 20 or more of the nucleic acid sequences.

31. The method of claim 28, wherein the method comprises comparing the expression of 25 or more of the nucleic acid sequences.

32. The method of claim 28, wherein the method further comprises comparing the expression of at least one nucleic acid sequences selected from the group consisting of ADIPO 58-109 and 110.

33. The method of claim 28, wherein the expression of the nucleic acid sequences in the test cell population is decreased as compared to the reference cell population.

34. The method of claim 28, wherein the expression of the nucleic acid sequences in the test cell population is increased as compared to the reference cell population.

35. The method of claim 28, wherein the test cell population is provided in vitro.

36. The method of claim 28, wherein the test cell population is provided ex vivo from a mammalian subject.

37. The method of claim 28, wherein the test cell is provided in vivo in a mammalian subject.

38. The method of claim 28, wherein the test cell population is derived from a human or rodent subject.

39. The method of claim 28, wherein the test cell includes a heart cell.

40. A serotonin modulating agent identified according to the method of claim 28.

41. A pharmaceutical composition comprising the serotonin modulating agent of claim 40.

42. A method of identifying a base occupying a polymorphic site in a nucleic acid, the method comprising:
(a) obtaining a nucleic acid from a subject;
(b) determining at least one portion of a region of nucleotide sequence corresponding to a contiguous region of any one CARDIOTOX nucleotide sequence listed in Table 1;
(c) comparing the determined nucleotide sequence to a reference sequence of the nucleic acid; and
(d) identifying a difference in the determined nucleic acid sequence relative to the reference sequence,
wherein a difference in the determined nucleic acid sequence indicates a polymorphic site in the nucleic acid.

43. The method of claim 42, wherein the subject suffers from or is at risk for, a pathophysiology associated with a serotonin modulator.

44. The method of claim 43, wherein the pathophysiology associated with a serotonin modulator is cardiac valvuopathy, coronary vasospasm, valvular fibrosis or peripheral fibrosis

45. The method of claim 42, wherein the presence of the polymorphic site is correlated with the presence of the pathophysiology associated with the serotonin mediated pathway.

46. The method of claim 42, wherein the nucleic acid is genomic DNA.

47. The method of claim 42, wherein the nucleic acid is cDNA.

48. A nucleic acid sequence 20-100 nucleotides in length comprising the polymorphic site identified in the method of claim 42.

49. The method of claim 42, wherein the nucleic acid is obtained from a plurality of subjects, and a base occupying one of the polymorphic sites is determined in each of the subjects.

50. The method of claim 42, wherein the subject is a human or rodent.

51. An isolated nucleic acid comprising a nucleic acid sequence selected from the group consisting of a CARDIOTOX:1-7,10-13, 19-34, 45-53, 58-85, 111-113, 120, 130, 132-134 and 138 nucleic acid, or its complement.

52. A vector comprising the nucleic acid of claim 51.

53. A cell comprising the vector of claim 52.

54. A pharmaceutical composition comprising the nucleic acid of claim 51.

55. A polypeptide encoded by the nucleic acid of claim 51.

56. A kit which detects two or more of the nucleic acid sequences selected from the group consisting of CARDIOTOX: 1-209 and 210.

57. An array which detects one or more of the nucleic acid selected from the group consisting of CARDIOTOX: 1-209 and 210.

58. A plurality of nucleic acid comprising one or more of the nucleic acid selected from the group consisting of CARDIOTOX: 1-209 and 210.


SEQUENCE LISTING (117 sequences)

SEQ ID NO: 1 - DNA, 188 bp, Rattus norvegicus
actagtgtcttcctccggtagagttctggcaggggcggggttcttggctgtcctgtggctgacgatgatgctgctgttggtgacacggggaccataccagcctttccagaactgtgtgtccttgcccccatgttgaaaaaggatgtgacggacgccaggagggtaattggagaaggtgtgggagatct

SEQ ID NO: 2 - DNA, 524 bp, Rattus norvegicus
tttttttttttttttttgatctccatcaagccaaaataggctggatttactgaaaacatttattacaacaaaatgtcagcgctgtgtgaccgagttgatttgggcttgaccaaagttgtatagggcaggggacctactcgtgggactggggacctgactgcccgctaagggcttaggtcttcccaggagccaaagctgagtatcttcctcctattactagtgtcttcctccggtagagttctggcaggggcggggttcttggctgtcctgtggctgacgatgatgctgctgttggtgacacggggaccataccagcctttccagaactgtgtgtccttgcccccatgttgaaaaaggatgtgacggacgccaggagggtaattggagaaggtgtgggagatctcttgccagctggcgtcattccactgttcgatggtcacaggcggaggctcaaaggaggctaggacaatgtaatcggcaaaggccagctgtacccggaggtgataggtacagccgcagtctgc

SEQ ID NO: 3 - DNA, 306 bp, Rattus norvegicus
agatctgcaagagatcaccacgtgtgtgctgatggctggcaggagacgttgagtcagctggcctgcaggcagatgggtttaggagaaccatctgtgactgaactggtccaagggcaggaaggccagcagtggctgaggttgcactccagctgggagaatctcaatgggagcaccctgcaggagctgctggtgcacaggcggtcctgcccaagcggaagtgagatttcccttctgtgtaccaagcaagactgtggtcgccgccctgctgcccgaatgaacaagaggatccttgggggtcggactagt

SEQ ID NO: 4 - DNA, 401 bp, Rattus norvegicus
tcatgaagtgcgacatcgacatcaggaaggacctgtacgccaacaacgtcatgtcagggggcactaccatgtaccccggtatcgctgaccgcatgcagaaggagatcacagctctggctcccagcaccatgaagatcaagatcatcgccccccctgagcgcaagtactcagtgtggatcggcggctccatcctggcctcgctgtccaccttccagcagatgtggatcaccaagcaggagtacgacgaggccggcccctccattgtgcaccgcaaatgcttctaggcgcacccgcgtctgtgtacgcgctctctctcctcaggacgacaatcgaccatcgtgctatggttgcagggtggccccatcctccgccgtggctccatcgccgccactgcagccggc

SEQ ID NO: 5 - DNA, 540 bp, Rattus norvegicus
tttttttttttttttttggagcaaaacagaatggctggctttaatgcttcaagttttccatttccttccacagggctttgtttgaaaaataacaaaatgaggtaaaacgagtgaatctatgtacacgtcaaaaacaggcgccggctgcagtggcggcgatggagccacggcggaggatggggccaccctgcaaccatagcacgatggtcgattgtcgtcctgaggagagagagcgcgtacacagacgcgggtgcgcctagaagcatttgcggtgcacaatggaggggccggcctcgtcgtactcctgcttggtgatccacatctgctggaaggtggacagcgaggccaggatggagccgccgatccacactgagtacttgcgctcagggggggcgatgatcttgatcttcatggtgctgggagccagagctgtgatctccttctgcatgcggtcagcgataccggggtacatggtagtgccccctgacatgacgttgttggcgtacaggtccttcctgatgtcgatgtcgcacttcatga

SEQ ID NO: 6 - DNA, 80 bp, Rattus norvegicus
caattgacagaatcagtgaggtcctcactagcctcaggatgtcccaaagtgctggcgaaggaacctcatccagcaagctt

SEQ ID NO: 7 - DNA, 233 bp, Rattus norvegicus
actagtgcttcaatgtcaaccgagagtaaaatgtgtttgtatgaaatgcctccatttgactagatagagctttatttggagaaagtcacatataacataattgaactttgaattatgcaatcccgtggattttagagtgctcctggagcaggtggcagtcaccactatctacttccagaacagtctcatcctttccagaaacccacactctgtctttcctctattccagatct

SEQ ID NO: 8 - DNA, 957 bp, Rattus norvegicus
  misc_feature (1)..(957): Wherein n is a or t or c or g.
tttggagctgggaaccgaacccagggccttgtgcttgctaggcaagtgctctaccactgagccaaatccccaacccctgtagtgcgccttctatactagaaagcttgaccactgagccacacctcccactagtgcttcaatgtcaaccgagagtaaaatgtgtttgtatgaaatgcctccatttgactagatagagctttatttggagaaagtcacatataacataattgaactttgaattatacaatcccgtggattttagagtgctcctggagcaggtggcagtcaccactatctacttccagaacagtctcatcctttccagaaacccacactctgtctttcctctattccagatctgttagacgagtggaattacatagtccggtcttttctgagttctgttactaagttttaaaggtttattctcaggtagcatcagtccgtaatgtattactgctgaatagtgttccgtgtatacagacaccgtgtgtgtcttcttccagcgagcagaggaactctgagctgtttctactttggggcttttgactaatgctatgaacatctgtgaaaaagttcgaaatgtttgatttagtacagaccctagtggggagctccggggtcatattatgacagcctcaattgtacttcctacagtggttttaccaccatttcctgctctcgtgngatctaggctccagcatccctcacaactttctgcctgagatgaagaggcatctgattgggatcttggtttgcatttccctaatgtctaataatctgagctttttttcatgtgttcattggctttctatgctgctttgcagaatgtttatttcaggctacagtctgcctttcagctgggttatctttctgtttttctgtaggattttttatttacggtcaactcatctcttaaagattaattggcattttttttttctcaacttgcggccg

SEQ ID NO: 9 - DNA, 282 bp, Rattus norvegicus
tccggaagatgctctacccaactctgaggtaatgaatgggccatttacttctcctcactcttccctggaaatgcctgcacccccaccagctcctcggacagtcacagatgaggaaatgaatttcgttaagacctgtcttcagaggtggcggagtgaaattgaacaggatatacaagacttaaagaattgtatctcgagcaccacccaggctattgagcagatgtactgtgatcctcttcttcgtcaggtgccttatcgcttacatgcagttcttgttcatga

SEQ ID NO: 10 - DNA, 317 bp, Rattus norvegicus
ggtaccccgctccacgtcctggccactcagccggacatggatgccttccttcaggagtgatccgaacgccatgtactctgccagggcccagtccacagtccggtttgtcacaagctctctgcgagtcttcaagatccggctcagccctccatggatggtaaagttctccacaggtacagaactggccacattcccaatgtgggtcaagatgtcctcctccaggccagtggaggggcaggtcatgctcctgggctgtccatccagggtgaaaaagccaggccagggggaatccagccagtgcttgatgtgcaagatct

SEQ ID NO: 11 - DNA, 405 bp, Rattus norvegicus
cggcctggttaggccaaaggtggttcatggggatgcaggttcttttgtccacattctggtcatggagcacatggtggcgatggctgaaggtaccccgctccacgtcctggccactcagccggacatggatgccttccttcaggagtgatccgaacgccatgtactctgccagggcccagtccacagtccggtttgtcacaagctctctgcgagtcttcaagatccggctcagccctccatggatggtaaagttctccacaggtacagaactggccacattcccaatgtgggtcaagatgtcctcctccaggccagtggaggggcaggtcatgctcctgggctgtccatccagggtgaaaaagccaggccagggggaatccagccagtgcttgatgtgcaagatct

SEQ ID NO: 12 - DNA, 148 bp, Rattus norvegicus
agatctttcacagacttgtcattcttgtcagcctctgccttttgccttaaggtttcaataatggagtgatcagggtttatctccaggtgtttctttgctgccatgtaacccattgttgagttgcctctgagggcttgagctttcatga

SEQ ID NO: 13 - DNA, 242 bp, Rattus norvegicus
  misc_feature (1)..(242): Wherein n is a or t or c or g.
agatctttcacagacttgtcattcttgtcagcctctgccttttgccttaaggtttcaataatggagtgatcagggtttatctccaggtgtttctttgctgccatgtaacccattgttgagttgcctctgagggcttgagctttcatgattctctccatgtttgctgtccagccatatgtgcttgtgacaatacagcatggggatgtcaccattcggtttgacacaaccaccttttcaacctn

SEQ ID NO: 14 - DNA, 280 bp, Rattus norvegicus
tgtacataccagagagttgattgtgtgaagaagcttctagaactaggagccagtgttgaccacggtcggtggctggataccccactgcatgctgcagcaaggcagtccagtgtggaggtcatcaatctgctcactgagtatggggctaacctgaaactcagaaactcgcagggcaaaagtgctcttgagctcgctgctcccaaaagtagtgtggagcaggcactcctgctccatgaaggtccacctgctctttctcagctctgccgcttgtgtgtccgga

SEQ ID NO: 15 - DNA, 167 bp, Rattus norvegicus
gaattccagaagatcgccatggccacagcgattggattcgctatcatggggttcatcggcttctttgtgaaactgatccacatccctattaataacattattgtgggtggctgagtctttgctcatcgtgggactggtgaaccaatgagggggtgacaagctcatga

SEQ ID NO: 16 - DNA, 348 bp, Rattus norvegicus
  misc_feature (1)..(348): Wherein n is a or t or c or g.
ncatccaggcaacttttacttcatgagcttgtcaccccctcattggttcaccagtcccacgatgagcaaagactcagccacccacaataatgttattaatagggatgtggatcagtttcacaaagaagccgatgaaccccatgatagcgaatccaatcgctgtggccatggcgatcttctggaattcttttctatcaggtttggtgcatcttttaaccagccgaatcgagtcctttacaaactgccgacttggctcgacaaactgcattacctgatccatgtttgtgggatggcggtttgagagggcagagacacgtagcctaggagagaattgagcccaacggaacn

SEQ ID NO: 17 - DNA, 347 bp, Rattus norvegicus
tctagagtcttccatccagggtctccggataatgtgaagccgagtgagcctctgccatccagcatgaagaaacgggactgagcagtctgcctgccgttcacatggtggtgaggatcgctggccccaggaaacactgtcacactgaagccactagcgtgtatccgtgtggatgtcgtgggcgaagcgtgggatttagagcagcagtggtttgttttgctttttctttcattttgttttgttttgttttgattttgctatctcattccatttttgaccaaagcttctctttaagtagtttattatggaagattgtcacactaacttaaaggggaagggacgtgtgtaca

SEQ ID NO: 18 - DNA, 553 bp, Rattus norvegicus
tttttttttttttttttcacacttgggatttttctttaatttttttagcacacaatgtacacacgtcccttcccctttaagttagtgtgacaatcttccataataaactacttaaagagaagctttggtcaaaaatggaatgagatagcaaaatcaaaacaaaacaaaacaaaatgaaagaaaaagcaaaacaaaccactgctgctctaaatcccacgcttcgcccacgacatccacacggatacacgctagtggcttcagtgtgacagtgtttcctggggccagcgatcctcaccaccatgtgaacggcaggcagactgctcagtcccgttcctcatgctggatggcagaggctcactcggcttcacattatccggagaccctggatggaagactctagagtcttgaaatcccagattgtcatggctccatcgatgccagtagtgcaaaatttgcgacaatcttgcttgtccacttcataaatagacacttgagtgatgctgttctggtgcagtgtttccaaggctgtgttgcggtcctcggtagtggccct

SEQ ID NO: 19 - DNA, 115 bp, Rattus norvegicus
agatctctcctagccaagggatgttgaaacatgaagggtaaggccagcctggtatcagttaaacttacgacaagggaacaaataccaagctggtgctgttggtcttatggctagc

SEQ ID NO: 20 - DNA, 419 bp, Rattus norvegicus
agatctgcctaaaaaagactgccctgggtggtgagctaatgtccatgacttctctggaaaggtagccctttctggattctgcctacctggtcagacaccaggggttctttttacagccagagagactcaactctaatgatatagctggggcagttacccatactctcagtcacctgggctgttcaaatggtgacactcttctagggctggggactgtgtcaagggagtcccaaggaacttctggtcagacatagcctcctgtgatttgggggttcttggcttggctgaaatcctgttatttattgctttgttccagggtggactgtcagggcttactgcttaacctgtttaaaatgagggacttcaagactacacagcatggctcttttcagtttattgcatgaaggagttacactagt

SEQ ID NO: 21 - DNA, 1294 bp, Rattus norvegicus
tttttttttttttttttatttctgaaaacaagctttatttaaataaggatttaaatacattacataacattaaaactggaagggaaaagaaaaccaaaagaccagtttgttccttcacatggcactgggcagtggcttgtattgtgttgaagcctttatagctagccataagaccaacagcaccagcttggtatttgttcccttgtcgtaagtttaactgataccaggctggccttacccttcatgtttcaacatcccttggctaggagagatctgcctaaaaaagactgccctggtggtgagctaatgtccatgacttctctggaaaggtagccctttctggattctgcctacctggtcagacaccaggggttctttttacagccagagagactcaactctaatgatatagctggggcagttacccatactctcagtcacctgggctgttcaaatggtgacactcttctagggctggggactgtgtcaagggagtcccaaggaacttctggtcagacatagcctcctgtgatttgggggttcttggcttggctgaaatcctgttatttattgctttgttccagggtggactgtcagggcttactgcttaacctgtttaaaatgagggacttcaagactacacagcatggctcttttcagtttattgcatgaaggagttacactagtccaagttaaaagcggaccccaaatgattacattatacaagctgtgaggtttttaaacttgtgacaagggacagaagggaaattctactcattgcaaggaaatcctcacttaagcttcagagagccacaagcacttaaaacccatgaaccttcagctgatcgtccttagccagtccaatctctatcaggaactggcatatgttcttgcgctggtcaccctgtagctgaattacttctccatattctggatgctcaattacagtaccattgcaggcaaatttcttcttaaacgccttcactagtttctttttatcgtaatcatcagcgatcccttggacagttgtaagggtcttcctgccgtttctctgttgaattcttatatggatataatcctcagtgccagcaggaagcaggtcatcacccttacttgcatcagcaaaggggtcgaaagagtggaggttctggatagcggacatacgatacgattccttttcctcggtggaaacggcctgcggaaggcggctgcgggagaaggcgggcgggggggacggagcgtcgggaagcgagggggctcgagggggaggcagctgagtcctcggcggcggctc

SEQ ID NO: 22 - DNA, 373 bp, Rattus norvegicus
gctagcagcaatcacttggggaagaatctgcagttgctgatggaccgggtggatgaaatgagtcaggacataatcaaatacaacacatacatgaggaacagcagtaagcagcaacagcagaaacaccagtatcagcagcgtcgccagcaggagaatatgcagcggcagagtcgaggcgagcccccgctccctgaggaggacctctccaaactcttcaagccccaccaagcccctgccaggatggactcgctgctcattgcaggccagattaacacttactgccagaacatcaaggagttcactgcccaaaacttaggcaaactcttcatggctcaggctcttcaagaatacagtaactaagaaaaggaagctt

SEQ ID NO: 23 - DNA, 723 bp, Rattus norvegicus
tttttttttttttttttttttgaacaaccaagtaactttttattattggttataaagccattacagcactaagagcacagtgcgcctctccactttgcagtacagaaacacattttccaagagtcactctggtggagtctcaacagtctgtcttctttgcaggaagcttccttttcttagttactgtattcttgaagagcctgagccatgaagagtttgcctaagttttgggcagtgaactccttgatgttctggcagtaagtgttaatctggcctgcaatgagcagcgagtccatcctggcaggggcttggtggggcttgaagagtttggagaggtcctcctcagggagcgggggctcgcctcgactctgccgctgcatattctcctgctggcgacgctgctgatactggtgtttctgctgttgctgcttactgctgttcctcatgtatgtgttgtatttgattatgtcctgactcatttcatccacccggtccatcagcaactgcagattcttccccaagtgattgctgctagcaagactgagcaattcatgcttatcagccacagcggacttcttctcaagctcccacatcaggacattggtcaaatgtgagtttttaattacaatcggcacttcttcaaacatgtgttcaaaggtgatgtttgcctttttcaatgcttccggggaaaagtccttctctttacaaacttccatcagttta

SEQ ID NO: 24 - DNA, 428 bp, Rattus norvegicus
tgatcaacagcttggcagtacttgatgtgagggactcgagttgcaccattgtctctcattcttgtgcagtgataaactggtataattcttaaatgatgtacaaacgaacaatcttttatttctaaataaaaccacatagtatttgagtttagtcctatctattggtctgaaatatcaaatacaattttcttcccctgtctagctgaagcagttgtggttttcaagtattgttttgtttattctctgtgccatatactaaactagactttaaggaatgttaaaatgtaaatggaaaatagagaagtagggcaggtccttaataatttgaagcaaagtttggatatggtaagtatcaagccagtgccttgtttaggggagaggtatttgcatatgtctacgtatatttgatggagtatgtgctggctagc

SEQ ID NO: 25 - DNA, 1324 bp, Rattus norvegicus
tttttttttttttttttcaagtttcagaaagggtttatttgacttacaattactggttaaagtccttcatttcaaggaagtcagggcaggaacttgaagcaactagttatactcatgaataaatgcatgcatggagagtgctcagcttgttcttatacattccagattcctttgtgtagagaatggtggtgcccacagtgggcggtcttcccttcacaattaacataatcaagccaatccctctaagacatgcccagggaccaagctaactgacacaatcctgcactgagaccctcttcctaggtgatgctagattgtgtcaagttgacaaagctagccagcacatactccatcaaatatacgtagacatatgcaaatacctctcccctaaacaaggcactggcttgatacttaccatatccaaactttgcttcaaattattaaggacctgccctacttctctattttccatttacattttaacattccttaaagtctagtttagtatatggcacagagaataaacaaaacaatacttgaaaaccacaactgcttcagctagacaggggaagaaaattgtatttgatatttcagaccaatagataggactaaactcaaatactatgtggttttatttagaaataaaagattgttcgtttgtacatcatttaagaattataccagtttatcactgcacaagaatgagagacaatggtgcaactcgagtccctcacatcaagtactgccaagctgttgatcataatctgtgaagtgactccttgttcatgagagcagatttttaacaagacgagtatgaaaggaaacctaggtaagctatgatgtataatcacataagctggtcctgtagctgtcaggtttttcagtaggaacggatagcaggaggtacagtagcacagtcagcctcattcaaggtcttgtcaataacaggtctgtaatccaaagtaaccttcccagtcttggtgtccacatatgagagggtgtgcttcctccagtgttccgcaaatggcttcttctgctggccctcgatgggcttggagtaatcatactcatcaatccgcaccttgtaatcttccctggcatgagctccccgtgactccttccgtgcttccgcaccatatatggtctgcagtgcgcacagcatcagattctgcagctccagcgtctccaccaggtctgtgttccagaccattcccctgtcaaacgtcttcagatgctgtaggtctccatagagctggctgactttttcacagccttcttgcagcacacttcccacacgggacacggcggcatggctctgcatcga

SEQ ID NO: 26 - DNA, 365 bp, Rattus norvegicus
tgtacatctgctggggtagagcttctctcgagcaggcactcctgactgtcccactgagtctcatttgtcttgcagcaattcttaaacacatcgctgactctcatgttgtgagcaggcaagagccatattcaaagtggcaggcttcaagacaagagtaacagatttcccagaacagcaccttttctctcagtcgagtgcagagacacatctcaaagtcagctatgcaggcacataattcaaagtgtaaaaaaggtgaaggagaaaaaatactgtatgcagaggaaggccttcaagtgtaaggcaggtaatggccgaagtaggctgtcgaggaaggaggtcggtgtgcaggtgattctgtatctaga

SEQ ID NO: 27 - DNA, 852 bp, Rattus norvegicus
tttttttttttttttttccatagaaagaagaaaaataatttattccaaaagatgtagaagtaagaaattcatcctgaaaatagagtttggtgtacatctgctggggtagagcttctctcgagcaggcactcctgactgtcccactgagtctcatttgtcttgcagcaattcttaaacacatcgctgactctcatgttgtgagcaggcaagagccatattcaaagtggcaggcttcaagacaagagtaacagatttcccagaacagcaccttttctctcagtcgagtgcagagacacatctcaaagtcagctatgcaggcacataattcaaagtgtaaaaaaggtgaaggagaaaaaatactgtatgcagaggaaggccttcaagtgtaaggcaggtaatggccgaagtaggctgtcgaggaaggaggtcggtgtgcaggtgattctgtatctagaaggcttctagctgtgacctcagtgcctgcactgtgcagcatgccttcatcctcaaggccagtgatacttcagataccagatggtttcatttttcaactgtggtccaaacagaggattgagctgcgccagaatcgcaatcagccaaaagagatagcagcaaacggaacaggtcaccaacatggtgatgataactccccggttaggacccttggggataaaccagggcacgaggaggcccacgaagccccagaacacgctcatcacgatcaaaggcacagtgaggccgtggtattccatgcctgcgaccccggagccgaaccagtccaccgcctcactctcgtcccacccggaagtgtcaacagaggctcacgtgaccggcgcgcgaaagccccacccc

SEQ ID NO: 28 - DNA, 178 bp, Rattus norvegicus
ggatcgggcacagagtttattgaggtgaccccagtgtgtctctactcctctttctcatccccgtgggtgatgatgtagcagagagacttgtagtcgatgttgcctgtcaggtccatgggtgtcagggcgaacagctgctccacctcagcaggagagaacttgtctgcctgggtcatga

SEQ ID NO: 29 - DNA, 167 bp, Rattus norvegicus
tccggaggatgcgatggccctttacaagaagatcctgaagtacaagatgttagacgagagggagatgccgggtgccgagcaaatgtgctttgagaccagcgccaagaccggacacaacgtggacctcctctttgaaaccttgttcgacctggtggtacctatgatca

SEQ ID NO: 30 - DNA, 217 bp, Rattus norvegicus
ggatccggggtgttaggaggagttgagggagcttgctgtgaaccaccttccaggttacttccgtcaattctcccattctgcatggcaagattgtgattgatttgtgcttttgtttcgtactggaaattttcaaaggtgtatttgtcagatcttctttgacgcatcttaaacagtctggcaccgcgattaccgaaatgggacaattcttctatcatga

SEQ ID NO: 31 - DNA, 68 bp, Rattus norvegicus
  misc_feature (1)..(68): Wherein n is a or t or c or g.
naatttccttcaggggtccagaatatcctggtgcaatgttctccggatttggggggcttcgtggatcc

SEQ ID NO: 32 - DNA, 130 bp, Rattus norvegicus
ccatggacacgatgtcgacggcatggatctgggcaagaaagttagcgtccccagagacatcatgatagaagaattgtcccatttcggtaatcgcggtgccagactgtttaagatgcgtcaaagaagatct

SEQ ID NO: 33 - DNA, 389 bp, Rattus norvegicus
tgatcacgacaggaatattctcagatatccacccctttggtgtcctattaaagcatcgtctgcccgaaagaggattggcaaaggccaaaaacctgggatctgttagcagcagtcgttcgaagtctggaaccttgaatttaaccatttttgatgctttctcaaaacctccaaatggagtggcaactctgttaaagctcctgtaatctggcagttctgcctttccttcaggcttgaaaagtttcgggtacaaagcttccaggagctctggatcgtcgccaatggcctgctcccagggagactggtagtacttaggaacagccgtcgtgttaaatctttcaggaggaatttccttcaggggtccagaatatcctggtgcaatgttctccgga

SEQ ID NO: 34 - DNA, 1070 bp, Rattus norvegicus
  misc_feature (1)..(1070): Wherein n is a or t or c or g.
tttttttttttttttttgagagattcttaaaccagaatttaattgttcagttcaaattgaacgccacaaaatgaaatgtgtgtaaccgcaattggatgaccacagtgacgaggcactcaaatggcttcgccgctaagaagaccgacggcagcttttatgtgtagagctctcggcggcctgcctggcttcccgttcacaagtcatctgactctggcatagtgacatcttctgcaggctcagttgtgatcacgacaggaatattctcagatatccacccctttggtgtcctattaaagcatcgtctgcccgaaagaggattggcaaaggccaaaaacctgggatctgttagcagcagtcgttcgaagtctggaaccttgaatttaaccatttttgatgctttctcaaaacctccaaatggagtggcaactctgttaaagctcctgtaatctggcagttctgcctttccttcaggcttgaaaagtttcgggtacaaagcttccaggagctctggatcgtcgccaatggcctgctcccagggagactggtagtacttaggaacagccgtcgtgttaaatctttcaggaggaaatttccttcaggggtccagaatatcctggtgcaatgttctccggatttggggggcttcgtggatccggggtgttaggaggagttgagggagcttgctgtgaaccaccttccaggttacttccgtcaattctcccattctgcatggcaagattgtgattgatttgtgcttttgtttcgtactggaaattttcaaaggtgtatttgtcagatcttctttgacgcatcttaaacagtctggcaccgcgattaccgaaatgggacaattcttctatcatgatgtctctggggacgctaactttcttgcccagatccatgccgtcgacatcgtgtccatggatttccttcgtgatggctgaagcttgctgtttcctttgcttcaccatggcactgtgtgatagcatagtttgttttttgttcccttgctgtcagactgcacttttcagcaggggtgaatcccaattgcggggagagctggaagtgtn

SEQ ID NO: 35 - DNA, 316 bp, Rattus norvegicus
  misc_feature (1)..(316): Wherein n is a or t or c or g.
tgatcagttcttaggagtgaggtaagggacctttttctctctaaaacaaaaaccccttttgggggtggccatcctaggtttccaagaatttaggaagccgggagaaggggagggcaagtcagaaggatcacaaggctggntgagtgtggtgatgcctgcaatactggcgggagggtgaggcaggagaatgcggagttcaaggccctccatggctagagctgggtagaaangaggcgaggctgcagggatcctgtctgggagatcgaatctcatagaaggggactagggttggctcgagggtctttttgattcngga

SEQ ID NO: 36 - DNA, 1143 bp, Rattus norvegicus
  misc_feature (1)..(1143): Wherein n is a or t or g or c.
tttttttttttttttttggtctttatttttctttaatgtttttctgattggcgttgccactgggagatttgaaaaagaaaaaaccaaatgaaacaagttccctgcaaggacctaggcaggcagtccagctctttggctgacaagatcggagaggatctttcaaatcctttctttgaatatttggtcaaaatggctttagtttaagtccactggtcctgtgagattgtaggtgaggctgggatgacagactggtagaaatacttgcccagcacttgtgaggccttgggttgtaactggttttcctttggtgttctgattttgttcttgaagggaaggaaaacagttatgaaaggctcccatcagccacctgtgcttctaggagtgctagaccctcctaggcagagaaatggagtcctctccccctccataatattcccatcaaaatacacagacataaataaatgtagccatcacttgatcagttcttaggagtgaggtaagggacctttttctctctaaaacaaaaacccctgttgggggtggccatcctaggtttccaagaatttaaaaagccgggagaaggcaaggccaagtcagaaggatcacaaggctggctgagtgtggtgatgcctgcaatactggcaggaggctgaggcaggagaatgcggagttcaaggccctccatggctagagctgggtagaaaccagccgaggctgcaaagatcctgtctgggagatcaaatctcatagaaaggcactagggttggctcgagggtctttttgattccggaatctcattgctagccaaacaccgaggatctctgtgaaactgaagaagagcccaacacctcctaggatcttgagagctttgtctgaatgttttaggaatgtctccccacacatctggcatggagagctcctagttttgcacagtgcactgcaggaagcatcatcatgtaggtgcacggttgtaaggttaaacaaaccacagcagtcaaaacttctctccagttcatgcctggtactgttgctcaagacccaccacgaagcattgatgacatctgcctgtgtgtttctgttaagagccagacatgagcaagagattccaaactggaagatgaagaccaaacccaggatgatcan

SEQ ID NO: 37 - DNA, 74 bp, Rattus norvegicus
gtgcactctgcagtgaggacaatagatggctcactgtggcagcctggctgagagggaactctcatgctgctagc

SEQ ID NO: 38 - DNA, 149 bp, Rattus norvegicus
agatctcggactatgctgcattctatcacaataaattcttctagctgtttaggatggcataaactattgaaaggatgactccagaaggtgttcccatcaatgtctgcaacttgtaaggtatttgggtctatgagatggatggcactagt

SEQ ID NO: 39 - DNA, 408 bp, Rattus norvegicus
tcatgatggtctggatttttattattcttcaaaacagcatgctcagaagatggtggagtttcttcagggtacagttccctgtagatacaaatcatcacaaagattgatctcccaggatattcatagtaacacatacaattacaagagtactttttctgtggaaattgttctaatatgcaaggataatgttgtctgtctgtcaccaaaactggcacagagccttggaaatatgaaccagatatgtatttgtatacgagtaactagtgccatccatctcatagacccaaataccttacaagttgcagacattgatgggaacaccttctggagtcatcctttcaatagtttatgccatcctaaacagctagaagaatttattgtgatagaatgcagcatagtccgagatct

SEQ ID NO: 40 - DNA, 139 bp, Rattus norvegicus
gtgcacgccttcgacatggaggatctgggggataaggccgtgtattgccggtgctggaggtctaaaaagttcccgttctgcgatggggctcacataaagcacaatgaggagactggagacaacgtgggacctctgatca

SEQ ID NO: 41 - DNA, 618 bp, Rattus norvegicus
tttttttttttttttttgattttggaataatttaatatataacctcaagacataactctattctaagaccattattttaaaggaacggatccttacgagaccaagataacccacagagcatgaggttggttcagcctttccttttcttcttctttcaacaaatgtgcaccacgatgtttcaatggcaaggccgatgccgtgaacatgaaagctgcgatttgcaagtaccaaccacaccagaacctgggaggccaaccagacagtgggttgggtgccattctaattaaatgatcaggtgacatcacaacacgctggggtgtagcctcgcaactgtccattaagtttcttttttcttgatgatcagaggtcccacgttgtctccagtctcctcattgtgctttatgtgagccccatcgcagaacgggaactttttagacctccagcaccggcaatacacggccttatcccccagatcctccatgtcgaaggcgtgcacaaccttcgggttgtctttctggatctgaaggttcaccatagctttggtgcgactctctttagcgtagaacttcttgtaagccaggtaaccgataacggctgtgccagcagcaaaggtcacgg

SEQ ID NO: 42 - DNA, 417 bp, Rattus norvegicus
gtgcacccttacatcagaacaaaagctactttgagttcaaaatccagtctaccggaatctggggtataggtgttgcaactcagaaagttaacttgaaccagattcctcttggccgtgacatgcatagcctggtgatgagaaatgatggagccctgtaccacaacaacgaagagaaaaacaggctgccagcaaacagccttcctcaggagggagatgtagtgggtataacatatgaccatgtagaattaaatgtatatttgaatgggaaaaacatgcattgtccagcatcaggtatacgagggaccgtgtatccagtcgtgtatgttgacgacagtgcaattttggattgccagttcagtgaattttatcatactcctccacctggttttgaaaaaatactatttgagcagcagatct

SEQ ID NO: 43 - DNA, 717 bp, Rattus norvegicus
tttttttttttttttttgtcaaacaaatactttttataagaaaaattccctttaaatatttatatacatgttaccacgtaatactgttaatcaaacccatggtttatttgtttaaataagattaaataaattgcctagatcttttaaatcaaaccttagtatggtataatggatatatgggttccttagacaacaataagaagcatgtgttcttgtctctagatcaaggagagctttatcaagtggtaagcgctgtgtgatggtgcagaagtctaagttttgaaaacaaactcattcagaagatctgctgctcaaatagtattttttcaaaaccaggtggaggagtatgataaaattcactgaactggcaatccaaaattgcactgtcgtcaacatacacgactggatacacggtccctcgtatacctgatgctggacaatgcatgtttttcccattcaaatatacatttaattctacatggtcatatgttatacccactacatctccctcctgaggaaggctgtttgctggcagcctgtttttctcttcgttgttgtggtacagggctccatcatttctcatcaccaggctatgcatgtcacggccaagaggaatctggttcaagttaactttctgagttgcaacacctataccccagattccggtagactggattttgaactcaaagtagcttttgttctgatgtaagggtgcac

SEQ ID NO: 44 - DNA, 216 bp, Rattus norvegicus
agatctaactactccaaccttcacaattccagctacttgataataataggagtaacccaatgaatactgtatggtctgaaagctactatacaatatgattcttgaggaggagggagagagggagagagggagttagagactgtcacaaagccctgggtgcttctctggagttagcagggaaacaggaccctgggcaagcagctcgggtgccctagg

SEQ ID NO: 45 - DNA, 546 bp, Rattus norvegicus
tttttttttttttttttggtgtttctctcttttatttaaaaacagtgcttcgttaccatttgcaaaggctgaggcagggcccctcctttgctaagagtttataaaagccagcaacatgatcaataatttatacacatggagagtaatacaaaaaataatgaataaaagctaaagatctaactactccaaccttcacaattccagctacttgataataataggagtaacccaatgaatactgtatggtctgaaagctactatacaatatgattcttgaggaggagggagagagggagagagggagttagagactgtcacaaagccctgggtgcttctctggagttagcagggaaacaggaccctgggcaagcagctcgggtgccctaggaggtgactctgggagaggatgggaaggaaggagacacagctgggtggtcaattggacaagcattccagtatgcccccatgtcccagaggtacctgtcctgccacagggaaaccacacgtgctaggcaagccactccctgccacagaggtgtggaggag

SEQ ID NO: 46 - DNA, 207 bp, Rattus norvegicus
tgtacaagagaaggactaagaaccaaactgtttacagagatccaagcacgagtgagagagcacactcctcacacggctttccgatgatactcaggaggagccacttcataatcactggcactgaacagagttgcagaattctttgccaggtacttgaggaaatcatgtagatagttcagtaataaagcaaggcttttctcatctaga

SEQ ID NO: 47 - DNA, 920 bp, Rattus norvegicus
tttttttttttttttttgaaatttaaagaaaaaatttattgaagatctgaaaaacaactcctacaagattgacttttccataaaactgtagctacacgatgcattgcgtctatcatgttaaaacgtgcattagacacaaatacaaaaaccatgaaaacaagccaccattctttaacaattgagcaaagataaaatgcctaaggaacaacatggatgacttgcaaaggatgggctctttaagcaccatttaaaaaaaaaaagagcacagatggatgagtgttcagttatacacactgaagggaacctttggcactaggagtcagagcattttgtcatagagcattaacacatattataaaagtgcgtagtgtcaaaggaacagaaccaccagcattcaaaagcagctttgtcaactaggcaaacactctacagcatgtctctccgttgtccatcactgatacactggtagaaactttgaaatgaaaaaaagaaagaaaaaaggagcagttaactccttttattttctctgtttaaaatcaaacaggaaacaaacatcaactctgttatacactaacggtcttcaaagtacatcatttgtacaagagaaggactaagaaccaaactgtttacagagatccaagcacgagtgagagagcacactcctcacacggctttccgatgatactcaggaggagccacttcataatcactggcactgaacagagttgcagaattctttgccaggtacttgaggaaatcatgtagatagttcagtaataaagcaaggcttttctcatctagaggtgtataggccaacatcgctccaattcgcacaaacaatctcagtaagtgtggcgctccatacacctgggacatgggtgcatccgggtgatcggccaaaatttcagcatactgtggtc

SEQ ID NO: 48 - DNA, 203 bp, Rattus norvegicus
agatctctctcctggaagacctgaaccaggtgatagagaacaggctcgagaacaagattgcttttattcgccagcacgccatcagggtccgaatccacgcccttttagttgaccgctatctgcagacttacaaggacaaaatgaccttcttcagtgacggggaactggtctttaaggacattgtggaagatcctgataaattc

SEQ ID NO: 49 - DNA, 180 bp, Rattus norvegicus
ggatcccacgccctcttctgagggtactagacatgcacaccgtgtgcagacatgcatgcaggtaaaatgtgtgctcacaaaactaaaaacctgaaaaagaaaaccaaccctgcatttgtggagtcatcacagcccatagactgtgccaacgagtgtgtgaaccagaagagaagttcatga

SEQ ID NO: 50 - DNA, 335 bp, Rattus norvegicus
tccggatgagcaacctcaccacaacatttgcattctcttccacactctccccattacagaagacggcaaatctgagaaagtcaagatatcgttctccttcaactggattccacccaatgtctgggtaacccttagacaccagcatctggcagctctgcagaccacagccggccagatagcgaaccaccttctccagatccggctctcgtagagcaagggcaagctcattgttatccatcactgacgctgcggccacgtctaatggagttgaacctctcatggctggtgaggcaagaccaacactgctgttttccagtaaataactgagatgatca

SEQ ID NO: 51 - DNA, 81 bp, Rattus norvegicus
gaattctgcgtcagtccagagacagtgaattgagtctcgataacattggtgaagctggccttagtccacctcccatccgga

SEQ ID NO: 52 - DNA, 428 bp, Rattus norvegicus
tccggatgttagttttgtcttgacagacatagctgttctccgtggtcggctgagcccagtctcgttctcagcaaatactcggaactcatattcagttgcttctagcaaacctccgatggtgaactgcctgtccttgatccgttccttattgctcttcttccaagcactgtccccagactgtctgtactcaacccagtagccaaggatttctttgccaccatcgcattcgggcttctcccactggaggatgacactgtctttggatatcgaaagaatctcgagttctcctggttggcttggcttatcgaagggatctttgcaaacgacgggttcagaagcagggctggtctcgctaaggcccacgtcattctgtgcgatgatacggaattgatattctgcgtcaggaacaaggccagtgaccgtgtaca

SEQ ID NO: 53 - DNA, 374 bp, Rattus norvegicus
ggtaccattttacatttgctttctctctggagagctggcaggagaagacagcgtcgtcaaactctgtgaccgtctggtcttccaggtgctccacgaattccgttggggcttcgatgatgagcagctctgccacggatttatcttgaccagcagtaacgatgtatccatcttcatctgggaagccacagtccttgatgattagagagtgcttgtacttgtcaatgcggtatgatatacggttgtcaaaagccacttcttccccatttttggtccacttcagggttacattgagacgattcaccttgcaccagaacgtgactgacttcttctccattgtttcaatatctttaaggggttcgataatcctaagatct

SEQ ID NO: 54 - DNA, 429 bp, Rattus norvegicus
actagtcacctcgatctgggctctctccgtgagaatgccttcagccttttcccacttcacctcaggttctgggcgacctttgatagtgacaaacaggcgcaaagtggcacttgccctcagagtgaccaccttcctgagatcagcatcgagttctatttctggggcttccatcctctcctgagccacaacagagcctggtatagttgcaggctcacccacgccttcggtattgaacgcacagatacggaagttgtactctgtgttctctttaagcttggtcactgtgaactgcttcccttgtaatcccgatggtggcgtacaggtagtccattcgtcagccgcggcttctttgagttcaatcacataggctctaacgggtgcgccaccgtcgtaaattggcttattccatgctagggagacagaagatct

SEQ ID NO: 55 - DNA, 207 bp, Rattus norvegicus
ccatggaaaatggtgtttgaggcgagggggtcggtcactgtgtccagtcccatcacaagactgggaaagcatgcatggggttcggggtttggaaaaaaggaggacagaattgattaaaattgaaatggaggattatctctaagatttagtctctgtagaattttgtttacaaatactaccaaaagggtcatgatcgggagtgctagc

SEQ ID NO: 56 - DNA, 1216 bp, Rattus norvegicus
tttttttttttttttttgaacttttgccactttgtattttattgtggaactcagtttcttttttctttttttttttcctttacatcaaatatcctcaatagaagaggggatattgcacacaaataccataaaagcactacatattactttcactggaaactaatttttctacattagatatgactggataggatggaagtgatgcaggattataagacataataccatacacagaggcagaccgacacaaacaccattcagaacaagagagagagtgagcttctccacagccgggcttaggactgcacgctgcctgcgggcgcatgcggggaaagcaaggaccgccgcggcgtgggcgggcggctgagcagagccacttctccggggctccagtttcgcgagctccacgcgtgcggagaagccgattattagctgttgtttttttttcccttccttttcagtttttgatgctgcctttgaaatgaattcttaaaagttccggatttttgaaatagtgaatagttttaataccaggtgaataaaacctaatcgctaccaaagcgcggtgctcatccctaggctgcttttggtgtgttgttcagctggttacgtgataaaagcttacagttcctctcacgtggaaacagaatctttttctcctaaatctgaagtatgaaaggaaaaaaaaaggagagaaggaacgtcattatcctaactcacaaatgtcattgccaagcagggacctcctgtgacaaatgacagaggaggtgagaaaaaacaactcctgaattgtagtgccgctccaggagctaagatttgtaacacaaatgggaggtggtaaaatttccattagcaaatgattaaatttataaaacgagtattagaaagctcctaaatttcataagctattggaaacacttaaaacattcatatacaccggggaaaccattcactatgatatgtaaggttaagaaaaaaaatttttttcttttgaattccatggaaaatggtgtttgaggcgagggggtcggtcactgtgtccagtcccatcacaagactgggaaagcatgcatggggttcggggtttggaaaaaaggaggacagaattgattaaaattgaaatggaggattatctctaagatttagtctctgtagaattttgtttacaaatactaccaaaagggtcatgatcgggagtgctagcacaatagaattc

SEQ ID NO: 57 - DNA, 105 bp, Rattus norvegicus
  misc_feature (1)..(105): Wherein n is a or t or c or g.
naatttggtttatttctctattcacttgttttcaaggcaagaaaaatgtagctaaaggaacaactagccctttcttccatttctgtctccaaattactcactagt

SEQ ID NO: 58 - DNA, 281 bp, Rattus norvegicus
tcatgactgggagactctgattcctcctcagtccacccaataaactgccaccagaatttaaatagacagcagagtctggtttttgaagacccatttctgcctctcggcttttcccattctcccggggaacaggggtcttgaccaccctggctattcccagcctcttcagcctgtccaccaagttcatcttcagctggccaacatcaggaggggcccttgaaggtctcaagccatacatttcttgcaggaatgtttcagctggtctggaagccaagaaattc

SEQ ID NO: 59 - DNA, 1115 bp, Rattus norvegicus
tttttttttttttttttgtgttgtacaaaaatacaagcttaaaaaaaactgaagttctaataatcacaaatacaaagggatctatctgggtggtgtttgggttctccgtgccccaaagtccccggataagaaagtctccatttctgatgtaaaggacaagataaaattccttattttgctaacgctgagagtgcaccattggatgggtgcatttgatcagggaccagcagggaaggcatctcccacaggctcggctcacaccactctgcgcatgcaccaactctccggaacagcctcctcccagcaacagcctgggctgccccgggtttccttcgtaggcaggcgcttccagcttgtgttctctagagacaaggtgccagcacttcggtattactgtcacgtttcgatagaatttggtttatttctctattcacttgttttcaaggcaagaaaaatgtagctaaaggaacaactagccctttcttccatttctgtctccaaattactcactagtccccacgttactagactccatcctcaaaaacctttgcggccggctctatccctcactacgccctctccacattcacaatccttctacaacatcccttttctctcaagttaggccggtcccaattctcagtgcatctatccttcatgtgctaatttatttacgaggtcagttaatgtggacccctcagtcttccttcaggatacccattttgggcgaggttgtgcaaactggggctccaaagctacccatcatgactgggagactctgattcctcctcagtccacccaataaactgccaccagaatttaaatagacagcagagtctggtttttgaagacccatttctgcctctcggcttttcccattctcccggggaacaggggtcttgaccaccctggctattcccagcctcttcagcctgtccaccaagttcatcttcagctggccaacatcaggaggggcccttgaaggtctcaagccatacatttcttgcaggaatgtttcagctggtctggaagccaagaaattctcggagacatggacacggggttcaaagggcacgggggaggaacatggtgactgcgacggaggcgcaggcagc

SEQ ID NO: 60 - DNA, 153 bp, Rattus norvegicus
tgatcaatctactgtgaaagactctcctcctgatacctgtcctccttctgtaacgaagcttacttagcttttagctgtgaaaaactctgggaacttccccacccattaattcttataaagtcaagtccccaaactggatgtgtctcagtgcac

SEQ ID NO: 61 - DNA, 89 bp, Rattus norvegicus
agatctgcagcatgaccgggcccgtctctgggtcgttcatccactgggtgctgttaagtgggttctccagcatgtcttcaaatgctagc

SEQ ID NO: 62 - DNA, 442 bp, Rattus norvegicus
cctaggaagcggaggtttagaatcttgatctgctggtcttccaggtccattcggatgatgccatcctcaccatcaatactcagaaggaccccggtagcctctcggtcctcacccagaatcactttcaccttgttgttcttggtgggggtgatgggctccagatgctcactggagatactgaccaccttctcactatctttcaggtacacggagcacatgcctcccgtgacactgcggatgacgcctgtctgccccactatttgtgtgtccagataggtgtctcgaaccttcacctggatatcagtggtcacccagtcactggagttctgctcaatgcctgagcctggtgtgtggggattgtagcctccaggagaaggagctccaggggtcattggactgtagccaacagggctggggctgggactagcctgataggccatgg

SEQ ID NO: 63 - DNA, 710 bp, Rattus norvegicus
ttttttttttttttttttttttttcaaacagtttctctttattgaaaggcctgaacacaaaggcaagctgggacagcagaaagaaggcaggacattcctcagactgctctgattcctagagtaccaggggaggaggaaaaggaaatccagagtgattgccctggcttgccccagactcggggttccatcctaggccaagcaaggccaaagcgggctgcttgctccgtgtctgcactgcacgcttgggcctcaggcctccaggagcttccctaggaagcggaggtttagaatcttgatctgctggtcttccaggtccattcggatgatgccatcctcaccatcaatactcagaaggaccccggtagcctctcggtcctcacccagaatcactttcaccttgttgttcttggtgggggtgatgggctccagatgctcactggagatactgaccaccttctcactatctttcaggtacacggagcacatgcctcccgtgacactgcggatgacgcctgtctgccccactatttgtgtgtccagataggtgtctcgaaccttcacctggatatcagtggtcacccagtcactggagttctgctcaatgcctgagcctggtgtgtggggattgtagcctccaggagaaggagctccaggggtcattggactgtagccaacagggctggggctgggactagcctgataggccatgg

SEQ ID NO: 64 - DNA, 236 bp, Rattus norvegicus
gcttatggtaaggaggctccatttctcttgtcctttcgtactgggagaaattgtaaatagatagaaaccgacctggattgctccggtctgaactcagatcacgtaggactttaatcgttgaacaaacgaaccattaatagcttctgcaccattgggatgtcctgatccaacatcgaggtcgtaaaccctaattgtcgatatgaactcttaaataggattgcgctgttatccctagg

SEQ ID NO: 65 - DNA, 1618 bp, Rattus norvegicus
tccattttgttccttcctccggttgtgccccccggttcctctttttcttttttaacctgggctaggtttatttattgtacatatatactttattgagatttttttcataaattggttgggagcacttatggtaaggaggctccatttctcttgtcctttcgtactgggagaaattgtaaatagatagaaaccgacctggattgctccggtctgaactcagatcacgtaggactttaatcgttgaacaaacgaaccattaatagcttctgcaccattgggatgtcctgatccaacatcgaggtcgtaaaccctaattgtcgatatgaactcttaaataggattgcgctgttatccctagggtaacttggtccgttgatcaataattgggtcaataagatattagtattactttgacttgtgagtctaggttaaaatcattcggaggattttttattctccgaggtcaccccaaccgaaattttttagttcatatttattttgttttagcccattaggttgtttttatataagttgaactagtaaattgaagctccatagggtcttctcgtcttattgggagattccagcctcttcactggaaggtcaatttcactgattgaaagtaagagacagttgaaccctcgtttagccattcattctagtccctaattaaggaacaagtgattatgctacctttgcacggtcaggataccgcggccgtttaactttagtcactgggcaggcaatgcctctaatacttgttatgctagaggtgatgtttttggtaaacaggcggggttcgtgtttgccgagttccttttactttttttaatctttccttaaagcacgcctgtgttgggctaacgagttagggataggtaattttattgttgggttagtacctatgattcgataattgacaatggttatccgggttgtcatacacttgtgctaggagaattggttcttgttactcatattaacagtatttcatctatgggtctatagattagcccaatttgtaatataggaatttattgaggtttgtggaattagtgtgtgtaagtatgtatgttgagcttgaacgctttctttattgatggctgcttttaagcctacaatggttaagtggttgtagttgtttattcactatttaaggttttttccttttcctaaagagctgtccctcttttggttatattttaagtttacattttgatttgttgttctgatggtaagcttaaagttgaactgaaattcttttttgggcaaccagctatcaccaagctcgataggcttttcacctctacctaaaaatcttcccactattttgctacatagacgggttgattcatgaaattgtttttaggtagctcgtttggtttcggggttcttagcttaaattctttttgttaaggattttctagttaattcattatgcaaaaggtacaaggtttaatctttgcttatttttactttaaattagtctttcaccattcccttgcggtactttctctatagctcctggtaagtaaatttctttctccaatactttttgagttaaatgttttagtttatgtgggggggggttagttatgttggttggttgcctcgtgcc

SEQ ID NO: 66 - DNA, 57 bp, Rattus norvegicus
tgtacaggctgtattcctcatgcccaatggcacgctgtctgccccgagtggagatct

SEQ ID NO: 67 - DNA, 186 bp, Rattus norvegicus
  misc_feature (1)..(186): Wherein n is a or t or g or c.
naatctctttgttgcctagacctgtgcccctgccacagagcctcgcagggactggtcacctgccgtgtgctggctgctgctgagtcactcttctggaagctggggcagaggtggccaagatgtcgactgagatctccactcggggcagacagcgtgccattgggcatgaggaatacagcctgtaca

SEQ ID NO: 68 - DNA, 238 bp, Rattus norvegicus
gaattcgccctaaagatgctgcaggactgtcccaaggcacgcagagaggtggagctacactggagggcctcccagtgcccacacatcgtgcacatcgtggacgtctatgagaacctgtatgccgggaggaagtgcttgctgattgtcatggagtgtctcgatggtggagagctctttagtcggatccaggaccgaggagaccaggcattcacagaaagagaggcatcagagatcatga

SEQ ID NO: 69 - DNA, 175 bp, Rattus norvegicus
ccatggtggggcctcacggctacatctctgcatctgactggcctctcatgattttttacatggtgatgtgtattgtttacatattatatggtgtcctctggctgctgtggtctgcctgttactggaaagatatactgagaatccagttctggattgcagctgttattttcctagg

SEQ ID NO: 70 - DNA, 131 bp, Rattus norvegicus
gtgcactcgaattccaggtcctacctgtggcaggaagagcccatgatgggagcttgaatctacccccattcctactgggcccagagctcccctctgaccagcagagatagcccctgccagccccagctagc

SEQ ID NO: 71 - DNA, 363 bp, Rattus norvegicus
tccggaagagcaatcagtgctcttaaccgctgagccacctctccagccctgaagggctctttcaaaggtttattctttctcctttcacaagtcggcatcgaaacttccaagtgtcctcaaagtccagggctccttggactccataacgtttctccgcaatctcaataacttccctcgcaatgttttcttgactggtgcccttcacgctgatatatttgcagtcggagctgccatagtggcaggagattgcctgcgcagaaaggaccggccggagaagggcagtttatcaatcccattgtgccccgaaaccaagcagagccctccgaagaggaatgcttcacttgggatttgatttctcaattg

SEQ ID NO: 72 - DNA, 477 bp, Rattus norvegicus
attatttatatgagtacactgtagctatcttcagacacaccagaagagggcaccagatcccattacagatggttgtgagccatcatgtggttgctgggatttgaactcaggacctccggaagagcaatcagtgctcttaaccgctgagccacctctccagccctgaagggctctttcaaaggtttattctttctcctttcacaagtcggcatcgaaacttccaagtgtcctcaaagtccagggctccttggactccataacgtttctccgcaatctcaataacttccctcgcaatgttttcttgactggtgcccttcacgctgatatatttgcagtcggagctgccatagtggcaggagattgcctgcgcagaaaggaccggccggagaagggcagtttatcaatcccattgtgccccgaaaccaagcagagccctccgaagaggaatgcttcacttgggatttgatttctcaattg

SEQ ID NO: 73 - DNA, 413 bp, Rattus norvegicus
caattgatgctgatgtgacagtgataggttctggtcctggaggatatgttgctgccatcaaagctgcccagttaggctttaagacagtctgcattgagaagaatgaaacactaggaggaacatgcttgaatgttggttgtattccttcaaaggctttattaaataattctcattattaccatttggcccatggaaaagattttgcatctaggggaattgaaataccagaagttcgcttgaatttagagaagatgatggagcagaagcgttctgcagtaaaagcattaacagggggaattgcccacttattcaaacaaaataaggttgttcatgtcaatggatttggaaagataactggcaagaatcaggttacagctacaacggccgatggcagcactcaggttattggtacc

SEQ ID NO: 74 - DNA, 154 bp, Rattus norvegicus
tgatcataatctgtgaagtgactccttgttcatgagagcagatttttaacaagacgagtatgagaggaaacctaggtaagctatgatgtataatcacataagctggtcctgtagctgtcaggtttttcagtaggaacggatagcaggaggtacc

SEQ ID NO: 75 - DNA, 726 bp, Rattus norvegicus
tttttttttttttttttcaaatactatgtggttttatttagaaataaaagattgttcgtttgtacatcatttaagaattataccagtttatcactgcacaagaatgagagacaatggtgcaactcgagtccctcacatcaagtactgccaagctgttgatcataatctgtgaagtgactccttgttcatgagagcagatttttaacaagacgagtatgagaggaaacctaggtaagctatgatgtataatcacataagctggtcctgtagctgtcaggtttttcagtaggaacggatagcaggaggtacagtagcacagtcagcctcattcaaggtcttgtcaataacaggtctgtaatccaaagtaaccttcccagtcttggtgtccacatatgagagggtgtgcttcctccagtgttccgcaaatggcttcttctgctggccctcgatgggcttggagtaatcatactcatcaatccgcaccttgtaatcttccctggcatgagctccccgtgactccttccgtgcttccgcaccatatatggtctgcagtgcgcacagcatcagattctgcagctccagcgtctccaccaggtctgtgttccagaccattcccctgtcaaacgtcttcagatgctgtaggtctccatagagctggctgactttttcacagccttcttgcagcacacttcccacacgggacacggcggcatggctctgcatcga

SEQ ID NO: 76 - DNA, 82 bp, Rattus norvegicus
tgtacggtcatttcttctgccttccgtctctgcgactctcggagaacttccagcagcagcatgttgggccagagtatccgga

SEQ ID NO: 77 - DNA, 440 bp, Rattus norvegicus
tttttttttttttttttatttatatcattagtttatttacatttttttctagtataagagttcaagagtttaatccaatttccagatcatatctcttaaactttcttcattctgttaatgggatgaattaaatatccttattttttaagtagctggtgccttactataaagaaaggagcagcaaatccagatccaaagtacacggtcatcataagcaataaccgccacttgttttccactgaaaacggcaaattcttccccggaccctcctcatagtggctgcgacgcaccacggaggtggtgaacctccggatactctggcccaacatgctgctgctggaagttctccgagagtcgcagagacggaaggcagaagaaatgaccgtaccacctcaccctactttcttcacgaccttgctatccggaacgagcctcgtgcc

SEQ ID NO: 78 - DNA, 277 bp, Rattus norvegicus
ggtaccatctcctggccatcccctcgattaaccaagctattcatgtattcttatgccagagcagtgtcaactcctggaggtcccgggtgcagcagatgcctcgtgtggtagttctaaatttaaatttcactggaaactgggcaaccaagcaatgagccacagcaaaataagagaagcatcaccaccaatgaagctgttgttaaaaccatactaccaactgcccataaaaaattactgatttgatgtattctttttcatgtcagcatatgttcaattg

SEQ ID NO: 79 - DNA, 148 bp, Rattus norvegicus
ggtaccactgttttcctagtttcctttgttatctgtccatgagtgaggtgcgtttgatcctgttgtatggcagtttcctcttgaattcccacagctgcctctagctttgtggacttggcggtggcaaccaccacggatgcagcaattg

SEQ ID NO: 80 - DNA, 347 bp, Rattus norvegicus
agatctggagaattgaaggttccaacaaggtactggtggaccccgccacatacggccagttctatggaggtgacagctacatcattctgtacaactaccgccatggtggccgccagggacagatcatctacaactggcagggtgcccagtctacccaggatgaggtcgctgcttcagccatcctgactgcccagctggatgaggaactgggaggaactcctgtccagagccgagtggtccaaggcaaagagcctgcacacctcatgagcttgtttggtgggaagcccatgatcatctacaagggtggcacctcccgagatggtgggcagacaacccctgccagtacc

SEQ ID NO: 81 - DNA, 467 bp, Rattus norvegicus
agtactggcaggggttgtctgcccaccatctcgggaggtgccacccttgtagatgatcatgggcttcccaccaaacaagctcatgaggtgtgcaggctctttgccttggaccactcggctctggacaggagttcctcccagttcctcatccagctgggcagtcaggatggctgaagcagcgacctcatcctgggtagactgggcaccctgccagttgtagatgatctgtccctggcggccaccatggcggtagttgtacagaatgatgtagctgtcacctccatagaactggccgtatgtggcggggtccaccagtaccttgttggaaccttcaattctccagatctgtttctggccagttccgtcatcatccatgccgtgctgggcagccatggcggtggaggtgtgcagtgtagcagcatcgaaaggcacgcgctccacgttggcaatgtggctggagaggtacc

SEQ ID NO: 82 - DNA, 95 bp, Rattus norvegicus
tcatgaagggcgtggagtagacactggctttgcacagagttgcccatgcctgttctcctaatccaactggaccccgtggtaggagtgcacccggc

SEQ ID NO: 83 - DNA, 535 bp, Rattus norvegicus
ttttttttttttttttttccaaggagagaggatttatttgtgttccctgggacgggaacagggagagtccagaagagccaaagtttcaaggacacaaccaggttcagagagtctagagaacccgggtgcactcctaccacggggtccagttggattaggagaacaggcatgggcaactctgtgcaaagccagtgtctactccacgcccttcatgaactccaggaactcgtcatagtcgattcggccatcgttgttcttgtcaccgtccttcatgagctcttcgatgtcatcttccgtgatggtctcacctgtggcctgcagcatcatcttcagttcatccaagtcaatgtagccatcagcgtttttgtcaaacatgcggaagagatccgacagctcctcctcagacttccctttgctgtcatccttcatgcaccgaaccatcatgacaaggaactcgtcgaagtccactgtgccactgccatcctcatctacctcgtcgatcatctcctgcagctcctcaggtgtggggttctgt

SEQ ID NO: 84 - DNA, 182 bp, Rattus norvegicus
gccggggacactgcctgggcctgagtatgggggcattctcttgatgcagtactgggcctgatccggaggcagctctcgacgaagttcctctgccaagatgtaaggcttatcagaagccagaatccggaaggaggcgatgacctgttctgcagtgtccgtgtctgcggtctctctagtcatga

SEQ ID NO: 85 - DNA, 445 bp, Rattus norvegicus
tttttttttttttttttccaggtaacaacctacacttgagcctttattgcgttctgatagggtcaggggttacagaaggagcatcagaggtcgctctccccgtagagggcagaggagaaggcagtgtagtccagggccccggggacactgcctgggcctgagtatgggggcattctcttgatgcagtactgggcctgatccggaggcagctctcgacgaagttcctctgccaagatgtaaggcttatcagaagccagaatccggaaggaggcgatgacctgttctgcagtgtccgtgtctgcggtctctctagtcatgaagtcaatgaaggactggaaggtgactgtgccttgtccgttggggtcaaccagagtcataattcggggaaactcagcttcacccaagtcatagcccatggaaatgaggcaggccctcgtgccgaatt

SEQ ID NO: 86 - DNA, 246 bp, Rattus norvegicus
tccggagtgggatgcccacttcatccatagacacactgcttaggtcctgtgcactcctcaccacccgtctgctgtcatccttggctctcctttccgcagccctgatgggcgaggtgagttctgccggggttggcactgggtcctgctcacccactcttctctctgaggcgggatctgaaagactactgagtcgtttttgctgttctcggttgtgctgcaagagcacaatggtagggttgacaattg

SEQ ID NO: 87 - DNA, 126 bp, Rattus norvegicus
caattgtattcttgctgactaaggttcaaggagactggtttttctgagaagccatccctggtaaattgacagtagttcagagagtttagtcttatcttgtcatgagctggtaaccactggggtacc

SEQ ID NO: 88 - DNA, 88 bp, Rattus norvegicus
tccggaaatgtgggagctgagcgcccggcagacacgctgctatgcaggggctatttggggcttgcttttagggatttgtttccaattg

SEQ ID NO: 89 - DNA, 370 bp, Rattus norvegicus
tgtacaggaggtgagcaaaggcaggggagaggagaggttctggagcggggttggcatgagctgggagctccacaatagccgtggccctctgagaaagaagggtagtgtttgtgaggccagatgctgctctcttggctctctgactgactggacatgctgctggccatttggctatctgcctcttcagctatggactttatttatgggaagattaaacaaggtgagaaagctcaattggaaacaaatccctaaaagcaagccccaaatagcccctgcatagcagcgtgtctgccgggcgctcagctcccacatttccggagtagcatgaaacttgtcagcccttatcctaggccctgggatgttaaagctt

SEQ ID NO: 90 - DNA, 339 bp, Rattus norvegicus
aagctcgagggtggaatcaaggtaccagaatgtggatatttcttcacccggggtgaatgtggaagctcctgatattcacgtgaaagctcccaagttcaaggtgccaggcgtggaagccgcagggccaaaaatagagggcaacttgaaaggtcccaaggtgcaggcaaacctggacacaccagacatcaatatccaaggtccggaagctaaaatcaaaaccccctcttttagtgtgtcggctcctcaagtctccatacccgatgtgaatgttaaattgaaaggaccaaacataaagggtgatgttcccagtgtgggactggagggacctgacgtagatct

SEQ ID NO: 91 - DNA, 100 bp, Rattus norvegicus
ccatgggcacaggctgcgcccgaggcttcctggcagcctttgacacggcatggatggtaaagagctgggaccagggcacccctcccctggaggtgctagc

SEQ ID NO: 92 - DNA, 44 bp, Rattus norvegicus
gctagcatgacaccaacaaggaccctatcttgaggaaaagatct

SEQ ID NO: 93 - DNA, 314 bp, Rattus norvegicus
cctaggactgtggggacacttgggccttccgcatggatcgaagggccttctcccgaaggtgcctctctaagtcatcaaggttgtcatcttcagcttcactctcagtctccttcctgggctctggtgctgccgcaggctcttcctgggctgatggagtggcggcagcagagacagctgcaggggcggcaggagctggggtggctacggccacagccttctccttcttgtgttttttgtgcttcttgtgtttcttatccttcttatgtttcttgtccttcttcttcttcttcttctttccacctccttcttgatca

SEQ ID NO: 94 - DNA, 698 bp, Rattus norvegicus
cctaggactgtggggacacttgggccttccgcatggatcgaagggccttctcccgaaggtgcctctctaagtcatcaaggttgtcatcttcagcttcactctcagtctccttcctgggctctggtgctgccgcaggctcttcctgggctgatggagtggcggcagcagagacagctgcaggggcggcaggagctggggtggctacggccacagccttctccttcttgtgttttttgtgcttcttgtgtttcttatccttcttatgtttcttgtccttcttcttcttcttcttctttccacctccttcttgatcagaattcctggcaggggacgggcttggtgttgggcttttagccttcttggctggtgctgcaggtgaccagtttgtggagggtgactgagactgcacaagagaggggggtgctggaggctttttagctgttggctcaggagatccagagacagagcgggaagatgaaacccttcttacggactgagggcttggtgaggcagccttttttatctttttgggttccggagtcctggagactctcctaataggcctagtactcggagacggggactgccttccttggggagacgctgaagctcctcttcgaacagggggagggcttgaggtctgaggcgcccgaggtcgtggtgagggcgagtgccttttgtttggttgtggtgaccgg

SEQ ID NO: 95 - DNA, 301 bp, Rattus norvegicus
ggtacttttaagataaagtctagtccagtttaaatgtcaactagtgcaaaagctagtgacaaagctggataccaaaaatagccaacactacaacataaacactttgtattcaaagtatacaattcactttataaattattaatggtatataatttgtataaaatatattgctgctgtccagcatgcttttttttaaaatccaaacacaaggccaggaggatagttaatttgaagaatagataacttccattacactacacatttaacaatgcttaaatgtttgtttactgccatgcaattg

SEQ ID NO: 96 - DNA, 660 bp, Rattus norvegicus
ttttttttttttttttttctttgaattcgtttatttaagagatagaacacagccattcaaacttgtgaaacaaagtattaacacgggataaggttggaaaattaagatgaattgctctattccatttgcacaataaatatttttaaagaagcttgtagatctttaaaagcttttaaactagatactaacataaataagcatttctatctaaattgaggcatactgattttcaatagaattataatatcaattgcatggcagtaaacaaacatttaagcattgttaaatgtgtagtgtaatggaagttatctattcttcaaattaactatcctcctggccttgtgtttggattttaaaaaaaagcatgctggacagcagcaatatattttatacaaattatataccattaataatttataaagtgaattgtatactttgaatacaaagtgtttatgttgtagtgttggctatttttggtatccagctttgtcactagcttttgcactagttgacatttaaactggactagactttatcttaaaagtacctaacccgagcctaatattttatgtcctctaaggtttcccattttgtttgggagacgtagtttgaaatttttctaacataatatccttttcaaaattgtgtctacatgaagag

SEQ ID NO: 97 - DNA, 113 bp, Rattus norvegicus
ggatccaaaataaaatcaagttcctaatggtgggaggtgtcaatcctcttgtgagaaaaagattgattgtatagcttataaaatttgcaagacaggtttaaaggagtaagctt

SEQ ID NO: 98 - DNA, 294 bp, Rattus norvegicus
gccgggggtccagaagggagagtcccagactcgctactctgcgacagggtgcgggatcgggaccgactgccatcgatggatgccgcactggtcagagatgctgtgcgagaccgagacaggcgagtcatacaggatgaggccatgtagcccatgccttgcacgaagtacttgaaagcttctgtcagcttgcctggctgagtcagctgcggctgacctccagagtccgccatcttgaggaatgaggtctgtgtggggtccagttttgaattacattccaccacggcatcttcatga

SEQ ID NO: 99 - DNA, 199 bp, Rattus norvegicus
gccggccaagggacagcaaacaatgcccctcctcccctgctcctgctgtgcagacaaggcctccatccctccatcctagcaggggttgtggaagcaggggacctgtcgggctgcagggagcatagctggctcagcatagttcacaggaagtgccatgcttacgcacttcggaagagaccccagtggatcagggtcatga

SEQ ID NO: 100 - DNA, 289 bp, Rattus norvegicus
gaattcaccaacatgatgatgaagggggggaacaaagttctggcccgatcactcatggcccagactctggaagccgtgaaaaggaagcagtttgaaaagtaccgtgcggcctcagcagaggaacaggcaaccattgaacggaacccctacaagatcttccacgaggcactgagaaactgtgagcctgtgattgggttggtgcctatcctcaaagggggtcatttctaccaggtccctgtgcctctggctgaccgacgccggcgcttcctggccatgaagtggatgatca

SEQ ID NO: 101 - DNA, 730 bp, Rattus norvegicus
ttttttttttttttttttcaagtgtttcacttttattagtggtaatatgtgtatatatgttttgtctgcacatgtgtctgtataccatgtgtataccacaacggtcagaagttgtctttggaacgggagttacaggtggttagtgagtctccacgggctgctgggaatcaaaccaggtcctttggaaagagcagtgcttttcaccactgagccatctctccagcccctcgagtggtctcttgtggcagtgtgtcctttccccacctctcctttcctgctaccaccagcggtagtgggccagggcacggttggcctcagccatcttatgcatattgtgcttcctcttgatcacgggacccctgttgtgaaaagcctccagcagctcatgcgacagcttctctggcatcagcatccgtcgaggcttgttctctcggcactctgtgatcatccacttcatggccaggaagcgccggcgtcggtcagccagaggcacagggacctggtagaaatgaccccctttgaggataggcaccaacccaatcacaggctcacagtttctcagtgcctcgtggaagatcttgtaggggttccgttcaatggttgcctgttcctctgctgaggccgcacggtacttttcaaactgcttccttttcacggcttccagagtctgggccatgagtgatcgggccagaactttgttcccccccttcatcatcatgttggtgaattc

SEQ ID NO: 102 - DNA, 294 bp, Rattus norvegicus
gaattctatcttccactgccccacccaggcccaggctgcagtagcccagtgctttgaaccggaagcagcaacactttggacaacccagcagctactttttgcagctgccacaggccatggagctgaaccgagaccacatgatccgtagcctgcagtcagtgggcctcaagctctggatctcccaggggagctacttcctcattgcagacatctcagacttcaagagcaagatgcctgacctgcctggagctgaggatgagccttatgacagacgctttgccaagtggatgatca

SEQ ID NO: 103 - DNA, 191 bp, Rattus norvegicus
cggccgcatcaccctggaagagtatcgaaatgtggtggaggaactgctctctggaaatcctcacatcgagaaggagtcagctcggtccatcgccgacggagccatgatggaggctgccagcgtgtgcgtgggacagatggaaccggaccaggtgtacgaggggatcacctttgaggacttcctgaagatct

SEQ ID NO: 104 - DNA, 593 bp, Rattus norvegicus
tgcgtaaggggtccagcggcctggccgatgagatcaacttcgaggacttcctgactatcatgtcctacttccggcccattgacactaccctgggtgaggaacaagtggagctgtctcggaaggagaagctgaaatttctgttccatatgtatgactcggacagtgacggccgcatcaccctggaagagtatcgaaatgtggtggaggaactgctctctggaaatcctcacatcgagaaggagtcagctcggtccatcgccgacggagccatgatggaggctgccagcgtgtgcgtgggacagatggaaccggaccaggtgtacgaggggatcacctttgaggacttcctgaagatctggcagggcatcgacatcgagaccaagatgcacatccgcttcctcaacatggagaccattgccctctgccactgatcgtgcaggggagggggtggctaaggaccgaggttcagccctttgtctgggctgctgtgacaatcagtaacccttcagttagcctccttgtgtggtgtggcgtgtgggactccgatgtttttatctctaatggtgacaataaaggtttcctaatgagcccgg

SEQ ID NO: 105 - DNA, 179 bp, Rattus norvegicus
  misc_feature (1)..(179): Wherein n is a or t or c or g.
ggatcccagcggatagtacacctatcactggacacatccgcgattttcaggtttcttacgggaccaggcttatccaaaacattgacagtcgcataggccacaaaactgccagctgggttagttgctgtgactacatatttaccgccatcacttcgcttcgctttggtaagggagaattn

SEQ ID NO: 106 - DNA, 160 bp, Rattus norvegicus
  misc_feature (1)..(159): Wherein n is a or t or c or g.
naattttgaacgtgacacaagctcgagtagcatctagcttgccaatggctgtgatcccatttttgacagcaaacctgtcctaccatagttttgtaagtttacctttgagtacaggtaatttgaactgtgaaatctgtacgacaacacggggtgcactagt

SEQ ID NO: 107 - DNA, 85 bp, Rattus norvegicus
tctagacaatataaactcctcataaaggcccttcagttacctgaacctgatttagaaattcaatgatttgaagcaaatatgtaca

SEQ ID NO: 108 - DNA, 700 bp, Rattus norvegicus
tttttttttttttttttaattttcaacattttatttttgtacatatttgcttcaaatcattgaatttctaaatcaggttcaggtaactgaagggcctttatgaggagtttatattgtctagacccaagatatgctgcaaaagcagtctgaagtaaagtaggaaataacatttttctaaagacaggcttagaaatagtaatccagtaattgaagatgtttcccctctgtggtagaggacttgattcatacctggcagcaaggcccccattcacgggtatagccaaaaggatggggtacagaccacccagaacaaaaccaactagtgcaccccgtgttgtcgtacagatttcacagttcaaattacctgtactcaaaggtaaacttacaaaactatggtaggacaggtttgctgtcaaaaatgggatcacagccattggcaagctagatgctactcgagcttgtgtcacgttcaaaattcgccgaaatagactgtttgctattaggcccccaaaagcagcattaagtccaatatatgctgatccatattcaagcagattcctgtctgattctggaagttgtttgatttttctgggtatgatattaaatattaaatcatctttgttagtacttggtttatgactttccatcttggaccactcggcagaatgacggcagcttatggccgcctcccgcgcccgca

SEQ ID NO: 109 - DNA, 200 bp, Rattus norvegicus
ccatggccgtgggctttgtgatgtggtccttgatgctctgcaccacccccacaagggatgaggtggccagggcagccacgctgtagttgctggggcaagctctggagtcagatatgtagccattggtggtctggaagcacctctgccaaggatcccaacagaaatccatctgcttgtccttgccagcaacatggtccgga

SEQ ID NO: 110 - DNA, 206 bp, Rattus norvegicus
tcatgaggaaagaggtcatgcaggaagtggcccagctcagccagtttgatgaagaactctataaggtgattggcaagggcagcgaaaagagcgatgacagctcctatgacgagaagtacttgattgccacctcagaacagcccatcgcagctctgcaccgggacgagtggctgcggccagaggatctgcccatcaagtacgccggc

SEQ ID NO: 111 - DNA, 572 bp, Rattus norvegicus
  misc_feature (1)..(572): Wherein n is a or t or c or g.
ccgactcctcgttgatgaagccatccagaagtntgatggggagcgggtaaagctggaagcagagcgatttgagaacctccgagagattgggaaccttctacacccctctntgcccattagtaacgatgaggatgcagacaacaaagtagagcgtatttggggtgattgtacagtcagaaagaagtattcccatgtggacctggtggtgatggtggatggctttgaaggcgaaaagggagccgtggtggctggtagtcgggggtacttcctgaagggggttcctggtgttcctggagcaggcacttatccagtatgcactgcgcaccttgggaagccgaggctacactccaatctacacncccttcttcatgaggaaagaggtcatgcaggaagtggcccagctcagccagtttgatgaagaactctataaggtgattggcaagggcagcgaaaagagcgatgacagctcctatgacgagaagtacttgattgccacctcagaacagcccatcgcagctctgcaccgggacgagtggctgcggccagaggatctgcccatcaagtacgccggc

SEQ ID NO: 112 - DNA, 184 bp, Rattus norvegicus
gctagccggctgatgaacgagagagattactggccagggtatggagaagggaacacttggtgtccaggagctcttccagaccctgagattgtaaggatggttgaagctcgacagtctctccgtgaggggtacacagaagatggtgagcaaccgcaaggcaaagggagcttcccagccatgatca

SEQ ID NO: 113 - DNA, 325 bp, Rattus norvegicus
actagtgtctaccgcacacctttaaatctaaccttgaagaattctgtggcagccatgggtgggaccgacggcaaagaagacggcgaacagtttaaatccgttctccattgggacatgaagtccaaggccggagcgggggcggctagccggctgatgaacgagagagattactggccagggtatggagaagggaacacttggtgtccaggagctcttccagaccctgagattgtaaggatggttgaagctcgacagtctctccgtgaggggtacacagaagatggtgagcaaccgcaaggcaaagggagcttcccagccatgatca

SEQ ID NO: 114 - DNA, 337 bp, Rattus norvegicus
cctaggaaacattggagccttaaggcgggctacagacaagaacagtttagccatgcgggtcattcttcactgtttggcaaccttacttttttccctctctgccttcctgtgtcttgcattccatttgtgggactgtatttgaaaggccaggcatgtaaattccattagagcaaggtctctcctggaatggaacgaatcattgactcaatctttctcttttcccaggaagtgtcaaaataactctccgagcagctgcagcttaggaggaacggttgtgagaccgtccagcagctatcttccaccactcagggttgtcgctcacaccccttaaggatcc

SEQ ID NO: 115 - DNA, 62 bp, Rattus norvegicus
gaattcacacagattgatcctatcctgtctgtgaaaagcaagaagtgcagatgtgttcatga

SEQ ID NO: 116 - DNA, 535 bp, Rattus norvegicus
tccatttgttgtcgtttttttttttttgggtaaacaaagggttaatttattatataaggtaagccaataagctctcatgtactacagagagaaaacatacagtgcgcataataaatgaattccatatactgagaataaataggataagcgtagtaaagaggaagtcaagaggagcccacagttatagccacatgacgagaagttaaaagaaaaataaaagaagaagtccggggagaaaccactttatttatttggagccatgcacttgttttagtgccaagggcacaggaagatggacaagataaggtcctgatcacaccagatgcttagaaagatctttcagtgtctaacctacatctagaagagtcatgaggagtagtggcagggtgtgtgcgccacaacctttgaggaaagcgatccttatacacagggcgaccccaacaaccctgtcattttatcatgaacacatctgcacttcttgcttttcacagacaggataggatcaatctgtgtgaattcgatttgggtatatcga

SEQ ID NO: 117 - DNA, 378 bp, Rattus norvegicus
aaattgcgattagggtagctatacatggagggcaagcagggcagtgcttggtaggtggtgcggcctcgcgcgtatgtctgtagaaacagttgcttataggggccaaactggactactcccacctggtcatgaagaagccgcatagctgtttcaaaagaacctgccaggatgtgatccactggaagctgggagttattgcaccagatttgagttgggcttgttcccttggttgggggcacaaagaaaccgtcttcagcaccaccggcaaccccagagggtacatccagctcaggtgggagctccaaatcttcttctacgtcccagccacctccttcctcttgtcccttgccgagagtatcctcccccaaaccttccgga